(19) United States
(12) Patent Application Publication
(10) Pub. No.: US 2019/0031785 A1
(43) Pub. Date: Jan. 31, 2019

(54) MULTISPECIFIC IMMUNOMODULATORY ANTIGEN-BINDING CONSTRUCTS

(71) Applicant: Compass Therapeutics LLC, Cambridge, MA (US)

(72) Inventors: Thomas Joseph SCHUETZ, Cambridge, MA (US); Piotr BOBROWICZ, Cambridge, MA (US); Helen SABZEVARI, Cambridge, MA (US); Michael March SCHMIDT, Cambridge, MA (US); Robert V. TIGHE, III, Cambridge, MA (US); Simon METENOU, Cambridge, MA (US)

(21) Appl. No.: 16/069,981

(22) PCT Filed: Jan. 13, 2017
(86) PCT No.: PCT/US17/13512

§ 371 Date: Jul. 13, 2018

Related U.S. Application Data
(60) Provisional application No. 62/278,359, filed on Jan. 13, 2016.
(60) Provisional application No. 62/361,842, filed on Jul. 13, 2016.

(51) Int. Cl. C07K 16/46; C07K 16/32; C07K 16/28; A61K 47/68; A61P 35/00

(52) U.S. Cl. CPC C07K 16/468; C07K 16/32; C07K 16/2809; C07K 16/2818; A61K 47/6879; C07K 2317/24; A61P 35/00; C07K 2317/55; C07K 2317/622; C07K 2317/31; C07K 2317/21; C07K 16/2878

(57) ABSTRACT

Provided herein are multispecific immunomodulatory antigen-binding constructs (MIACs) and compositions comprising the constructs. Also provided are methods of using the constructs and methods of making the constructs.

### CROSS REFERENCE TO RELATED APPLICATIONS

This application claims the benefit of U.S. Provisional Application No. 62/278,359, filed Jan. 13, 2016, and U.S. Provisional Application No. 62/361,842, filed Jul. 13, 2016, each of which is herein incorporated by reference, in its entirety, for all purposes.

### SEQUENCE LISTING

The instant application contains a Sequence Listing which has been submitted electronically in ASCII format and is hereby incorporated by reference in its entirety. Said ASCII copy, created on Jan. 11, 2017, is named 35868PCT_CRF_sequencelisting.txt and is 94,734 bytes in size.

### FIELD

Provided herein are multispecific immunomodulatory antigen-binding constructs (MIACs) and compositions comprising the MIACs. Also provided are methods of using the constructs and methods of making the constructs.

### BACKGROUND

Simultaneous Modulation of Activating and Inhibitory Receptors on the Same Effector Cell

Effector cell-based immunomodulatory antibody therapies utilize antibodies to recruit effector cells, such as T cells and natural killer (NK) cells, to a desired site of action, such as a tumor.

One example of an effector cell-based immunomodulatory antibody therapy is the Bispecific T Cell Engager (BiTE®) Immunotherapy platform from Amgen Inc. The BiTE® platform is designed to function as a bridge between T cells and cancer cells. BiTE® molecules have two antigen-binding sites: (1) a binding site that binds an antigen on the surface of a tumor cell; and (2) a CD3 binding site, which binds to CD3 on the surface of T cells. By binding both antigens, BiTE® molecules leverage the cytotoxic activity of T cells to destroy cancer cells expressing particular antigens. See Nagorsen and Baeuerle, Exp. Cell. Res., 2011, 317:1255-1260; and Baeuerle et al., Curr. Opin. Mol. Ther., 2009, 11:22-30, each of which is incorporated by reference in its entirety.

Other effector cell based immunomodulatory antibody therapies include molecules that engage an antigen expressed on a cancer cell, and the costimulatory (i.e., activating) receptors CD3 and CD28 expressed on the surface of a T cell. See Wang et al., J. Biochem., 2004, 135:555-565, incorporated by reference in its entirety.

However, evidence is accumulating that the provision of only canonical signals 1 and 2 (i.e., via CD3 and CD28, respectively) cannot be sufficient to induce therapeutically relevant activation of effector cells in the context of cancer therapy. One reason for this is that some cancer cells express ligands for inhibitory receptors expressed by effector cells. For example, PD-L1 expression by a cancer cell activates the inhibitory receptor PD-1. Inhibitory receptors transduce an inhibitory signal that abrogates stimulatory signals from co-engaged activating receptors on the same effector cell.

Additionally, some cancer cells fail to display ligands for activating receptors expressed by effector cells. For example, cancer cell shedding of MICA or MICB means that these ligands will fail to stimulate the activating receptor NKG2D.

Without being bound by theory, it appears that the signals transmitted by these opposing types of receptors (i.e., activating and inhibiting) are integrated at the level of the individual cell, and that each cell then becomes activated (or not) according to the integration (e.g., summation) of the activating and inhibitory signals transduced at its immunological synapse. For example, the activation of NK cells is dependent on, among other things, the integration of signaling through both activating receptors such as NKG2D, and inhibitory receptors such as inhibitory forms of KIR and PD-1. See Pegram et al., Immunology and Cell Biology, 2011, 89:216-224, incorporated by reference in its entirety.

Current effector cell based immunomodulatory antibody therapies do not account for these integrative signaling mechanisms. The current therapies therefore lack the ability to activate effector cells in certain therapeutic contexts, such as malignancies that express ligands for the inhibitory receptors expressed by effector cells.

There is therefore a need for novel, cancer cell-targeted, therapies that optimize effector cell function by simultaneously modulating signaling through both activating and inhibitory receptors expressed by the same effector cell. Thus, in some embodiments, provided herein are molecules that perform this function. Also provided are methods of using such molecules, for example, in therapeutic applications utilizing effector cells. Also provided are methods of making these molecules.

Specific Targeting of Immunomodulatory Constructs to Cancer Cells

Certain cancer cells either (i) fail to express a ligand for an activating receptor expressed by an effector cell; or (ii) express a ligand for an inhibitory receptor expressed by an effector cell. The resulting failure of the cancer cell to stimulate an activating signal in the effector cell, or the success of the cancer cell in stimulating an inhibitory signal in the effector cell, respectively, can also promote the progression of cancer.

For example, Hodi et al. (New Engl. J. Med, 2010, 363: 711-723), incorporated by reference in its entirety, describes improved survival in patients with metastatic melanoma receiving the anti-CTLA-4 antibody ipilimumab, which antagonizes an inhibitory receptor. While administration of ipilimumab did increase survival, the antibody is not specifically targeted to cancer cells, and therefore broadly, and non-specifically, activates the immune system. The consequences of such broad activation include severe autoimmune toxicity such as colitis and endocrinopathies. See id.

Thus, current effector cell-based immunomodulatory therapies are often potent but nonspecific. On the contrary, traditional monoclonal antibody-based therapies targeting cell surface antigens on cancer cells are generally very specific, but can lack potency.

Accordingly, there is also a need for novel therapeutics that address the lack of specificity inherent in many effector cell-based immunomodulatory antibody therapies, and the lack of potency of many cancer cell-targeted monoclonal antibody therapies. A useful molecule would combine the specificity of a cancer cell-targeted monoclonal antibody with the immunomodulatory capability of an effector cell-based immunomodulatory therapy. Thus, in some embodiments, provided herein are molecules that perform this function. Also provided are methods of using such molecules, for example, in therapeutic applications utilizing effector cells. Also provided are methods of making these molecules.

This application is related to co-owned patent application PCT/US2016/013291, filed Jan. 13, 2016, which is herein incorporated by reference, in its entirety, for all purposes.

### SUMMARY

Described herein is a multispecific immunomodulatory antigen-binding construct (MIAC) polypeptide, comprising: an antigen-binding module 1 (ABM1) that binds specifically to an antigen expressed by a cancer cell; an antigen-binding module 2 (ABM2) that binds specifically to an activating receptor expressed by an effector immune cell, wherein binding of ABM2 to the activating receptor agonizes the activating receptor; and an antigen-binding module 3 (ABM3) that binds specifically to an inhibitory receptor expressed by the effector immune cell, wherein the binding of ABM3 to the inhibitory receptor antagonizes the inhibitory receptor, wherein ABM1, ABM2, and ABM3 are operably linked to each other, and optionally wherein each antigen binding module is capable of binding its respective antigen or receptor at the same time as each of the other antigen binding modules is bound to its respective antigen or receptor.

In some aspects, the MIAC further comprises Fc, wherein ABM1 is an scFv fragment, ABM2 is a Fab fragment, and ABM3 is an scFv fragment, wherein ABM2 is linked to Fc, ABM3 is linked to ABM2, and ABM1 is linked to Fc, wherein the MIAC induces a greater amount of at least one of IFN-γ, TNF-α, IL-2, and granzyme B secretion by an effector immune cell upon binding to at least one effector immune cell and at least one cancer cell relative to a control set of antibodies, wherein the control set of antibodies consists of separate monospecific antibodies present at equimolar concentrations that collectively bind specifically to the same targets as the MIAC, wherein the MIAC induces a greater level of effector immune cell proliferation upon binding to at least one effector immune cell and at least one cancer cell relative to the control set of antibodies, and wherein the MIAC induces a greater level of effector immune cell CD25 cell surface expression upon binding to at least one effector immune cell and at least one cancer cell relative to the control set of antibodies.

In some aspects, the MIAC consists of ABM1, ABM2, ABM3, and Fc linked together, wherein ABM1 is an scFv fragment, ABM2 is a Fab fragment, and ABM3 is an scFv fragment, wherein the C terminus of the heavy chain of ABM2 is linked to the N terminus of Fc, ABM1 is linked to the C terminus of Fc, and ABM3 is linked to the C terminus of the light chain of ABM2, wherein the MIAC induces a greater amount of at least one of IFN-γ, TNF-α, IL-2, and granzyme B secretion by an effector immune cell upon binding to at least one effector immune cell and at least one cancer cell relative to a control set of antibodies, wherein the control set of antibodies consists of separate monospecific antibodies present at equimolar concentrations that collectively bind specifically to the same targets as the MIAC, wherein the MIAC induces a greater level of effector immune cell proliferation upon binding to at least one effector immune cell and at least one cancer cell relative to the control set of antibodies, and wherein the MIAC induces a greater level of effector immune cell CD25 cell surface expression upon binding to at least one effector immune cell and at least one cancer cell relative to the control set of antibodies.

In some aspects, the MIAC consists of ABM1, ABM2, ABM3, and Fc linked together, wherein ABM1 is an scFv fragment, ABM2 is a Fab fragment, and ABM3 is an scFv fragment, wherein the C terminus of the heavy chain of ABM2 is linked to the N terminus of Fc, ABM1 is linked to the C terminus of Fc, and ABM3 is linked to the C terminus of the light chain of ABM2.

A MIAC can comprise ABM1, ABM2, and ABM3 and a scaffold that is Fc.

ABM1 and ABM2 can be linked to a position distinct from the C terminus of Fc; and ABM3 can be linked to the C terminus of Fc. ABM1 and ABM3 can be linked to a position distinct from the C terminus of Fc; and ABM2 can be linked to the C terminus of Fc. In certain aspects, ABM3 is linked to the C terminus of Fc. In certain aspects, ABM2 is linked to the C terminus of Fc. In certain aspects, ABM1 is linked to the N terminus of Fc. In certain aspects, ABM1 is a Fab fragment linked to the N terminus of Fc. ABMs and Fc can be linked in a format that does not substantially interfere with ADCC directed against the cancer cell.

ABM3 and ABM2 can be linked to a position distinct from the C terminus of Fc; and ABM1 can be linked to the C terminus of Fc. In certain aspects, ABM3 is linked to the N terminus of Fc. In certain aspects, ABM2 is linked to the N terminus of Fc. In certain aspects, ABM1 is linked to the C terminus of Fc. ABMs and Fc can be linked in a format that substantially interferes with ADCC directed against the cancer cell.

A MIAC can comprise ABM1, ABM2, and ABM3, wherein ABM1 is anti-EGFR, ABM2 is anti-CD137, and ABM3 is anti-PD-1.

Also described herein is a multispecific immunomodulatory antigen-binding construct (MIAC) comprising: an antigen-binding module 1 (ABM1) that binds specifically to an antigen expressed by a cancer cell; and an antigen-binding module 2 (ABM2) that binds specifically to an activating receptor expressed by an effector immune cell, wherein binding of ABM2 to the activating receptor agonizes the activating receptor, wherein ABM1 and ABM2 are operably linked to each other, and optionally wherein each antigen binding module is capable of binding its respective antigen or receptor at the same time as each of the other antigen binding modules is bound to its respective antigen or receptor. In some aspects, the MIAC further comprises Fc, optionally wherein ABM2 is a Fab fragment and ABM1 is an scFv fragment, optionally wherein the C terminus of the heavy chain of ABM2 is linked to the N terminus of Fc and ABM1 is linked to the C terminus of Fc, optionally wherein the MIAC is a dimer, optionally wherein the dimer is a homodimer.

A MIAC can comprise ABM1 and ABM2, wherein ABM1 is anti-CD19 and ABM2 is anti-CD137.

A MIAC can comprise ABM1 and ABM2, wherein ABM1 is anti-EGFR and ABM2 is anti-CD137.

ABM1 can be linked to the N terminus of Fc and ABM2 can be linked to the C terminus of Fc. In some aspects, ABM1 is a Fab fragment linked to the N terminus of Fc; and ABM2 is linked to the C terminus of Fc. In some aspects, ABM2 is linked to the C terminus of Fc. In some aspects, ABM1 is linked to the N terminus of Fc. ABMs and Fc can be linked in a format that does not substantially interfere with ADCC directed against the cancer cell.

ABM2 can be linked to the N terminus of Fc and ABM1 can be linked to the C terminus of Fc. In some aspects, ABM2 is linked to the N terminus of Fc; and ABM1 is an scFv fragment and is linked to the C terminus of Fc. In some aspects, ABM2 is linked to the N terminus of Fc. In some aspects, ABM1 is linked to the C terminus of Fc. ABMs and Fc can be linked in a format that substantially interferes with ADCC directed against the cancer cell.

Also described herein is a multispecific immunomodulatory antigen-binding construct (MIAC) comprising: an antigen-binding module 1 (ABM1) that binds specifically to an antigen expressed by a cancer cell; and an antigen-binding module 3 (ABM3) that binds specifically to an inhibitory receptor expressed by the effector immune cell, wherein the binding of ABM3 to the inhibitory receptor antagonizes the inhibitory receptor, wherein ABM1 and ABM3 are operably linked to each other, and optionally wherein each antigen binding module is capable of binding its respective antigen or receptor at the same time as each of the other antigen binding modules is bound to its respective antigen or receptor. In some aspects, the MIAC further comprises Fc, optionally wherein ABM3 is a Fab fragment and ABM1 is an scFv fragment, optionally wherein the C terminus of the heavy chain of ABM3 is linked to the N terminus of Fc and ABM1 is linked to the C terminus of Fc, optionally wherein the MIAC is a dimer, optionally wherein the dimer is a homodimer.

A MIAC can comprise ABM1 and ABM3, wherein ABM1 is anti-CD20 and ABM3 is anti-PD-1.

A MIAC can comprise ABM1 and ABM3, wherein ABM1 is anti-CD19 and ABM3 is anti-PD-1.

A MIAC can comprise ABM1 and ABM3, wherein ABM1 is anti-EGFR and ABM3 is anti-PD-1.

ABM1 can be linked to the N terminus of Fc and ABM3 can be linked to the C terminus of Fc. In some aspects, ABM1 is a Fab fragment linked to the N terminus of Fc; and ABM3 is linked to the C terminus of Fc. In some aspects, ABM3 is linked to the C terminus of Fc. In some aspects, ABM1 is linked to the N terminus of Fc. ABMs and Fc can be linked in a format that does not substantially interfere with ADCC directed against the cancer cell.

ABM3 can be linked to the N terminus of Fc and ABM1 can be linked to the C terminus of Fc. In some aspects, ABM3 is linked to the N terminus of Fc; and ABM1 is an scFv fragment and is linked to the C terminus of Fc. In some aspects, ABM3 is linked to the N terminus of Fc. In some aspects, ABM1 is linked to the C terminus of Fc. ABMs and Fc can be linked in a format that substantially interferes with ADCC directed against the cancer cell.

In some aspects, a MIAC further comprises a scaffold, optionally wherein the scaffold is Fc, optionally wherein the Fc is human Fc, optionally wherein the Fc is human IgG Fc, optionally wherein each ABM is linked to the scaffold directly or indirectly with or without a linker, optionally wherein the linker is a polypeptide linker. In some aspects, Fc is an IgG (IgG1, IgG2, IgG3, IgG4), an IgA (IgA1, IgA2), an IgD, an IgE, or an IgM, optionally wherein Fc is modified, optionally wherein the modification reduces glycosylation, and optionally wherein the modification reduces ADCC.

In some aspects, each ABM is an antibody or an antigen-binding fragment thereof. In some aspects, an antibody or antigen-binding fragment thereof is an IgG (IgG1, IgG2, IgG3, IgG4), an IgA (IgA1, IgA2), an IgD, an IgE, an IgM, a DVD-Ig, and/or a heavy chain antibody. In some aspects, an antibody or antigen-binding fragment thereof is an Fv fragment, a Fab fragment, a F(ab′)₂ fragment, a Fab′ fragment, an scFv fragment, an scFv-Fc fragment, and/or a single-domain antibody or antigen binding fragment thereof. In some aspects, an antibody or antigen-binding fragment thereof is monoclonal, human, humanized, and/or chimeric.

In some aspects, at least one ABM further comprises an alternative scaffold, or another portion of the MIAC further comprises an alternative scaffold.

In some aspects, the antigen expressed by the cancer cell is a tumor-associated antigen or a tumor-specific antigen. In some aspects, the antigen expressed by the cancer cell is selected from HER2, CD20, 9-O-acetyl-GD3, βhCG, A33 antigen, CA19-9 marker, CA-125 marker, calreticulin, carboanhydrase IX (MN/CA IX), CCR5, CCR8, CD19, CD22, CD25, CD27, CD30, CD33, CD38, CD44v6, CD63, CD70, CC123, CD138, carcinoma embryonic antigen (CEA; CD66e), desmoglein 4, E-cadherin neoepitope, endosialin, ephrin A2 (EphA2), epidermal growth factor receptor (EGFR), epithelial cell adhesion molecule (EpCAM), ErbB2, fetal acetylcholine receptor, fibroblast activation antigen (FAP), fucosyl GM1, GD2, GD3, GM2, ganglioside GD3, Globo H, glycoprotein 100, HER2/neu, HER3, HER4, insulin-like growth factor receptor 1, Lewis-Y, LG, Ly-6, melanoma-specific chondroitin-sulfate proteoglycan (MCSCP), mesothelin, MUC1, MUC2, MUC3, MUC4, MUC5_{AC}, MUC5_{B}, MUC7, MUC16, Müllerian inhibitory substance (MIS) receptor type II, plasma cell antigen, poly SA, PSCA, PSMA, sonic hedgehog (SHH), SAS, STEAP, sTn antigen, TNF-alpha precursor, and combinations thereof. In some aspects, the antigen expressed by the cancer cell is EGFR. In some aspects, the antigen expressed by the cancer cell is CD19. In some aspects, the antigen expressed by the cancer cell is CD20.

In some aspects, the activating receptor is selected from 2B4 (CD244), α₄β₁ integrin, β₂ integrins, CD2, CD16, CD27, CD38, CD96, CD100, CD160, CD137, CEACAM1 (CD66), CRTAM, CS1 (CD319), DNAM-1 (CD226), GITR (TNFRSF18), activating forms of KIR, NKG2C, NKG2D, NKG2E, one or more natural cytotoxicity receptors, NTB-A, PEN-5, and combinations thereof, optionally wherein the β₂ integrins comprise CD11a-CD18, CD11b-CD18, or CD11c-CD18, optionally wherein the activating forms of KIR comprise KIR2DS1, KIR2DS4, or KIR-S, and optionally wherein the natural cytotoxicity receptors comprise NKp30, NKp44, NKp46, or NKp80. In some aspects, the activating receptor is CD137.

In some aspects, the inhibitory receptor is selected from KIR, ILT2/LIR-1/CD85j, inhibitory forms of KIR, KLRG1, LAIR-1, NKG2A, NKR-P1A, Siglec-3, Siglec-7, Siglec-9, and combinations thereof, optionally wherein the inhibitory forms of KIR comprise KIR2DL1, KIR2DL2, KIR2DL3, KIR3DL1, KIR3DL2, or KIR-L.

In some aspects, the activating receptor is selected from CD3, CD2 (LFA2, OX34), CD5, CD27 (TNFRSF7), CD28, CD30 (TNFRSF8), CD40L, CD84 (SLAMF5), CD137 (4-1BB), CD226, CD229 (Ly9, SLAMF3), CD244 (2B4, SLAMF4), CD319 (CRACC, BLAME), CD352 (Ly108, NTBA, SLAMF6), CRTAM (CD355), DR3 (TNFRSF25), GITR (CD357), HVEM (CD270), ICOS, LIGHT, LTβR (TNFRSF3), OX40 (CD134), NKG2D, SLAM (CD150, SLAMF1), TCRα, TCRβ, TCRδγ, TIM1 (HAVCR, KIM1), and combinations thereof.

In some aspects, the inhibitory receptor is selected from PD-1 (CD279), 2B4 (CD244, SLAMF4), B71 (CD80), B7H1 (CD274, PD-L1), BTLA (CD272), CD160 (BY55, NK28), CD352 (Ly108, NTBA, SLAMF6), CD358 (DR6), CTLA-4 (CD152), LAG3, LAIR1, PD-1H (VISTA), TIGIT (VSIG9, VSTM3), TIM2 (TIMD2), TIM3 (HAVCR2, KIM3), and combinations thereof. In some aspects, the inhibitory receptor is PD-1.

In some aspects, the effector immune cell is a T cell or a natural killer (NK) cell, optionally wherein the T cell is a CD4+ helper T cell or a CD8+ cytotoxic T cell.

In some aspects, the cancer cell is a cell from acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In some aspects, ABM2 comprises four immunoglobulin variable domains when present. In some aspects, ABM1 comprises two immunoglobulin variable domains when present. In some aspects, ABM3 comprises two immunoglobulin variable domains when present. In some aspects, ABM2 is a Fab fragment when present, ABM1 is an scFv fragment when present, and ABM3 is an scFv fragment when present.

In some aspects, a MIAC further comprises Fc, and ABM2 is linked to Fc when present, ABM3 is linked to ABM2 when present, and ABM1 is linked to Fc when present. In some aspects, the C terminus of the heavy chain of ABM2 is linked to the N terminus of Fc when present, ABM1 is linked to the C terminus of Fc when present, and ABM3 is linked to the C terminus of the light chain of ABM2 when present.

In some aspects, each linkage is direct or via a linker, optionally wherein the linker is a polypeptide linker, optionally wherein the polypeptide linker is a gly-ser linker or an immunoglobulin hinge region or portion thereof.

In some aspects, a MIAC is a dimer, optionally wherein the dimer is a homodimer.

In some aspects, a MIAC further comprises an antigen-binding module 4 (ABM4) that binds specifically to a further molecule expressed by the effector immune cell. In some aspects, the further molecule expressed by the effector immune cell is selected from CD16 (CD16a, CD16b), CD32a, CD64, and CD89. In some aspects, ABM4 is an Fc.

In some aspects, at least two ABMs are covalently associated with each other. In some aspects, the covalent association is in the form of a fusion protein. In some aspects, at least two ABMs are non-covalently associated with each other.

In some aspects, a MIAC induces a greater amount of at least one of IFN-γ, TNF-α, IL-2, and granzyme B secretion by an effector immune cell upon binding to at least one effector immune cell and at least one cancer cell relative to a control set of antibodies, wherein the control set of antibodies consists of separate monospecific antibodies present at equimolar concentrations that collectively bind specifically to the same targets as the MIAC. In some aspects, the amount of IFN-γ, TNF-α, IL-2, and/or granzyme B secretion induced by the MIAC is about 2, 3, 4, 5, 6, 7, or 8-fold greater than that induced by the control set of antibodies.

In some aspects, a MIAC induces a greater level of effector immune cell proliferation upon binding to at least one effector immune cell and at least one cancer cell relative to a control set of antibodies, wherein the control set of antibodies consists of separate monospecific antibodies present at equimolar concentrations that collectively bind specifically to the same targets as the MIAC. In some aspects, the level of proliferation induced by the MIAC is about 2, 3, 4, 5, 6, 7, or 8-fold greater than that induced by the control set of antibodies.

In some aspects, a MIAC induces a greater level of effector immune cell CD25 cell surface expression upon binding to at least one effector immune cell and at least one cancer cell relative to a control set of antibodies, wherein the control set of antibodies consists of separate monospecific antibodies present at equimolar concentrations that collectively bind specifically to the same targets as the MIAC. In some aspects, the CD25 expression induced by the MIAC is about 2, 3, 4, 5, 6, 7, or 8-fold greater than that induced by the control set of antibodies.

In some aspects, a MIAC induces a greater level of cancer cell death upon binding to at least one effector immune cell and at least one cancer cell relative to a control set of antibodies, wherein the control set of antibodies consists of separate monospecific antibodies present at equimolar concentrations that collectively bind specifically to the same targets as the MIAC.

In some aspects, each of ABM binds its respective antigen or receptor at the same time as each of the other antigen binding modules is bound to its respective antigen or receptor, and optionally wherein the affinity of each binding module to its respective antigen or receptor is about 0.3 nM to about 1.7 nM, 0.37 to 1.66 nM, 0.3, 0.4, 0.5, 0.6, 0.7, 0.8, 0.9, 1.0, 1.1, 1.2, 1.3. 1.4, 1.5, 1.6, or 1.7 nM when each of ABM is simultaneously bound to its respective antigen or receptor.

Also disclosed herein is a conjugate comprising a MIAC and an agent. In some aspects, the agent is selected from a therapeutic agent, a diagnostic agent, a masking moiety, a cleavable moiety, and combinations thereof. In some aspects, the agent is attached to the MIAC with a linker.

Also disclosed herein is a pharmaceutical composition comprising a MIAC or a conjugate disclosed herein and an excipient.

Also disclosed herein is a method of treating a subject with cancer comprising administering an effective amount of a MIAC or a conjugate or a pharmaceutical composition disclosed herein to the subject.

Also disclosed herein is a method of inhibiting or reducing cancer growth comprising contacting the cancer with an effective amount of a MIAC or a conjugate or a pharmaceutical composition disclosed herein to the subject.

In some aspects, a MIAC binds a cancer cell and an effector cell. In some aspects, a MIAC binds two or more effector cells. In some aspects, a MIAC agonizes an activating receptor on an effector cell and antagonizes an inhibitory receptor on the effector cell. In some aspects, a MIAC activates an effector cell. In some aspects, the activated effector cell exhibits a phenotype selected from cytotoxicity toward cancer cells, proliferation, secretion of IL-2, secretion of interferon gamma, upregulation of LAMP-1, downregulation of CD16, upregulation of CD69, and upregulation of KLRG1. In some aspects, the proliferation induced by the MIAC is greater than proliferation induced by a MIAC without ABM3.

In some aspects, a cancer is selected from acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In some aspects, a method disclosed herein further comprises administering at least one further agent to the subject.

Also disclosed herein is a composition comprising at least one polynucleotide or a set of polynucleotides encoding a MIAC disclosed herein.

Also disclosed herein is a cell comprising at least one polynucleotide or a set of polynucleotides encoding a MIAC disclosed herein.

Also disclosed herein is a method of making a MIAC, comprising expressing a MIAC in a cell comprising at least one polynucleotide or a set of polynucleotides encoding a MIAC disclosed herein.

Also disclosed herein is a method of making a MIAC, comprising expressing the ABMs of a MIAC disclosed herein, and assembling the ABMs to form a MIAC.

Also disclosed herein is a vector or set of vectors comprising at least one polynucleotide or a set of polynucleotides encoding a MIAC disclosed herein.

Also disclosed herein is a kit comprising a MIAC disclosed herein and instructions for use, optionally wherein the instructions for use include instructions for practicing a method disclosed herein.

### BRIEF DESCRIPTION OF THE DRAWINGS

FIG. 1 provides a schematic of a multispecific immunomodulatory antigen-binding construct of this disclosure (i.e., a MIAC; 101). The MIAC comprise three antigen-binding modules (ABMs; 102, 103, and 104). Antigen-binding module 1 (ABM1; 102) comprises a cancer cell-specific antigen-binding site. Antigen-binding module 2 (ABM2; 103) comprises a binding site with agonist activity toward an activating receptor expressed by an effector cell. Antigen-binding module 3 (ABM3; 104) comprises a binding site with antagonist activity toward an inhibitory receptor expressed by the effector cell. This schematic is for purposes of illustrating the components of a MIAC provided herein, and is not limiting with respect to the arrangement or number of the individual ABMs. As described in more detail below, the ABMs can be arranged differently, depending on the nature of the molecules that form the ABMs (e.g., immunoglobulins, antibody fragments, and alternative scaffolds). The ABMs can also be present in different quantities, to vary the valency of the interactions and/or provide improved control over effector cell activation.

FIGS. 2A-2D provide schematics of certain illustrative embodiments of MIACs provided herein. These illustrative embodiments are assembled from an IgG and two scFvs. FIG. 2A provides a schematic of an embodiment where two scFvs are attached to the C-termini of the IgG heavy chains. FIG. 2B provides a schematic of an embodiment where two scFvs are attached to the C-termini of the IgG light chains. FIGS. 2C and 2D provide schematics of embodiments where one scFv is attached to the C-terminus of an IgG light chain and one scFv is attached to the C-terminus of an IgG heavy chain. In FIGS. 2A-2D, ABM1 is designated as 201, ABM2 is designated as 202, and ABM3 is designated as 203.

FIGS. 3A-3D provides schematics of further illustrative embodiments of MIACs provided herein. The illustrative embodiments provided in FIGS. 3A-3D show MIACs in which the activation of an effector cell can be modulated by varying the number of binding sites for the activating and inhibitory receptors, as well as their locations within the MIAC. These illustrative embodiments are assembled from an IgG and three scFvs. FIG. 3A provides a schematic of an embodiment where one ABM2 (202) is attached to the C-terminus of one IgG heavy chain, one ABM3 (203) is attached to the C-terminus of the other IgG heavy chain, and one ABM3 (203) is attached to the C-terminus of one IgG light chain FIG. 3B provides a schematic of an embodiment where one ABM2 (202) is attached to the C-terminus of one IgG light chain, one ABM3 (203) is attached to the C-terminus of the other IgG light chain, and one ABM3 (203) is attached to the C-terminus of one IgG heavy chain. FIG. 3C provides a schematic of an embodiment where one ABM2 (202) is attached to the C-terminus of one IgG light chain, one ABM2 (202) is attached to the C-terminus one IgG heavy chain, and one ABM3 (203) is attached to the C-terminus of the other IgG heavy chain. FIG. 3D provides a schematic of an embodiment where one ABM2 (202) is attached to the C-terminus of one IgG light chain, one ABM2 (202) is attached to the C-terminus one IgG heavy chain, and one ABM3 (203) is attached to the C-terminus of the other IgG light chain.

FIGS. 4A-4B provides schematics of further illustrative embodiments of MIACs provided herein. These illustrative embodiments are assembled from an IgG and four scFvs. FIG. 4A provides a schematic of an embodiment where ABM2 (202) is attached to the C-termini of the heavy and light chains of one half of the IgG molecule, and ABM3 (203) is attached to the C-termini of the heavy and light chains of the other half of the IgG molecule. FIG. 4B provides a schematic of an embodiment where ABM2 (202) is attached to the C-terminus of one IgG light chain and the C-terminus of one IgG heavy chain, while ABM3 (203) is attached to the C-terminus of the other IgG light chain and the C-terminus of the other IgG heavy chain. In FIGS. 4A-4B, ABM1 is designated as 201, ABM2 is designated as 202, and ABM3 is designated as 203.

FIGS. 5A-5B provide schematics of further illustrative embodiments of MIACs provided herein, wherein either ABM2 (FIG. 5A) or ABM3 (FIG. 5B) are an IgG, and the remaining ABMs are scFvs attached to the C-termini of the IgG heavy chains. In FIGS. 5A and 5B, ABM1 is designated as 201, ABM2 is designated as 202, and ABM3 is designated as 203.

FIG. 6 provides a schematic of a further illustrative embodiment a MIAC provided herein, wherein each of ABM1, ABM2, and ABM3 are an scFv. In FIG. 6, ABM1 is designated as 201, ABM2 is designated as 202, and ABM3 is designated as 203.

FIGS. 7A-7C provide schematics of further illustrative embodiments of MIACs provided herein, wherein one ABM is bound to both of the other two ABMs, but the other two ABMs are not bound to each other. In FIGS. 7A-7C, ABM1 is designated as 201, ABM2 is designated as 202, and ABM3 is designated as 203.

FIG. 8 provides a schematic of a further illustrative embodiment of a MIAC provided herein, wherein each ABM is bound to two other ABMs. In FIG. 8, ABM1 is designated as 201, ABM2 is designated as 202, and ABM3 is designated as 203.

FIG. 9 provides a schematic of a multispecific immunomodulatory antigen-binding construct of this disclosure (i.e., a MIAC; 101). The MIAC comprise two antigen-binding modules (ABMs; 102 and 103). Antigen-binding module 1 (ABM1; 102) comprises a cancer cell-specific antigen-binding site. Antigen-binding module 2 (ABM2; 103) comprises a binding site with agonist activity toward an activating receptor expressed by an effector cell. This schematic is for purposes of illustrating the components of a MIAC provided herein, and is not limiting with respect to the arrangement or number of the individual ABMs. As described in more detail below, the ABMs can be arranged differently, depending on the nature of the molecules that form the ABMs (e.g., immunoglobulins, antibody fragments, and alternative scaffolds). The ABMs can also be present in different quantities, to vary the valency of the interactions and/or provide improved control over effector cell activation.

FIG. 10 provides a schematic of a multispecific immunomodulatory antigen-binding construct of this disclosure (i.e., a MIAC; 101). The MIAC comprise two antigen-binding modules (ABMs; 102 and 104). Antigen-binding module 1 (ABM1; 102) comprises a cancer cell-specific antigen-binding site. Antigen-binding module 3 (ABM2; 104) comprises a binding site with antagonist activity toward an inhibitory receptor expressed by an effector cell. This schematic is for purposes of illustrating the components of a MIAC provided herein, and is not limiting with respect to the arrangement or number of the individual ABMs. As described in more detail below, the ABMs can be arranged differently, depending on the nature of the molecules that form the ABMs (e.g., immunoglobulins, antibody fragments, and alternative scaffolds). The ABMs can also be present in different quantities, to vary the valency of the interactions and/or provide improved control over effector cell activation.

FIGS. 11A-11D provides schematics of certain illustrative embodiments of MIACs provided herein. These illustrative embodiments are assembled from an IgG and two scFvs. FIGS. 11A and 11C provide schematics of embodiments where two scFvs are attached to the C-termini of the IgG heavy chains. FIGS. 11B and 11D provide schematics of embodiments where two scFvs are attached to the C-termini of the IgG light chains. In FIGS. 11A-11D, ABM1 is designated as 201, ABM2 is designated as 202, and ABM3 is designated as 203.

FIGS. 12A-12B provides schematics of certain illustrative embodiments of MIACs provided herein. These illustrative embodiments are assembled from an IgG and two scFvs. FIGS. 12A and 12B provide schematics of embodiments where one scFv is attached to the C-terminus of an IgG light chain and one scFv is attached to the C-terminus of an IgG heavy chain. In FIGS. 12A-12B, ABM1 is designated as 201, ABM2 is designated as 202, and ABM3 is designated as 203.

FIGS. 13A-13D provides schematics of further illustrative embodiments of MIACs provided herein. The illustrative embodiments provided in FIGS. 13A-13D show MIACs in which the activation of an effector cell can be modulated by varying the number of binding sites for the activating or inhibitory receptors. These illustrative embodiments are assembled from an IgG and three scFvs. FIG. 13A provides a schematic of an embodiment where one ABM2 (202) is attached to the C-terminus of one IgG heavy chain, one ABM2 (202) is attached to the C-terminus of the other IgG heavy chain, and one ABM2 (202) is attached to the C-terminus of one IgG light chain. FIG. 13B provides a schematic of an embodiment where one ABM2 (202) is attached to the C-terminus of one IgG light chain, one ABM2 (202) is attached to the C-terminus of the other IgG light chain, and one ABM2 (202) is attached to the C-terminus of one IgG heavy chain. FIG. 13C provides a schematic of an embodiment where one ABM3 (203) is attached to the C-terminus of one IgG light chain, one ABM3 (203) is attached to the C-terminus one IgG heavy chain, and one ABM3 (203) is attached to the C-terminus of the other IgG heavy chain FIG. 13D provides a schematic of an embodiment where one ABM3 (203) is attached to the C-terminus of one IgG light chain, one ABM3 (203) is attached to the C-terminus one IgG heavy chain, and one ABM3 (203) is attached to the C-terminus of the other IgG light chain.

FIGS. 14A-14B provides schematics of further illustrative embodiments of MIACs provided herein. These illustrative embodiments are assembled from an IgG and four scFvs. FIG. 14A provides a schematic of an embodiment where ABM2 (202) is attached to the C-termini of all heavy and light chains of the IgG molecule. FIG. 14B provides a schematic of an embodiment where ABM3 (203) is attached to the C-termini of all heavy and light chains of the IgG molecule. In FIGS. 14A-14B, ABM1 is designated as 201, ABM2 is designated as 202, and ABM3 is designated as 203.

FIGS. 15A-15B provides schematics of further illustrative embodiments of MIACs provided herein, wherein ABM1 is an scFv and ABM2 or ABM3 are each an scFv and an IgG. In FIGS. 15A and 15B, ABM1 is designated as 201, ABM2 is designated as 202, and ABM3 is designated as 203.

FIGS. 16A-16B provides schematics of further illustrative embodiments MIACs provided herein, wherein each of ABM1, ABM2, and ABM3 is an scFv. In FIG. 16A-16B, ABM1 is designated as 201, ABM2 is designated as 202, and ABM3 is designated as 203.

FIGS. 17A-17B provides schematics of further illustrative embodiments of the MIACs provided herein. In FIG. 17A-17B, two ABM1 (201) scFvs are attached to the C-terminus of the heavy chains of an IgG-like molecule. The N-terminal region of the IgG-like molecule comprises V_{H}-V_{L} regions forming two ABM2 binding sites (202), and V_{H}-V_{L} regions forming two ABM3 binding sites (203). In the embodiment depicted in FIG. 17A, the ABM3 binding sites are the most N-terminal ABMs formed by the IgG-like molecule. In the embodiment depicted in FIG. 17B, the ABM2 binding sites are the most N-terminal ABMs formed by the IgG-like molecule.

FIGS. 18A-18B provides schematics of further illustrative embodiments of the MIACs provided herein. In FIG. 18A-18B, two ABM1 (201) scFvs are attached to the C-terminus of the heavy chains of an IgG-like molecule. In FIG. 18A, the N-terminal region of the IgG-like molecule comprises V_{H}-V_{L} regions forming four ABM2 binding sites (202). In FIG. 18A, the N-terminal region of the IgG-like molecule comprises V_{H}-V_{L} regions forming four ABM3 binding sites (203).

FIG. 19 provides an embodiment where ABM1 is an IgG with two binding sites for CD30. ABM2 is an scFv with an agonistic binding site for CD137, and ABM3 is an scFv with an antagonistic binding site for PD-1. The ABM 2 and 3 scFvs are attached to the C-termini of the heavy chains of the IgG forming ABM1. However, as described throughout this disclosure, one or more of the scFvs can also be attached to the N-termini of the heavy chains and/or the C- or N-termini of the light chains.

FIG. 20 provides an embodiment where two ABM1 scFvs are attached to the C-terminus of the heavy chains of an IgG-like molecule. The N-terminal region of the IgG-like molecule comprises V_{H}-V_{L} regions forming two ABM2 binding sites, and V_{H}-V_{L} regions forming two ABM3 binding sites. In this MIAC, the ABM3 binding sites are the most N-terminal ABMs formed by the IgG-like molecule.

FIGS. 21A-C provide schematics for four exemplary MIAC constructs (PID7, PID92, PID128, and PID130) that were engineered, expressed, and purified for use in functional in vitro assays. PID7 (FIG. 21A) is a bispecific MIAC comprised of two ABM1 scFvs with specificity for the Her2 tumor-antigen fused to the heavy chain c-termini of an IgG1 ABM3 that recognizes PD-1. PID92 (FIG. 21B) is a bispecific MIAC comprised of two ABM1 scFvs against Her2 fused to the heavy chain c-termini of an IgG1 ABM2 against CD137. PID128 (FIG. 21C) is a bispecific MIAC comprised of two ABM1 scFvs recognizing Her2 fused to the heavy chain c-termini of an IgG1 ABM2 against CD3. PID130 (FIG. 21D) is a trispecific MIAC comprised of two ABM1 scFvs against Her2 fused to the heavy chain c-termini of an IgG1 ABM2 against CD3 and two ABM3 scFvs recognizing PD-1 that are fused to the light chain c-termini of the IgG. Sequence information for these exemplary MIACs are provided in Section 16.

FIG. 22 contains three SEC chromatographs displaying the POI (peak of interest) of exemplary MIAC proteins PID3 (monospecific α-CD3), PID128 (bispecific α-Her2/α-CD3), and PID130 (trispecific α-Her2/α-CD3/α-PD-1).

FIG. 23 contains ForteBio Octet® binding sensograms from two exemplary MIAC proteins, PID7 (α-Her2/α-PD-1) and PID92 (α-Her2/α-CD137), demonstrating that antigen binding activity is retained in the MIAC format. Measured affinities from these examples and other exemplary MIACs are displayed in Table E.

FIG. 24 shows the expression of the Her2 tumor antigen and the immunoinhibitory ligand PD-L1 on the surface of four different cancer cell lines (A431, MDA-MB-453, JIMT1, NCI-H441) as measured using flow cytometry.

FIG. 25 shows concentration-dependent tumor cell binding activity of exemplary Her2-targeted MIAC proteins. Binding activity of the MIACs was compared against monospecific anti-Her2 (trastuzumab, as a positive control) and monospecific anti-CD3 (negative control).

FIG. 26 contains two graphs showing levels of CD25 expression (by flow cytometry) on CD4+ and CD8+ T cells that were co-cultured with Her2+ JIMT1 tumor cells in the presence of increasing concentrations of the exemplary bispecific α-Her2/α-CD137 MIAC (PID92). Effects of the MIAC were compared against an equivalent concentration of combined monospecific α-Her2 and α-CD137 antibodies.

FIG. 27 contains two graphs showing the proliferation of CD4+ and CD8+ T cells (as determined by CFSE dilution) that were co-cultured with Her2+ JIMT1 tumor cells in the presence of increasing concentrations of the exemplary bispecific α-Her2/α-CD137 MIAC (PID92). Effects of the MIAC were compared against an equivalent concentration of combined monospecific α-Her2 and α-CD137 antibodies.

FIG. 28 contains two graphs showing IFN-γ and TNF-α cytokine induction (as determined by Luminex) from human primary T cells that were co-cultured with Her2+ JIMT1 tumor cells in the presence of increasing concentrations of the exemplary bispecific α-Her2/α-CD137 MIAC (PID92). Effects of the MIAC were compared against an equivalent concentration of combined monospecific α-Her2 and α-CD137 antibodies.

FIG. 29A-B shows proliferation (FIG. 29A) and induction of CD25 expression (FIG. 29B), as determined by flow cytometry, in human primary T cells that were co-cultured with Her2+ JIMT1 tumor cells in the presence of increasing concentrations of the exemplary bispecific α-Her2/α-CD3 MIAC (PID128). Effects of the MIAC were compared against equivalent concentrations of combined monospecific α-Her2 and α-CD3.

FIG. 30 contains two graphs showing IFN-γ and TNF-α cytokine induction (as determined by Luminex) from human primary T cells that were co-cultured with Her2+ JIMT1 tumor cells in the presence of increasing concentrations of the exemplary bispecific α-Her2/α-CD3 MIAC (PID128). Effects of the MIAC were compared against equivalent concentrations of combined monospecific α-Her2 and α-CD3.

FIG. 31 shows levels of granzyme B (as determined by Luminex) in co-cultures of human primary T cell and Her2+ JIMT1 tumor cells that were exposed to increasing concentrations of the exemplary bispecific α-Her2/α-CD3 MIAC (PID128). Effects of the MIAC were compared against equivalent concentrations of combined monospecific α-Her2 and α-CD3.

FIG. 32A-B shows proliferation (FIG. 32A) and induction of CD25 expression (FIG. 32B), as determined by flow cytometry, in human primary T cells that were co-cultured with Her2+ JIMT1 tumor cells in the presence of increasing concentrations of the exemplary trispecific α-Her2/α-CD3/α-PD-1 MIAC (PID130). Effects of the MIAC were compared against equivalent concentrations of combined monospecific α-Her2, α-CD3, and α-PD-1.

FIG. 33 contains two graphs showing IFN-γ and TNF-α cytokine induction (as determined by Luminex) from human primary T cells that were co-cultured with Her2+ JIMT1 tumor cells in the presence of increasing concentrations of the exemplary trispecific a-Her2/α-CD3/α-PD-1 MIAC (PID130). Effects of the MIAC were compared against equivalent concentrations of combined monospecific α-Her2, α-CD3, and α-PD-1.

FIG. 34 shows levels of granzyme B (as determined by Luminex) in co-cultures of human primary T cell and Her2+ JIMT1 tumor cells that were exposed to increasing concentrations of the exemplary trispecific α-Her2/α-CD3/α-PD-1 MIAC (PID130). Effects of the MIAC were compared against equivalent concentrations of combined monospecific a-Her2, α-CD3, and α-PD-1.

FIG. 35 shows the results of an in vitro cellular cytotoxicity assay in which CD3+ T cells were co-cultured with Her2+BT474 human cancer cells in the presence of increasing concentrations of the exemplary bispecific α-Her2/α-CD3 MIAC (PID128). Effects of the MIAC were compared against equivalent concentrations of combined α-Her2 and α-CD3 monospecific antibodies. T cell killing of tumor cells was assayed using a CytoTox 96® kit (Promega) to measure lactate dehydrogenase release from lysed cells.

FIG. 36 contains two graphs showing IFN-γ and granzyme B induction (as determined by Luminex) in CD3+ T cell/BT474 tumor cell co-cultures that were exposed to increasing concentrations of the exemplary bispecific α-Her2/α-CD3 MIAC (PID128). Effects of the MIAC were compared against equivalent concentrations of combined α-Her2 and α-CD3 monospecific antibodies.

### DETAILED DESCRIPTION

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value±10%, ±5%, or ±1%.

The term “comprising” is used herein in its most inclusive and open-ended sense, and does not exclude any additional elements or method steps recited in a claim. For examples, a MIAC comprising an ABM1, an ABM2, and an ABM3 can also contain any other ABM (e.g., ABM4) or multiple copies or versions of ABM1, ABM2, and/or ABM3. The MIAC can also comprise any other element that is not an ABM. Similarly, an ABM comprising two immunoglobulin variable domains can also contain three, four, five, six, seven, eight, or more than eight immunoglobulin domains.

The term “consisting of” is used herein to exclude any element, step, or ingredient that is not recited in a clause immediately following this transitional phrase, except for impurities ordinarily associated with the claimed subject matter.

The term “consisting essentially of” is used herein to limit the scope of a claim to the specified materials or steps and those that do not materially affect the basic and novel characteristics of the claimed invention.

The term “antigen-binding module 1” or “ABM1,” refers to an antigen-binding module that binds specifically to an antigen expressed by a cancer cell.

The term “antigen-binding module 2” or “ABM2,” refers to an antigen-binding module that binds specifically to an activating receptor expressed by an effector cell such as an effector immune cell, wherein the binding of ABM2 to the activating receptor of the effector cell agonizes the activating receptor.

The term “antigen-binding module 3” or “ABM3,” refers to an antigen-binding module that binds specifically to an inhibitory receptor expressed by an effector cell such as an effector immune cell, wherein the binding of ABM3 to the inhibitory receptor of the effector cell antagonizes the inhibitory receptor.

The term “antigen-binding module 4” or “ABM4,” refers to an antigen-binding module that binds to an Fc receptor on an effector cell. In some embodiments, ABM4 agonizes the Fc receptor.

The terms “agonist” and “agonize,” when used to refer to the biological activity of ABM2, indicate that ABM2 binds its receptor target on an effector cell and activates that receptor to induce a biological response in the effector cell through the receptor.

The terms “antagonist” and “antagonize,” when used to refer to the biological activity of ABM3 indicate that ABM3 binds its receptor target on the effector cell and blocks or inhibits activation of that receptor (e.g., by its endogenous ligands) to prevent induction of a biological response in the effector cell through the receptor.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, Pa. Briefly, each heavy chain typically comprises a heavy chain variable region (V_{H}) and a heavy chain constant region (C_{H}). The heavy chain constant region typically comprises three domains, C_{H1}, C_{H2}, and C_{H3}. Each light chain typically comprises a light chain variable region (V_{L}) and a light chain constant region. The light chain constant region typically comprises one domain, abbreviated C_{L}.

The term “antibody” describes a type of immunoglobulin molecule and is used herein in its broadest sense. An antibody specifically includes intact antibodies (e.g., intact immunoglobulins), and antibody fragments such as antigen binding fragments of an antibody. Antibodies comprise at least one antigen-binding domain. One example of an antigen-binding domain is an antigen binding domain formed by a V_{H}-V_{L} dimer. Antibodies can be described by the antigen to which they specifically bind. For example, an NKG2D antibody, or anti-NKG2D antibody, is an antibody that specifically binds to the receptor NKG2D. An antibody can be further described by its activity. For example, an agonizing NKG2D antibody is an antibody that binds to the receptor NKG2D and agonizes it.

The V_{H} and V_{L} regions can be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and confer antigen specificity and binding affinity to the antibody. See Kabat et al., Sequences of Proteins of Immunological Interest 5th ed. (1991) Public Health Service, National Institutes of Health, Bethesda, Md., incorporated by reference in its entirety.

The light chain from vertebrate species can be assigned to one of two types, called kappa and lambda, based on the sequence of the constant domain.

The heavy chain from vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

Unless otherwise specified, the numbering scheme used for identification of a particular CDR herein is the Kabat/Chothia numbering scheme. Where the residues encompassed by these two numbering schemes diverge, the numbering scheme is specified as either Kabat or Chothia.

TABLE 1
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy or light chain constant region (e.g., as reported in Kabat et al., supra). Unless stated otherwise, the EU numbering scheme is used to refer to residues in antibody heavy chain and light chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, scFv-Fc fragments, and single-domain antibodies.

“Fv” fragments comprise a non-covalently-linked dimer of one heavy chain variable domain and one light chain variable domain.

“Fab” fragments comprise, in addition to the heavy and light chain variable domains of the Fv fragment, the constant domain of the light chain and the first constant domain (C_{H1}) of the heavy chain. Fab fragments can be generated, for example, recombinantly or by papain digestion of a full-length antibody.

“F(ab′)₂” fragments contain two Fab fragments joined, near the hinge region, by disulfide bonds. F(ab′)₂ fragments can be generated, for example, recombinantly or by pepsin digestion of an intact antibody, which removes most of the Fc region while leaving part of the hinge region intact. The F(ab′)₂ fragment can be dissociated (into two F(ab′) molecules) by treatment with a reducing agent such as β-mercaptoethanol.

“Single-chain Fv” or “sFv” or “scFv” fragments comprise a V_{H} domain and a V_{L} domain in a single polypeptide chain. The V_{H} and V_{L} are generally linked by a peptide linker. See Plückthun A. (1994). Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety. In some embodiments, the linker can be a single amino acid. In some embodiments, the linker can be a chemical bond. “scFv-Fc” fragments comprise an scFv attached to an Fc domain. For example, an Fc domain can be attached to the C-terminal of the scFv. The Fc domain can follow the V_{H} or V_{L}, depending on the orientation of the variable domains in the scFv (i.e., V_{H}-V_{L} or V_{L}-V_{H}). The Fc domain can be any suitable Fc domain known in the art or described herein. In some cases, the Fc domain is an IgG1 Fc domain.

“Single-domain antibodies” are antibody fragments comprising a single monomeric immunoglobulin variable domain. See Holt et al., Trends in Biotechnol., 2003, 21:484-490, incorporated by reference in its entirety. Single-domain antibodies can comprise either a single heavy chain or a single light chain. An example of a single light chain antibody is provided in Masat et al., Proc. Natl. Acad. Sci. USA, 1994, 91:893-896.

The term “dual variable domain immunoglobulin” or “DVD-Ig™” refers to multivalent and multispecific binding proteins as described, for example, in DiGiammarino et al., Methods Mol. Biol., 2012, 899:145-156, and in U.S. Pat. Nos. 7,612,181; 8,258,268; 8,586,714; 8,716,450; 8,722,855; 8,735,546; and 8,822,645; each of which is incorporated by reference in its entirety.

The term “heavy chain antibody” refers to an antibody which comprises at least two heavy chains and lacks light chains. See Harmesen et al., Applied Microbiology and Biotechnology, 77:13-22, 2007; and Hamers-Casterman et al., Nature, 1993, 363:446-448; each of which is incorporated by reference in its entirety.

The term “monoclonal antibody” refers to an antibody from a population of substantially homogeneous antibodies. A population of substantially homogeneous antibodies comprises antibodies that are substantially similar and that bind the same epitope(s), except for variants that can normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of yeast clones, phage clones, bacterial clones, mammalian cell clones, hybridoma clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies can also comprise residues that are not found in either the recipient antibody or the donor antibody. Such modifications can be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

The term “alternative scaffold” refers to a molecule in which one or more regions can be diversified to produce molecules with specificities and affinities that are similar to those of antibodies. Exemplary alternative scaffolds include those derived from fibronectin (e.g., Adnectins™), the β-sandwich (e.g., iMab), lipocalin (e.g., Anticalins®), EETI-II/AGRP, BPTI/LACI-D1/ITI-D2 (e.g., Kunitz domains), thioredoxin peptide aptamers, protein A (e.g., Affibody), ankyrin repeats (e.g., DARPins), gamma-B-crystallin/ubiquitin (e.g., Affilins), CTLD₃ (e.g., Tetranectins), and (LDLR-A module) (e.g., Avimers). Additional information on alternative scaffolds is provided in Binz et al., Nat. Biotechnol., 2005 23:1257-1268; and Skerra, Current Opin. in Biotech., 2007 18:295-304, each of which is incorporated by reference in its entirety.

The term “operably linked” indicates that the MIAC portions (e.g., one or more ABMs and/or Fc) are arranged so that they function in concert for their intended purposes.

An “isolated MIAC” is one that has been separated and/or recovered from a component of its production environment. Components of the environment can include cells, media, and other proteinaceous or nonproteinaceous materials, such as nucleic acids. In some embodiments, an isolated MIAC is purified to a degree sufficient to obtain at least 15 residues of N-terminal or internal amino acid sequence, for example by use of a spinning cup sequenator. In some embodiments, an isolated MIAC is purified to homogeneity as analyzed by gel electrophoresis (e.g., SDS-PAGE) under reducing or nonreducing conditions, with detection by Coomassie blue or silver stain. In some embodiments, an isolated MIAC includes a MIAC in situ within recombinant cells that produce the MIAC. In some embodiments, an isolated MIAC is prepared by at least one purification step.

In some embodiments, an isolated MIAC is purified to at least 80%, 85%, 90%, 95%, or 99% by weight. In some embodiments, an isolated MIAC is provided as a solution comprising at least 85%, 90%, 95%, 98%, 99% to 100% by weight of a MIAC, the remainder of the weight comprising the weight of other solutes dissolved in the solvent.

“Affinity” refers to the strength of the sum total of non-covalent interactions between a single binding site of a molecule (e.g., an antigen-binding module of a MIAC) and its binding partner (e.g., the antigen). Unless indicated otherwise, as used herein, “binding affinity” refers to intrinsic binding affinity, which reflects a 1:1 interaction between members of a binding pair (e.g., antigen-binding module and antigen). The affinity of a molecule X for its partner Y can generally be represented by the dissociation constant (K_{D}) Affinity can be measured by methods known in the art, for example by using surface plasmon resonance (SPR) technology (e.g., Biacore® instruments) or bio-layer interferometry (e.g., ForteBio® instruments).

With regard to the binding of an ABM to a target molecule, the terms “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that is similar to the target, such as an excess of non-labeled target. In that case, specific binding is indicated if the binding of the labeled target to a probe is competitively inhibited by the excess non-labeled target.

The term “k_{d}” (sec⁻¹), as used herein, refers to the dissociation rate constant of a particular ABM-antigen interaction. This value is also referred to as the k_{off} value.

The term “kₐ” (M⁻¹×sec⁻¹), as used herein, refers to the association rate constant of a particular ABM-antigen interaction. This value is also referred to as the kₒₙ value.

The term “K_{D}” (M), as used herein, refers to the dissociation equilibrium constant of a particular ABM-antigen interaction. K_{D}=k_{d}/kₐ.

The term “K_{A}” (M⁻¹), as used herein, refers to the association equilibrium constant of a particular ABM-antigen interaction. K_{A}=kₐ/k_{d}.

An “affinity matured” ABM is one with one or more alterations (e.g., in one or more CDRs or FRs) that result in an improvement in the affinity of the ABM for its antigen, compared to a parent ABM which does not possess the alteration(s). In one embodiment, an affinity matured ABM has nanomolar or picomolar affinity for the target antigen Affinity matured ABMs can be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. USA., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

When used herein in the context of two or more ABMs, the term “competes with” or “cross-competes with” indicates that the two or more ABMs compete for binding to an antigen. In one exemplary assay, the antigen is coated on a plate and allowed to bind a first ABM, after which a second, labeled ABM is added. If the presence of the first ABM reduces binding of the second ABM, then the ABMs compete. The term “competes with” also includes combinations of ABMs where one ABM reduces binding of another ABM, but where no competition is observed when the ABMs are added in the reverse order. However, in some embodiments, the first and second ABMs inhibit binding of each other, regardless of the order in which they are added. In some embodiments, one ABM reduces binding of another ABM to its antigen by at least 50%, at least 60%, at least 70%, at least 80%, or at least 90%.

The term “epitope” means a portion of an antigen capable of specific binding to an ABM. Epitopes frequently consist of surface-accessible amino acid residues and/or sugar side chains and can have specific three dimensional structural characteristics, as well as specific charge characteristics. Conformational and non-conformational epitopes are distinguished in that the binding to the former but not the latter is lost in the presence of denaturing solvents. An epitope can comprise amino acid residues that are directly involved in the binding, and other amino acid residues, which are not directly involved in the binding. The epitope to which an ABM binds can be determined using known techniques for epitope determination such as, for example, testing for ABM binding to antigen variants with different point-mutations.

The term “valency,” when used to describe an ABM, refers to the number of antigen recognition (binding) sites in an ABM. Each antigen recognition site specifically recognizes, and is therefore capable of binding, one antigen or epitope on an antigen. When an ABM comprises more than one antigen recognition site (e.g., when an ABM is an IgG, which has two antigen recognition sites in its variable regions), each antigen recognition site can specifically recognize the same or different antigens. However, in some embodiments, each antigen recognition site in an ABM specifically recognizes the same antigen.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, or CLUSTAL OMEGA software. In some embodiments, alignment is performed using the CLUSTAL OMEGA software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of one or more amino acids with one or more chemically or functionally similar amino acids. Conservative substitution tables providing similar amino acids are well known in the art. Polypeptide sequences having such substitutions are known as “conservatively modified variants.” Such conservatively modified variants are in addition to and do not exclude polymorphic variants, interspecies homologs, and alleles. By way of example, the groups of amino acids provided in Tables 2-4 are considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Additional selected groups of amino acids that
are considered conservative substitutions for
one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions can be found, for example, in Creighton, Proteins: Structures and Molecular Properties 2nd ed. (1993) W. H. Freeman & Co., New York, N.Y. An ABM generated by making one or more conservative substitutions of amino acid residues in a parent ABM is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V). In some embodiments, the term “amino acid” also includes non-natural amino acids. Any suitable non-natural amino acid can be used. In some embodiments, the non-natural amino acids comprise a reactive moiety for conjugation of an agent to a MIAC.

“Treating” or “treatment” of any disease or disorder refers, in certain embodiments, to ameliorating a disease or disorder that exists in a subject. In another embodiment, “treating” or “treatment” includes ameliorating at least one physical parameter, which can be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of a MIAC that when administered to a subject is effective to treat a disease or disorder.

As used herein, the term “subject” means a mammalian subject. Exemplary subjects include, but are not limited to humans, monkeys, dogs, cats, mice, rats, cows, horses, camels, goats and sheep. In certain embodiments, the subject is a human. In some embodiments, the subject has or is suspected to have a disease or condition that can be treated with a MIAC provided herein. In some aspects, the disease or condition is a cancer. In some embodiments, the subject is a human with a cancer that can be treated with a MIAC provided herein. In some embodiments, the subject is a human that is suspected to have cancer that can be treated with a MIAC provided herein.

### 2. Multispecific Immunomodulatory Antigen-Binding Constructs (MIACs)

2.1. Multispecific Immunomodulatory Antigen-Binding Constructs (MIACs) that Target Cancer Cells and Simultaneously Modulate Activating and Inhibitory Receptors on the Same Effector Cell

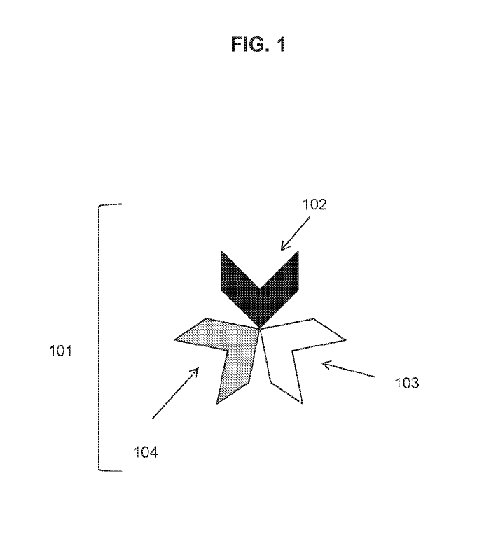

One aspect of the MIAC concept is illustrated in FIG. 1, which shows a MIAC comprising, consisting essentially of, or consisting of three ABMs. ABM1 comprises a cancer cell-specific antigen-binding site. ABM2 comprises a binding site with agonist activity toward an activating receptor expressed by an effector cell. ABM3 comprises a binding site with antagonist activity toward an inhibitory receptor expressed by an effector cell.

The illustrative embodiment provided in FIG. 1 provides a MIAC concept in which the MIAC targets a cancer cell and simultaneously modulates activating and inhibitory receptors on the same effector cell. As described in more detail elsewhere in this disclosure, the number of binding sites for each component of the MIAC can be varied by selecting the type of molecule that forms the ABM, or by varying the number of each ABM included in the MIAC. For example, selecting a single IgG as an ABM will yield two binding sites for the targeted antigen. On the other hand, selecting a single scFv as an ABM will yield a single binding site for the targeted antigen.

In some embodiments, the MIACs provided herein comprise, consist essentially of, or consist of: (a) an antigen-binding module 1 (ABM1) that binds specifically to an antigen expressed by a cancer cell; (b) an antigen-binding module 2 (ABM2) that binds specifically to an activating receptor expressed by an effector cell; and (c) an antigen-binding module 3 (ABM3) that binds specifically to an inhibitory receptor expressed by the effector cell.

The binding of ABM2 to the activating receptor of the effector cell agonizes the activating receptor, thereby promoting activation of the effector cell by promoting the transduction of activating signals through the activating receptor. In some embodiments, transduction of activating signals through the activating receptor induces a response from the effector cell selected from proliferation, cytotoxic activity against a cancer cell, secretion of cytokines (e.g., IL-2 and interferon gamma), upregulation of LAMP-1, downregulation of CD16, upregulation of CD69, and upregulation of KLRG1.

The binding of ABM3 to the inhibitory receptor of the effector cell antagonizes the inhibitory receptor, thereby promoting activation of the effector cell by blocking the transduction of inhibitory signaling through the inhibitory receptor. In some embodiments, blocking the transduction of inhibitory signaling through the inhibitory receptor induces a response from the effector cell selected from proliferation, cytotoxic activity against a cancer cell, secretion of cytokines (e.g., IL-2 and interferon gamma), upregulation of LAMP-1, downregulation of CD16, upregulation of CD69, and upregulation of KLRG1.

2.2. Multispecific Immunomodulatory Antigen-Binding Constructs (MIACs) that Target Cancer Cells and Agonize Activating Receptors Expressed by Effector Cells

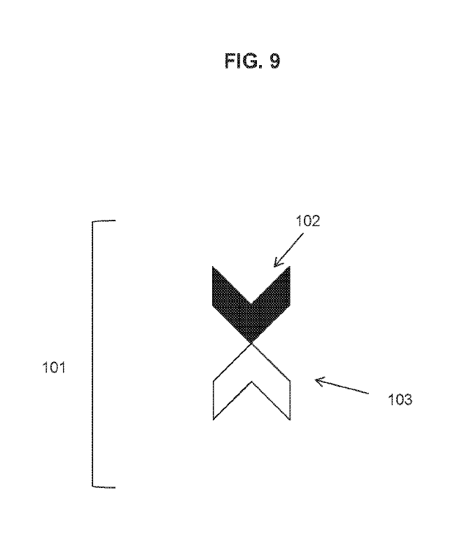

Another aspect of the MIAC concept is illustrated in FIG. 9, which shows a MIAC comprising, consisting essentially of, or consisting of two ABMs. ABM1 comprises a cancer cell-specific antigen-binding site. ABM2 comprises a binding site with agonist activity toward an activating receptor expressed by an effector cell.

The illustrative embodiment provided in FIG. 9 provides a MIAC concept in which the MIAC targets a cancer cell and modulates an activating receptor on an effector cell. As described in more detail elsewhere in this disclosure, the number of binding sites for each component of the MIAC can be varied by selecting the type of molecule that forms the ABM, or by varying the number of each ABM included in the MIAC. For example, selecting a single IgG as an ABM will yield two binding sites for the targeted antigen. On the other hand, selecting a single scFv as an ABM will yield a single binding site for the targeted antigen.

In some aspects, the MIACs provided herein comprise, consist essentially of, or consist of: (a) an antigen-binding module 1 (ABM1) that binds specifically to an antigen expressed by a cancer cell; and (b) an antigen-binding module 2 (ABM2) that binds specifically to an activating receptor expressed by an effector cell. In this aspect, the MIACs do not comprise an ABM3.

The binding of ABM2 to the activating receptor of the effector cell agonizes the activating receptor, thereby promoting activation of the effector cell by promoting the transduction of activating signals through the activating receptor. In some embodiments, transduction of activating signals through the activating receptor induces a response from the effector cell selected from proliferation, cytotoxic activity against a cancer cell, secretion of cytokines (e.g., IL-2 and interferon gamma), upregulation of LAMP-1, downregulation of CD16, upregulation of CD69, and upregulation of KLRG1.

In some embodiments, when the MIAC comprises and ABM1 and ABM2, but does not comprises an ABM3, ABM2 specifically does not bind certain canonical receptors involved in effector cell activation. In some embodiments, ABM2 does not bind CD3 and does not bind CD28. In some embodiments, ABM2 does not bind CD3. In some embodiments, ABM2 does not bind CD28.

2.3. Multispecific Immunomodulatory Antigen-Binding Constructs (MIACs) that Target Cancer Cells and Antagonize Inhibitory Receptors Expressed by Effector Cells

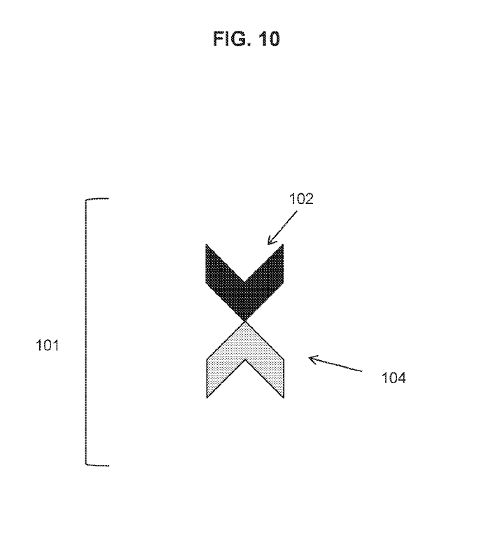

Another aspect of the MIAC concept is illustrated in FIG. 10, which shows a MIAC comprising, consisting essentially of, or consisting of two ABMs. ABM1 comprises a cancer cell-specific antigen-binding site. ABM3 comprises a binding site with antagonist activity toward an inhibitory receptor expressed by an effector cell.

The illustrative embodiment provided in FIG. 10 provides a MIAC concept in which the MIAC targets a cancer cell and modulates an inhibitory receptor on an effector cell. As described in more detail elsewhere in this disclosure, the number of binding sites for each component of the MIAC can be varied by selecting the type of molecule that forms the ABM, or by varying the number of each ABM included in the MIAC. For example, selecting a single IgG as an ABM will yield two binding sites for the targeted antigen. On the other hand, selecting a single scFv as an ABM will yield a single binding site for the targeted antigen.

In some aspects, the MIACs provided herein comprise, consist essentially of, or consist of: (a) an antigen-binding module 1 (ABM1) that binds specifically to an antigen expressed by a cancer cell; and (b) an antigen-binding module 3 (ABM3) that binds specifically to an inhibitory receptor expressed by an effector cell. In this aspect, the MIACs do not comprise an ABM2.

The binding of ABM3 to the inhibitory receptor of the effector cell antagonizes the inhibitory receptor, thereby promoting activation of the effector cell by blocking the transduction of inhibitory signaling through the inhibitory receptor. In some embodiments, blocking the transduction of inhibitory signaling through the inhibitory receptor induces a response from the effector cell selected from proliferation, cytotoxic activity against a cancer cell, secretion of cytokines (e.g., IL-2 and interferon gamma), upregulation of LAMP-1, downregulation of CD16, upregulation of CD69, and upregulation of KLRG1.

2.4. Multispecific Immunomodulatory Antigen-Binding Constructs (MIACs) can Comprise One or More Scaffolds Such as Fc

MIACs can include a scaffold such as Fc. Such a scaffold can be used to operably link each ABM to each other. In certain aspects one or more ABM can include a scaffold such as Fc.

The term the term “Fc” or “Fc domain” or “Fc region” herein is used to define a C-terminal region of an immunoglobulin heavy chain that contains at least a portion of the constant region. The term includes native sequence Fc regions and variant Fc regions. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, Md., 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence.

An Fc domain comprises either a CH3 domain or a CH3 and a CH2 domain. The CH3 domain comprises two CH3 sequences, one from each of the two Fc polypeptides of the dimeric Fc. The CH2 domain comprises two CH2 sequences, one from each of the two Fc polypeptides of the dimeric Fc.

In some aspects, the Fc comprises at least one or two CH3 sequences. In some aspects, the Fc is coupled, with or without one or more linkers, to a first antigen-binding module and/or a second antigen-binding module. In some aspects, the Fc is a human Fc. In some aspects, the Fc is a human IgG or IgG1 Fc. In some aspects, the Fc is a heterodimeric Fc. In some aspects, the Fc comprises at least one or two CH2 sequences.

In some aspects, the Fc comprises one or more modifications in at least one of the CH3 sequences. In some aspects, the Fc comprises one or more modifications in at least one of the CH2 sequences. In some aspects, an Fc is a single polypeptide. In some aspects, an Fc is multiple peptides, e.g., two polypeptides. Fc can be modified, e.g., to include one or more modifications in a CH2 domain and/or a CH3 domain. Such modifications can impact Fc function and binding characteristics such as Fc receptor (FcR) binding. Fc can be modified to block binding to FcR, for example Fc can be modified to include a mutation to block FcR binding such as a mutation at amino acid N297. Fc can be modified to prevent N-linked glycosylation and/or reduce ADCC. An example is shown in the examples section below.

### 3. Antigen-Binding Modules (ABMs)

The ABMs of the MIACs can comprise any suitable antigen-binding molecule. In some embodiments, an ABM comprises a molecule selected from an immunoglobulin, an antibody, an antibody fragment, and/or an alternative scaffold.

In some embodiments, ABM1 is an antibody or antigen-binding fragment thereof. In some embodiments, ABM1 comprises an immunoglobulin molecule selected from an IgG (IgG1, IgG2, IgG3, IgG4), an IgA (IgA1, IgA2), an IgD, an IgE, and an IgM, or a fragment thereof. In some embodiments, ABM1 comprises an Fv fragment, a Fab fragment, a F(ab′)₂ fragment, a Fab′ fragment, an scFv fragment, an scFv-Fc fragment, and a single-domain antibody. In some embodiments, ABM1 is a DVD-Ig™. In some embodiments, ABM1 is a heavy chain antibody.

In some embodiments, ABM1 comprises an alternative scaffold comprising a molecule selected from an Adnectins™, an iMab, an Anticalin®, an EETI-II/AGRP, a Kunitz domain, a thioredoxin peptide aptamer, an Affibody, a DARPins, an Affilins, a Tetranectin, and an Avimer.

In some embodiments, ABM2 is an antibody. In some embodiments, ABM2 comprises an immunoglobulin molecule selected from an IgG (IgG1, IgG2, IgG3, IgG4), an IgA (IgA1, IgA2), an IgD, an IgE, and an IgM, or a fragment thereof. In some embodiments, ABM2 comprises an Fv fragment, a Fab fragment, a F(ab′)₂ fragment, a Fab′ fragment, an scFv fragment, an scFv-Fc fragment, and a single-domain antibody. In some embodiments, ABM2 is a DVD-Ig™. In some embodiments, ABM2 is a heavy chain antibody.

In some embodiments, ABM2 comprises an alternative scaffold comprising a molecule selected from an Adnectins™, an iMab, an Anticalin®, an EETI-II/AGRP, a Kunitz domain, a thioredoxin peptide aptamer, an Affibody, a DARPins, an Affilins, a Tetranectin, and an Avimer.

In some embodiments, ABM3 is an antibody. In some embodiments, ABM3 comprises an immunoglobulin molecule selected from an IgG (IgG1, IgG2, IgG3, IgG4), an IgA (IgA1, IgA2), an IgD, an IgE, and an IgM, or a fragment thereof. In some embodiments, ABM3 comprises an Fv fragment, a Fab fragment, a F(ab′)₂ fragment, a Fab′ fragment, an scFv fragment, an scFv-Fc fragment, and a single-domain antibody. In some embodiments, ABM3 is a DVD-Ig™. In some embodiments, ABM3 is a heavy chain antibody.

In some embodiments, ABM3 comprises an alternative scaffold comprising a molecule selected from an Adnectins™, an iMab, an Anticalin®, an EETI-II/AGRP, a Kunitz domain, a thioredoxin peptide aptamer, an Affibody, a DARPins, an Affilins, a Tetranectin, and an Avimer.

In some embodiments, ABM4 comprises an immunoglobulin molecule selected from an IgG (IgG1, IgG2, IgG3, IgG4), an IgA (IgA1, IgA2), an IgD, an IgE, and an IgM, or a fragment thereof. In some embodiments, ABM4 comprises an Fv fragment, a Fab fragment, a F(ab′)₂ fragment, a Fab′ fragment, an scFv fragment, an scFv-Fc fragment, and a single-domain antibody. In some embodiments, ABM4 is a DVD-Ig™. In some embodiments, ABM4 is a heavy chain antibody.

In some embodiments, ABM4 comprises an alternative scaffold comprising a molecule selected from an Adnectins™, an iMab, an Anticalin®, an EETI-II/AGRP, a Kunitz domain, a thioredoxin peptide aptamer, an Affibody, a DARPins, an Affilins, a Tetranectin, and an Avimer.

The compositions of the ABMs provided herein are illustrative, and any suitable molecule capable of functioning as an ABM can be used in a MIAC provided herein. Molecules capable of functioning as ABMs include proteins, peptides, nucleic acids, lipids, aptamers (peptide and oligonucleotide), and the like that have the binding and functional properties of the ABMs described herein.

3.1. Valency and Immunoglobulin Variable Domains

ABMs provided herein can be characterized in terms of their valency for an antigen. The ABMs can have any suitable valency. In some embodiments, an ABM can be monovalent, divalent, trivalent, tetravalent, or more than tetravalent. A skilled artisan will recognize that valency can be controlled through the selection of the molecules that form the ABMs. For example, an scFv ABM would generally be monovalent, while an IgG ABM would generally be divalent.

In some embodiments, ABM1 is monovalent. In some embodiments, ABM1 is divalent. In some embodiments, ABM1 is trivalent. In some embodiments, ABM1 is tetravalent. In some embodiments, ABM1 has a valency that is greater than tetravalent.

In some embodiments, ABM2 is monovalent. In some embodiments, ABM2 is divalent. In some embodiments, ABM2 is trivalent. In some embodiments, ABM2 is tetravalent. In some embodiments, ABM2 has a valency that is greater than tetravalent.

In some embodiments, ABM3 is monovalent. In some embodiments, ABM3 is divalent. In some embodiments, ABM3 is trivalent. In some embodiments, ABM3 is tetravalent. In some embodiments, ABM3 has a valency that is greater than tetravalent.

In some embodiments, ABM4 is monovalent. In some embodiments, ABM4 is divalent. In some embodiments, ABM4 is trivalent. In some embodiments, ABM4 is tetravalent. In some embodiments, ABM4 has a valency that is greater than tetravalent.

In some embodiments, all ABMs are monovalent. For example, in some aspects, ABM1 is monovalent, ABM2 is monovalent, and ABM3 is monovalent.

In some embodiments, at least one ABM is divalent. For example, in some aspects, ABM1 is divalent, ABM2 is divalent, and ABM3 is divalent. In some aspects, ABM1 is divalent, ABM2 is monovalent, and ABM3 is monovalent. In some aspects, ABM1 is monovalent, ABM2 is divalent, and ABM3 is monovalent. In some aspects, ABM1 is monovalent, ABM2 is monovalent, and ABM3 is divalent.

In some embodiments, the valency can be characterized by the presence of a particular number of immunoglobulin variable domains. In some embodiments, the variable domains are selected from a V_{H} domain and a V_{L} domain.

In some embodiments, ABM1 comprises one immunoglobulin variable domain. In some embodiments, ABM1 comprises two immunoglobulin variable domains. In some embodiments, ABM1 comprises three immunoglobulin variable domains. In some embodiments, ABM1 comprises four immunoglobulin variable domains. In some embodiments, ABM1 comprises more than four immunoglobulin variable domains.

In some embodiments, ABM2 comprises one immunoglobulin variable domain. In some embodiments, ABM2 comprises two immunoglobulin variable domains. In some embodiments, ABM2 comprises three immunoglobulin variable domains. In some embodiments, ABM2 comprises four immunoglobulin variable domains. In some embodiments, ABM2 comprises more than four immunoglobulin variable domains.

In some embodiments, ABM3 comprises one immunoglobulin variable domain. In some embodiments, ABM3 comprises two immunoglobulin variable domains. In some embodiments, ABM3 comprises three immunoglobulin variable domains. In some embodiments, ABM3 comprises four immunoglobulin variable domains. In some embodiments, ABM3 comprises more than four immunoglobulin variable domains.

In some embodiments, each ABM comprises two immunoglobulin variable domains (e.g., two V_{H} domain and a V_{L} domain; two V_{H} domains; or two V_{L} domain). For example, in some aspects, ABM1 comprises two immunoglobulin variable domains, ABM2 comprises two immunoglobulin variable domains, and ABM3 comprises two immunoglobulin variable domains.

In some embodiments, at least one ABM comprises four immunoglobulin variable domains (e.g., two V_{H} domains and two V_{L} domains). For example, in some aspects, ABM1, ABM2, and ABM3 each comprise four immunoglobulin variable domains. In some aspects, ABM1 comprises four immunoglobulin variable domains, ABM2 comprises two immunoglobulin variable domains, and ABM3 comprises two immunoglobulin variable domains. In some aspects, ABM1 comprises two immunoglobulin variable domains, ABM2 comprises four immunoglobulin variable domains, and ABM3 comprises two immunoglobulin variable domains. In some aspects, ABM1 comprises two immunoglobulin variable domains, ABM2 comprises two immunoglobulin variable domains, and ABM3 comprises four immunoglobulin variable domains.

In some embodiments, the valency can be expressed as the ratio of binding sites of one ABM to the number of binding sites of another ABM. Varying this ratio can be beneficial, for example, in tuning the degree of activation of effector cells.

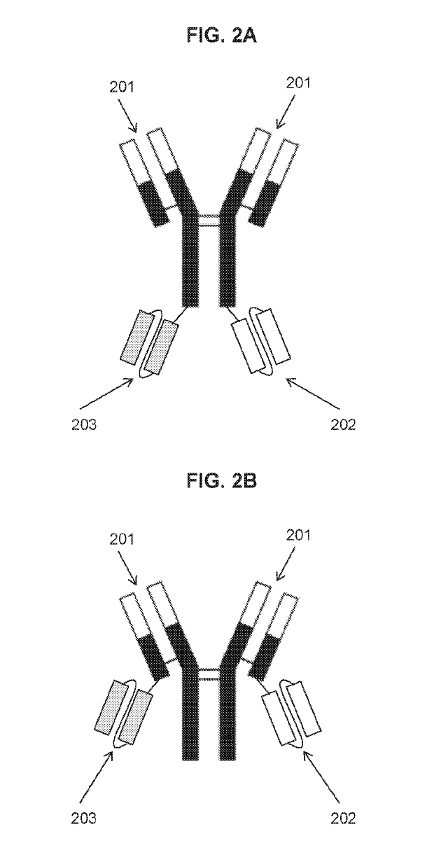

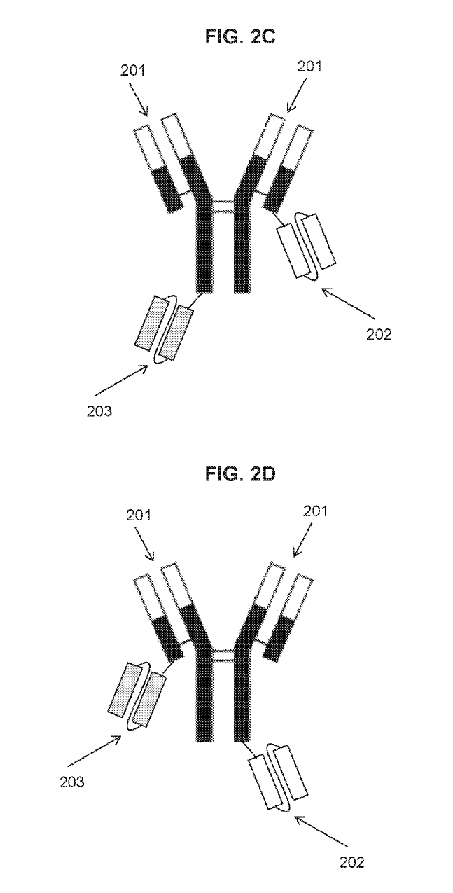

In some embodiments, ABM1, ABM2, and ABM3 binding sites are present in a 2:1:1 ratio. FIGS. 2A-2D show examples of MIACs with binding sites present in this ratio.

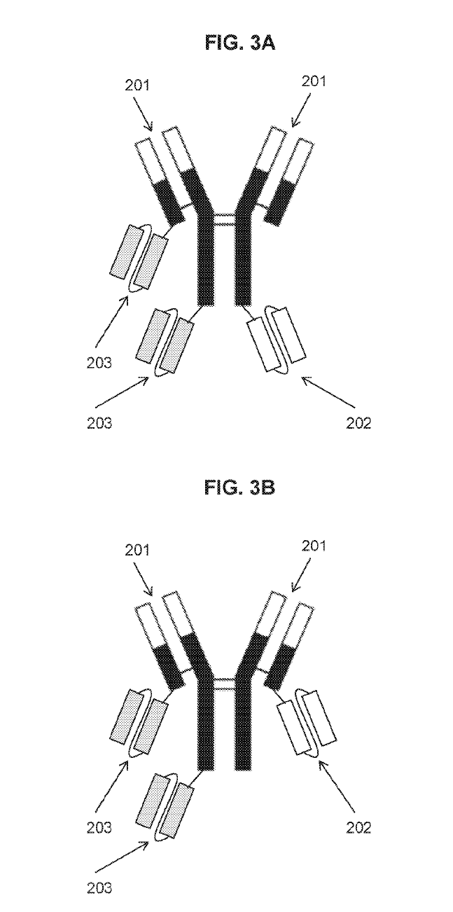

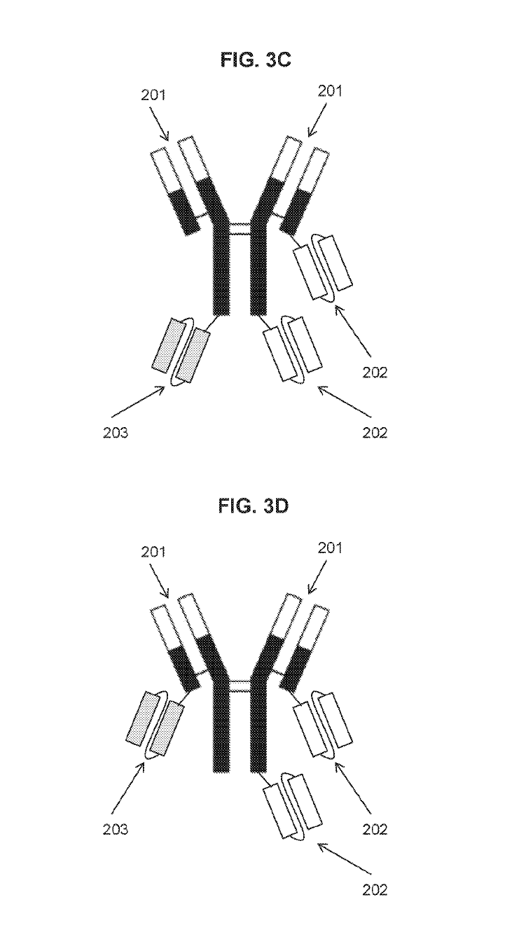

In some embodiments, ABM1, ABM2, and ABM3 binding sites are present in a 2:1:2 ratio. FIGS. 3A-3B show examples of MIACs with binding sites present in this ratio.

In some embodiments, ABM1, ABM2, and ABM3 binding sites are present in a 2:2:1 ratio. FIGS. 3C-3D show examples of MIACs with binding sites present in this ratio.

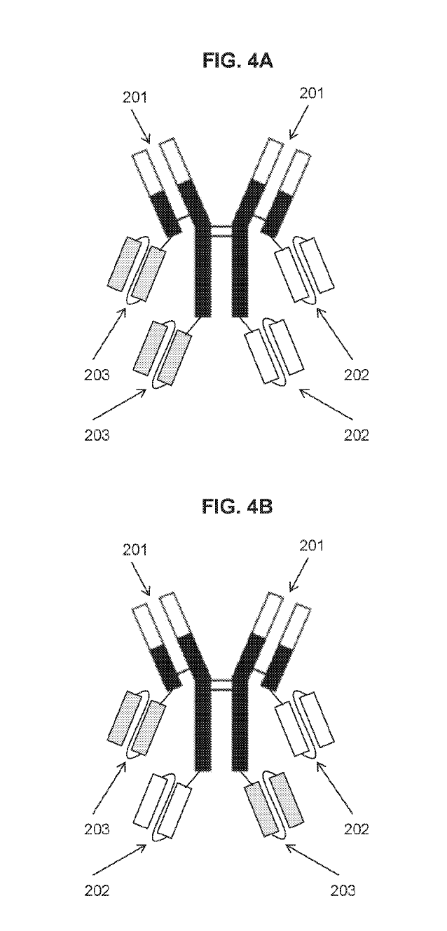

In some embodiments, ABM1, ABM2, and ABM3 binding sites are present in a 2:2:2 ratio. FIGS. 4A-4B show examples of MIACs with binding sites present in this ratio.

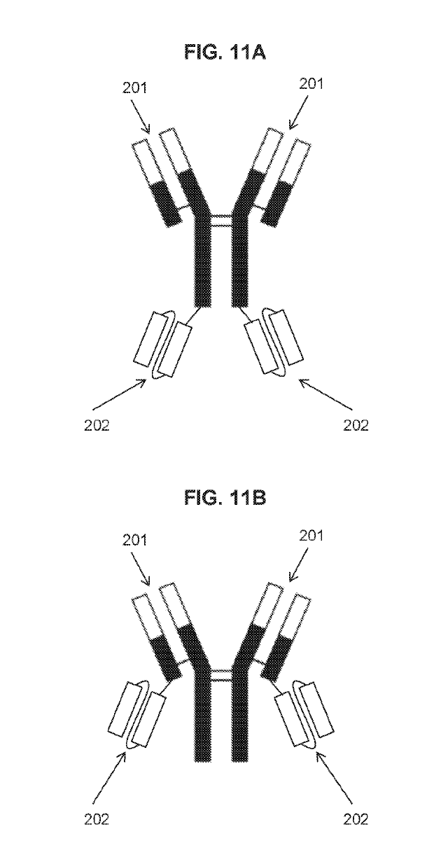

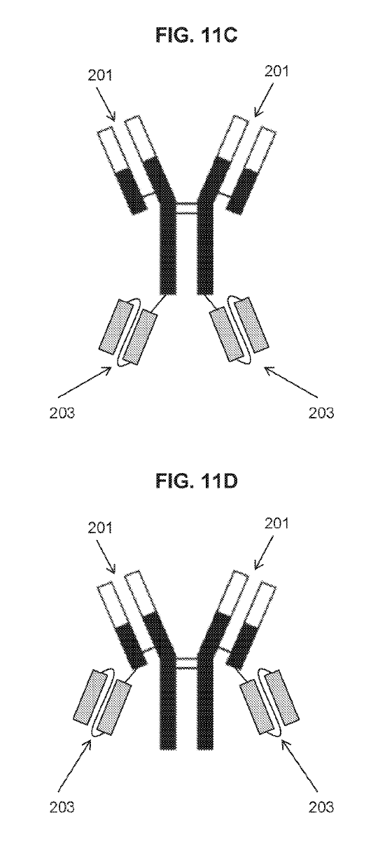

In some embodiments, ABM1, ABM2, and ABM3 binding sites are present in a 2:2:0 ratio. FIGS. 11A-B and 12A show examples of MIACs with binding sites present in this ratio.

In some embodiments, ABM1, ABM2, and ABM3 binding sites are present in a 2:0:2 ratio. FIGS. 11C-D and 12B show examples of MIACs with binding sites present in this ratio.

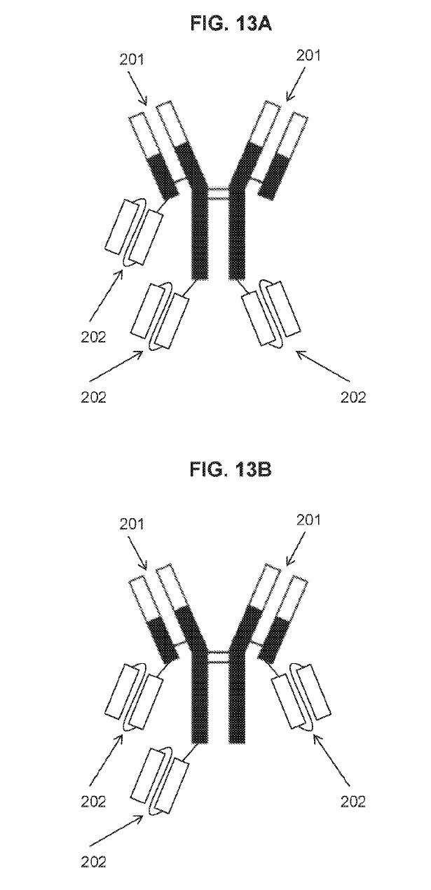

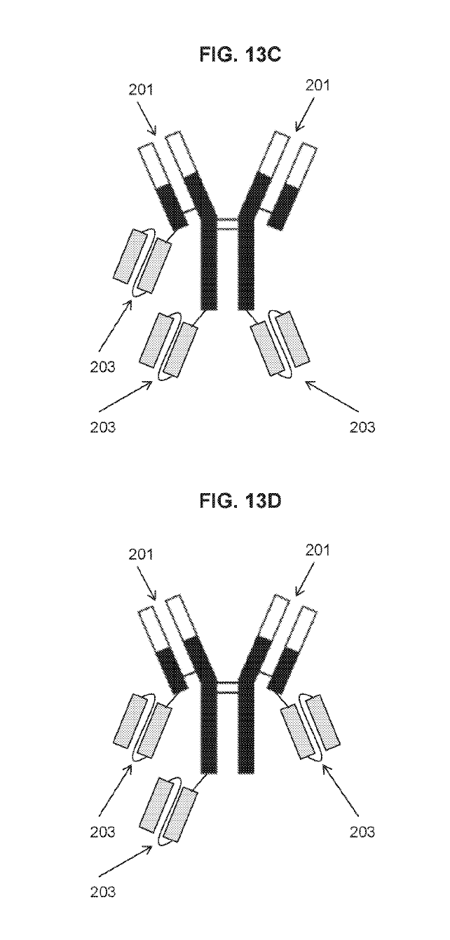

In some embodiments, ABM1, ABM2, and ABM3 binding sites are present in a 2:3:0 ratio. FIGS. 13A-B show examples of MIACs with binding sites present in this ratio.

In some embodiments, ABM1, ABM2, and ABM3 binding sites are present in a 2:0:3 ratio. FIGS. 13C-D show examples of MIACs with binding sites present in this ratio.

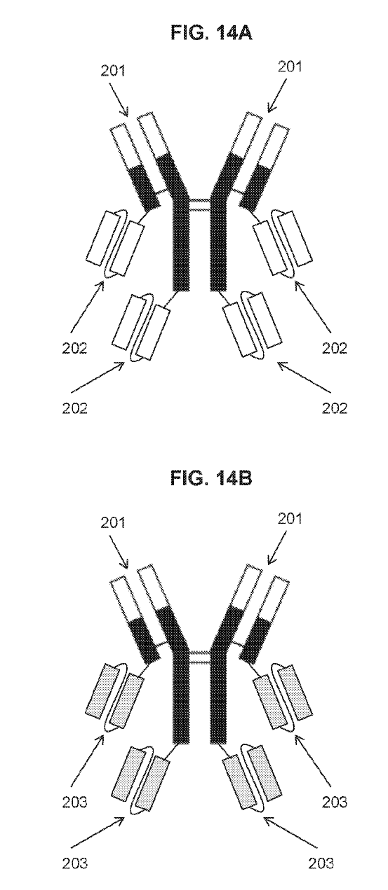

In some embodiments, ABM1, ABM2, and ABM3 binding sites are present in a 2:4:0 ratio. FIG. 14A shows an example of a MIAC with binding sites present in this ratio.

In some embodiments, ABM1, ABM2, and ABM3 binding sites are present in a 2:0:4 ratio. FIG. 14B shows an example of a MIAC with binding sites present in this ratio.

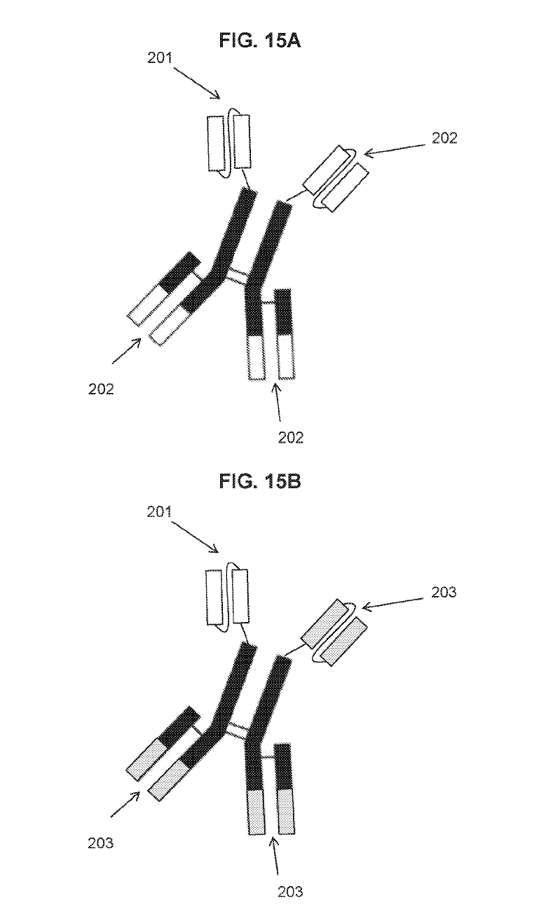

In some embodiments, ABM1, ABM2, and ABM3 binding sites are present in a 1:3:0 ratio. FIG. 15A shows an example of a MIAC with binding sites present in this ratio.

In some embodiments, ABM1, ABM2, and ABM3 binding sites are present in a 1:0:3 ratio. FIG. 15B shows an example of a MIAC with binding sites present in this ratio.

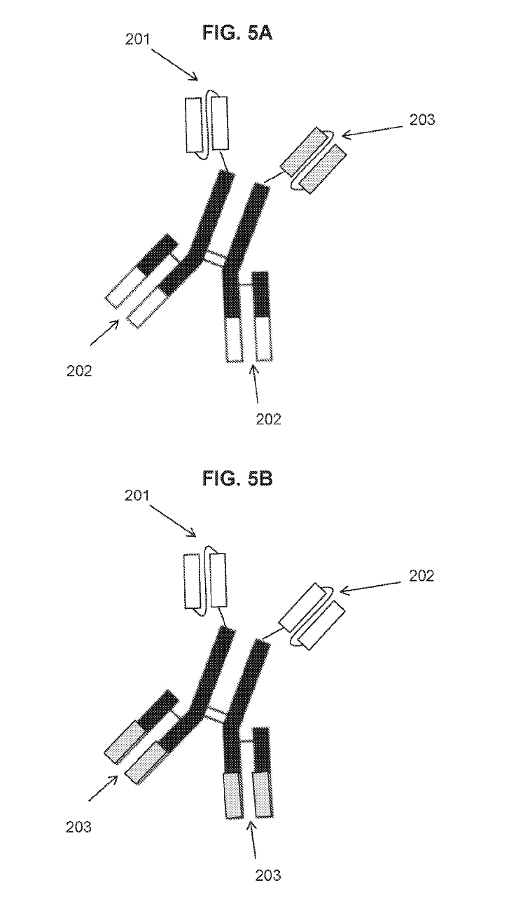

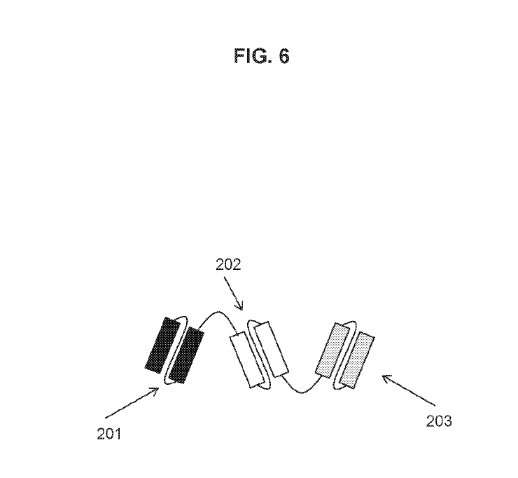

Other suitable ratios for ABM1, ABM2, and ABM3 binding sites include 1:1:1 (e.g., FIG. 6), 1:2:1 (e.g., FIG. 5A), 1:1:2 (e.g., FIG. 5B), 2:1:2, 2:2:1, and the like. A skilled person will readily recognize that the ratio of binding sites contributed by the different modules is not limiting, and that the proper ratio can be selected based on the intended biological activity of the MIAC.

In some embodiments, provided herein are MIACs with ABM1, ABM2, and ABM3 binding sites each present in a ratio of 1-10:0-10:0-10. In particular, provided herein are MIACs with ABM1, ABM2, and ABM3 binding sites each present in a ratio of 1-5:0-5:0-5. For example, in some embodiments, ABM1, ABM2, and ABM3 are present in ratios of 1:0:1, 1:0:2, 1:0:3, 1:0:4, 1:0:5, 1:1:0, 1:1:1, 1:1:2, 1:1:3, 1:1:4, 1:1:5, 1:2:0, 1:2:1, 1:2:2, 1:2:3, 1:2:4, 1:2:5, 1:3:0, 1:3:1, 1:3:2, 1:3:3, 1:3:4, 1:3:5, 1:4:0, 1:4:1, 1:4:2, 1:4:3, 1:4:4, 1:4:5, 1:5:0, 1:5:1, 1:5:2, 1:5:3, 1:5:4, 1:5:5, 2:0:1, 2:0:2, 2:0:3, 2:0:4, 2:0:5, 2:1:0, 2:1:1, 2:1:2, 2:1:3, 2:1:4, 2:1:5, 2:2:0, 2:2:1, 2:2:2, 2:2:3, 2:2:4, 2:2:5, 2:3:0, 2:3:1, 2:3:2, 2:3:3, 2:3:4, 2:3:5, 2:4:0, 2:4:1, 2:4:2, 2:4:3, 2:4:4, 2:4:5, 2:5:0, 2:5:1, 2:5:2, 2:5:3, 2:5:4, 2:5:5, 3:0:1, 3:0:2, 3:0:3, 3:0:4, 3:0:5, 3:1:0, 3:1:1, 3:1:2, 3:1:3, 3:1:4, 3:1:5, 3:2:0, 3:2:1, 3:2:2, 3:2:3, 3:2:4, 3:2:5, 3:3:0, 3:3:1, 3:3:2, 3:3:3, 3:3:4, 3:3:5, 3:4:0, 3:4:1, 3:4:2, 3:4:3, 3:4:4, 3:4:5, 3:5:0, 3:5:1, 3:5:2, 3:5:3, 3:5:4, 3:5:5, 4:0:1, 4:0:2, 4:0:3, 4:0:4, 4:0:5, 4:1:0, 4:1:1, 4:1:2, 4:1:3, 4:1:4, 4:1:5, 4:2:0, 4:2:1, 4:2:2, 4:2:3, 4:2:4, 4:2:5, 4:3:0, 4:3:1, 4:3:2, 4:3:3, 4:3:4, 4:3:5, 4:4:0, 4:4:1, 4:4:2, 4:4:3, 4:4:4, 4:4:5, 4:5:0, 4:5:1, 4:5:2, 4:5:3, 4:5:4, 4:5:5, 4:0:1, 5:0:2, 5:0:3, 5:0:4, 5:0:5, 5:1:0, 5:1:1, 5:1:2, 5:1:3, 5:1:4, 5:1:5, 5:2:0, 5:2:1, 5:2:2, 5:2:3, 5:2:4, 5:2:5, 5:3:0, 5:3:1, 5:3:2, 5:3:3, 5:3:4, 5:3:5, 5:4:0, 5:4:1, 5:4:2, 5:4:3, 5:4:4, 5:4:5, 5:5:0, 5:5:1, 5:5:2, 5:5:3, 5:5:4, or 5:5:5.

Similarly, the affinities of each module can also be adjusted to tailor the targeting (ABM1), agonistic (ABM2), and antagonist (ABM3) effects of the MIACs.

In particular, the ratio of the affinities between ABM2 and ABM3 will affect the degree of activation of the effector cell. Suitable ABM2:ABM3 affinity ratios can range, for example, from 1:100 to 100:1. In some embodiments, the ABM2: ABM3 affinity ratio is about 1:100, 1:90, 1:80, 1:70, 1:60, 1:50, 1:40, 1:30, 1:20, 1:10, 1:1, 10:1, 20:1, 30:1, 40:1, 50:1, 60:1, 70:1, 80:1, 90:1, or 100:1. In some embodiments, the ABM2: ABM3 affinity ratio is at least 1:100, 1:90, 1:80, 1:70, 1:60, 1:50, 1:40, 1:30, 1:20, 1:10, 1:1, 10:1, 20:1, 30:1, 40:1, 50:1, 60:1, 70:1, 80:1, 90:1, or 100:1. In some embodiments, the ABM2: ABM3 affinity ratio is at most 1:100, 1:90, 1:80, 1:70, 1:60, 1:50, 1:40, 1:30, 1:20, 1:10, 1:1, 10:1, 20:1, 30:1, 40:1, 50:1, 60:1, 70:1, 80:1, 90:1, or 100:1.

The MIACs provided herein can comprise any suitable number of any of the ABMs provided herein. In some embodiments, a MIAC provided herein comprises 1, 2, 3, 4, 5, 6, 7, 8, 9, or 10 ABM's. In some embodiments, a MIAC provided herein comprises 0, 1, 2, 3, 4, 5, 6, 7, 8, 9, or 10 ABM2s. In some embodiments, a MIAC provided herein comprises 0, 1, 2, 3, 4, 5, 6, 7, 8, 9, or 10 ABM3s. In some embodiments, a MIAC provided herein comprises 0, 1, 2, 3, 4, 5, 6, 7, 8, 9, or 10 ABM4s.

The MIACs provided herein can also comprise ABMs with any suitable number of binding sites per ABM. In some embodiments, an ABM1 used in a MIAC provided herein comprises 1, 2, 3, 4, 5, 6, 7, 8, 9, or 10 binding sites for a cancer cell antigen. In some embodiments, an ABM2 used in a MIAC provided herein comprises 1, 2, 3, 4, 5, 6, 7, 8, 9, or 10 binding sites for an activating receptor on an effector cell. In some embodiments, an ABM3 used in a MIAC provided herein comprises 1, 2, 3, 4, 5, 6, 7, 8, 9, or 10 binding sites for an inhibitory receptor on an effector cell. In some embodiments, an ABM4 used in a MIAC provided herein comprises 1, 2, 3, 4, 5, 6, 7, 8, 9, or 10 binding sites for an Fc receptor on an effector cell.

Moreover, the MIACs provided herein can also comprise ABMs that target different cancer cell antigens, different activating receptors, and/or different inhibitory receptors.

For example, in some embodiments, a MIAC provided herein comprises an ABM1 that targets more than one cancer cell antigen. In some embodiments, the ABM1 targets 2, 3, 4, 5, 6, 7, 8, 9, 10, or more different cancer cell antigens.

In some embodiments, a MIAC provided herein comprises more than one ABM1, wherein each ABM1 targets a different cancer cell antigen. In some embodiments, the different ABM's collectively target 2, 3, 4, 5, 6, 7, 8, 9, 10, or more different cancer cell antigens. In some embodiments, some ABM's target the same cancer cell antigen(s) and some ABM's target different cancer cell antigens, but the ABM's collectively target 2, 3, 4, 5, 6, 7, 8, 9, 10, or more different cancer cell antigens.

In some embodiments, a MIAC provided herein comprises an ABM2 that targets more than one activating receptor. In some embodiments, the ABM2 targets 2, 3, 4, 5, 6, 7, 8, 9, 10, or more different activating receptors.

In some embodiments, a MIAC provided herein comprises more than one ABM2, wherein each ABM2 targets a different activating receptor. In some embodiments, the different ABM2s collectively target 2, 3, 4, 5, 6, 7, 8, 9, 10, or more different activating receptors. In some embodiments, some ABM2s target the same activating receptor(s) and some ABM2s target different activating receptors, but the ABM2s collectively target 2, 3, 4, 5, 6, 7, 8, 9, 10, or more different activating receptors.

In some embodiments, a MIAC provided herein comprises an ABM3 that targets more than one inhibitory receptor. In some embodiments, the ABM3 targets 2, 3, 4, 5, 6, 7, 8, 9, 10, or more different inhibitory receptors.

In some embodiments, a MIAC provided herein comprises more than one ABM3, wherein each ABM3 targets a different inhibitory receptor. In some embodiments, the different ABM3s collectively target 2, 3, 4, 5, 6, 7, 8, 9, 10, or more different inhibitory receptors. In some embodiments, some ABM3s target the same inhibitory receptor(s) and some ABM3s target different inhibitory receptors, but the ABM3s collectively target 2, 3, 4, 5, 6, 7, 8, 9, 10, or more different inhibitory receptors.

3.2. Antigen-Binding Module 1 (ABM1): Cancer Cell Antigen Binder

In the MIACs provided herein, ABM1 binds specifically to an antigen expressed by a cancer cell (a “cancer cell antigen”). Preferred antigens bound by ABM1 include those that are expressed by cancer cells but not normal cells, or those that are upregulated in cancer cells in comparison to normal cells. It is preferred that the antigen is expressed on the surface of the cell, where it is accessible to ABM1. Any suitable cancer cell antigen can be targeted by ABM1, and a skilled person will be capable of selecting a suitable antigen for binding by ABM.

In some embodiments, an antigen bound by ABM1 is upregulated in the cancer cell by at least a certain amount in comparison to a control cell. The control cell can be a cell from the same tissue. The cell from the same tissue can be a normal version of a type of cell that can develop into the cancer cell to be targeted. For example, if the cancer cell is a human colon cancer epithelial cell, then the control cell can be a normal human colon epithelial cell. This example is provided for illustrative purposes, and one of ordinary skill in the art can readily select an appropriate control cell to compare to a cancer cell.

In some embodiments, an antigen bound by ABM1 is upregulated by at least 2-fold, in comparison to the control cell. In some embodiments, an antigen bound by ABM1 is upregulated by at least 5-fold, in comparison to the control cell. In some embodiments, an antigen bound by ABM1 is upregulated by at least 10-fold, in comparison to the control cell. In some embodiments, an antigen bound by ABM1 is upregulated by at least 100-fold, in comparison to the control cell. In some embodiments, an antigen bound by ABM1 is upregulated by at least 1,000-fold, in comparison to the control cell. In some embodiments, an antigen bound by ABM1 is upregulated by at least 10,000-fold, in comparison to the control cell. In some embodiments, an antigen bound by ABM1 is upregulated by at least 100,000-fold, in comparison to the control cell. In some embodiments, an antigen bound by ABM1 is upregulated by at least 1,000,000-fold, in comparison to the control cell.

The role of ABM1 is to target the MIAC to a site of malignancy, for example, to a cancer cell. It is therefore not required that ABM1 exert any particular biological activity (i.e., agonizing or antagonizing activity) after binding to its antigen. However, MIACs where ABM1 exerts biological activity by binding to its antigen are also within the scope of the invention. In some embodiments, ABM1 agonizes a receptor antigen. In some embodiments, ABM1 antagonizes a receptor antigen. In some embodiments, ABM1 binds the antigen (including a receptor antigen) without an agonistic or antagonistic effect.

Illustrative cancer cell antigens for binding by ABM1 include, for example, 9-O-acetyl-GD3, βhCG, A33 antigen, CA19-9 marker, CA-125 marker, calreticulin, carboanhydrase IX (MN/CA IX), CCR5, CCR8, CD19, CD20, CD22 (SIGLEC-2), CD25, CD27 (TNFRSF7), CD30 (TNFRSF8), CD33 (SIGLEC-3), CD38 (cyclic ADP ribose hydrolase), CD44v6, CD63 (LAMP-3), CD66e (CEACAM5), CD70, CD123 (IL3RA), CD138 (syndecan 1), CD248 (endosialin) carcinoma embryonic antigen (CEA), desmoglein 4, E-cadherin neoepitope, ephrin A2 (EphA2), epidermal growth factor receptor (EGFR), epithelial cell adhesion molecule (EpCAM), ErbB2, fetal acetylcholine receptor, fibroblast activation antigen (FAP), fucosyl GM1, GD2, GD3, GM2, ganglioside GD3, Globo H, glycoprotein 100 (gp100), HER2/neu, HER3, HER4, insulin-like growth factor receptor 1, Lewis-Y, LG, Ly-6, melanoma-specific chondroitin-sulfate proteoglycan (MCSCP), mesothelin, MUC1, MUC2, MUC3, MUC4, MUC5_{AC}, MUC5_{B}, MUC7, MUC16, Müllerian inhibitory substance (MIS) receptor type II, plasma cell antigen, poly SA, PSCA, PSMA, sonic hedgehog (SHH), SAS, STEAP, sTn antigen, and TNF-alpha precursor. Examples of cancer antigens that can be targeted by ABM1 are provided in U.S. Pat. No. 7,235,641, which is incorporated by reference in their entirety.

In some embodiments, ABM1 is a multispecific antigen-binding module. Multispecific antigen binding modules can be prepared by any methods known in the art, or described herein, such as the knobs and holes approach or combining single domain antibodies known to bind different antigens. In some embodiments a multispecific ABM1 binds 2, 3, 4, 5, 6, 7, 8, or more different antigens. In some embodiments, each of the different antigens is a different antigen recognized by an ABM1 binding site.

In some embodiments, hybrid multispecific ABMs can be formed that comprise more than one type of ABM binding site. For example, in some embodiments, a hybrid multispecific ABM comprises a binding site for ABM1 and ABM2. One example of such a hybrid ABM is a bispecific IgG where one binding site forms a binding site for ABM1 and one binding site forms a binding site for ABM2. Also provided herein are multispecific hybrid ABMs binding ABM1 and 3; ABM1 and 4; ABM1, 2, and 3; ABM1, 2, and 4; ABM1, 3, and 4; and ABM1, 2, 3, and 4.

3.3. Antigen-Binding Module 2 (ABM2): Agonist of Activating Receptor

In the MIACs provided herein, ABM2 binds specifically to an activating receptor expressed by an effector cell. The binding of ABM2 to the activating receptor agonizes the activating receptor, resulting in the transduction of an activating signal to the effector cell. The activity of the effector cell can be tuned, for example, by varying the affinity of ABM2 for the activating receptor, the valency of ABM2, or the number of ABM2s, thereby varying the strength of the activating signal.

The activating receptor targeted by ABM2 is selected, for example, based on the type of effector cell that one wishes to recruit to the cancer cell. For example, in one illustrative embodiment, a natural killer (NK) cell can be recruited and activated by utilizing a CD137-binding molecule with agonizing activity as ABM2.

In some embodiments, the effector cell is an NK cell. Suitable illustrative NK cell receptors for agonism by ABM2 include, for example, 2B4 (CD244), a₄β₁ integrin, β₂ integrins (e.g., CD11α-CD18, CD11b-CD18, CD11c-CD18), CD2 (LFA2, OX34), CD16, CD27 (TNFRSF7), CD38, CD96, CD100, CD160, CD137, CEACAM1 (CD66), CRTAM, CS1 (CD319), DNAM-1 (CD226), GITR (TNFRSF18), activating forms of KIR (e.g., KIR2DS1, KIR2DS4, KIR-S), NKG2C, NKG2D, NKG2E, the natural cytotoxicity receptors (e.g., NKp30, NKp44, NKp46, NKp80), NTB-A, and PEN-5. More information on suitable NK cell receptors for agonism by ABM2 is provided in Miller, Hematology, 2013, 2013(1):247-253; Mentlik et al., Frontiers in Immunology, 2013, 4:481(1-12); Stein et al., Antibodies, 2012, 1:88-123; Pegram et al., Immunology and Cell Biology, 2011, 89:216-224; and Vivier et al., Nature Immunology, 2008, 9:503-510; each of which is incorporated by reference in its entirety.

In some embodiments, the effector cell is a T lymphocyte. In some embodiments, the T lymphocyte is a cytotoxic T lymphocyte. In some embodiments, the T lymphocyte is a γδ T cell. In some embodiments, the T lymphocyte is an NKT cell. In some embodiments, the NKT cell is an iNKT cell. Suitable illustrative T lymphocyte receptors for agonism by ABM2 include, for example, CD2 (LFA2, OX34), CD3, CD5, CD27 (TNFRSF7), CD28, CD30 (TNFRSF8), CD40L, CD84 (SLAMF5), CD137 (4-1BB), CD226, CD229 (Ly9, SLAMF3), CD244 (2B4, SLAMF4), CD319 (CRACC, BLAME), CD352 (Ly108, NTBA, SLAMF6), CRTAM (CD355), DR3 (TNFRSF25), GITR (CD357), HVEM (CD270), ICOS, LIGHT, LTβR (TNFRSF3), OX40 (CD134), NKG2D, SLAM (CD150, SLAMF1), TCRα, TCRβ, TCRδγ, and TIM1 (HAVCR, KIM1). More information on suitable T cell receptors for agonism by ABM2 is provided in Stein et al., Antibodies, 2012, 1:88-123; Chen and Flies, Nature Reviews Immunology, 2013, 13:227-242; and Pardoll, Nature Reviews Cancer, 2012, 12:252-264; each of which is incorporated by reference in its entirety.

In some embodiments, ABM2 is specifically not a natural ligand for an activating receptor expressed by an effector cell, or a portion thereof. In some embodiments, ABM2 is a natural ligand for an activating receptor expressed by an effector cell, or a portion thereof.

In some embodiments, ABM2 is a multispecific antigen-binding module. Multispecific antigen binding modules can be prepared by any methods known in the art, or described herein, such as the knobs and holes approach or combining single domain antibodies known to bind different antigens. In some embodiments a multispecific ABM2 binds 2, 3, 4, 5, 6, 7, 8, or more different antigens. In some embodiments, each of the different antigens is a different antigen recognized by an ABM2 binding site.

In some embodiments, hybrid multispecific ABMs can be formed that comprise more than one type of ABM binding site. For example, in some embodiments, a hybrid multispecific ABM comprises a binding site for ABM2 and ABM1. One example of such a hybrid ABM is a bispecific IgG where one binding site forms a binding site for ABM2 and one binding site forms a binding site for ABM1. Also provided herein are multispecific hybrid ABMs binding ABM2 and 3; ABM2 and 4; ABM2, 1, and 3; ABM2, 1, and 4; ABM2, 3, and 4; and ABM2, 1, 3, and 4.

3.4. Antigen-Binding Module 3 (ABM3): Antagonist of Inhibitory Receptor

In the MIACs provided herein, ABM3 binds specifically to an inhibitory receptor expressed by an effector cell. The binding of ABM3 to the inhibitory receptor antagonizes the inhibitory receptor, resulting in the blockage of inhibitory signals that are transduced to the effector cell. The activity of the effector cell can be further tuned, for example, by varying the affinity of ABM3 for the inhibitory receptor, the valency of ABM3, or the number of ABM3s, thereby varying the degree of antagonism of the inhibitory signal.

The inhibitory receptor targeted by ABM3 is selected, for example, based on the type of effector cell that one wishes to recruit to the cancer cell. For example, in the illustrative embodiment where the effector cell is an NK cell, ABM3 can be a KIR2DL1-binding molecule with antagonizing activity.

In some embodiments, the effector cell is an NK cell. Suitable illustrative NK cell receptors for antagonism by ABM3 include, for example, ILT2/LIR-1/CD85j, inhibitory forms of KIR (e.g., KIR2DL1, KIR2DL2, KIR2DL3, KIR3DL1, KIR3DL2, KIR-L), KLRG1, LAIR-1, NKG2A, NKR-P1A, Siglec-3, Siglec-7, and Siglec-9. More information on NK cell receptors for antagonism by ABM3 is provided in Miller, Hematology, 2013, 2013(1):247-253; Mentlik et al., Frontiers in Immunology, 2013, 4:481(1-12); Stein et al., Antibodies, 2012, 1:88-123; Pegram et al., Immunology and Cell Biology, 2011, 89:216-224; and Vivier et al., Nature Immunology, 2008, 9:503-510; each of which is incorporated by reference in its entirety.

In some embodiments, the effector cell is a T lymphocyte. In some embodiments, the T lymphocyte is a cytotoxic T lymphocyte. In some embodiments, the T lymphocyte is a γδ T cell. In some embodiments, the T lymphocyte is an NKT cell. In some embodiments, the NKT cell is an iNKT cell. Suitable illustrative T lymphocyte receptors for antagonism by ABM3 include, for example, 2B4 (CD244, SLAMF4), B71 (CD80), B7H1 (CD274, PD-L1), BTLA (CD272), CD160 (BY55, NK28), CD352 (Ly108, NTBA, SLAMF6), CD358 (DR6), CTLA-4 (CD152), LAG3, LAIR1, PD-1 (CD279), PD-1H (VISTA), TIGIT (VSIG9, VSTM3), TIM2 (TIMD2), and TIM3 (HAVCR2, KIM3). More information on T cell receptors for antagonism by ABM3 is provided in Stein et al., Antibodies, 2012, 1:88-123; Chen and Flies, Nature Reviews Immunology, 2013, 13:227-242; and Pardoll, Nature Reviews Cancer, 2012, 12:252-264; each of which is incorporated by reference in its entirety.

In some embodiments, ABM3 is specifically not a natural ligand for an inhibitory receptor expressed by an effector cell, or a portion thereof. In some embodiments, ABM3 is a natural ligand for an inhibitory receptor expressed by an effector cell, or a portion thereof.

In some embodiments, ABM3 is a multispecific antigen-binding module. Multispecific antigen binding modules can be prepared by any methods known in the art, or described herein, such as the knobs and holes approach or combining single domain antibodies known to bind different antigens. In some embodiments a multispecific ABM3 binds 2, 3, 4, 5, 6, 7, 8, or more different antigens. In some embodiments, each of the different antigens is a different antigen recognized by an ABM3 binding site.

In some embodiments, hybrid multispecific ABMs can be formed that comprise more than one type of ABM binding site. For example, in some embodiments, a hybrid multispecific ABM comprises a binding site for ABM3 and ABM1. One example of such a hybrid ABM is a bispecific IgG where one binding site forms a binding site for ABM3 and one binding site forms a binding site for ABM1. Also provided herein are multispecific hybrid ABMs binding ABM3 and 2; ABM3 and 4; ABM3, 1, and 2; ABM3, 1, and 4; ABM3, 2, and 4; and ABM3, 1, 2, and 4.

3.5. Illustrative Examples of ABM1, ABM2, and ABM3 Combinations

Any suitable ABM1, ABM2, and/or ABM3 can be combined to produce a MIAC herein. The following combinations are provided solely for illustrative purposes and are not intended to limit the invention to any particular combination of ABM1, ABM2, and ABM3.

In some embodiments, the MIAC comprises an ABM1, ABM2, and ABM3, wherein ABM1 binds CD30, ABM2 agonizes CD137, and ABM3 antagonizes PD-1. In some embodiments the MIAC comprises an ABM1 and ABM2, wherein ABM1 binds CD30 and ABM2 agonizes CD137. In some embodiments the MIAC comprises an ABM1 and ABM3, wherein ABM1 binds CD30 and ABM3 antagonizes PD-1. In some embodiments, any of the constructs described in this paragraph comprise an ABM4 binding CD64.

In some embodiments, the MIAC comprises an ABM1, ABM2, and ABM3, wherein ABM1 binds CD30, ABM2 agonizes NKG2D, and ABM3 antagonizes an inhibitory form of KIR. In some embodiments the MIAC comprises an ABM1 and ABM2, wherein ABM1 binds CD30 and ABM2 agonizes NKG2D. In some embodiments the MIAC comprises an ABM1 and ABM3, wherein ABM1 binds CD30 and ABM3 antagonizes an inhibitory form of KIR. In some embodiments, any of the constructs described in this paragraph comprise an ABM4 binding CD64.

In some embodiments, the MIAC comprises an ABM1, ABM2, and ABM3, wherein ABM1 binds CD30, ABM2 agonizes CD137, and ABM3 antagonizes an inhibitory form of KIR. In some embodiments the MIAC comprises an ABM1 and ABM2, wherein ABM1 binds CD30 and ABM2 agonizes CD137. In some embodiments the MIAC comprises an ABM1 and ABM3, wherein ABM1 binds CD30 and ABM3 antagonizes an inhibitory form of KIR. In some embodiments, any of the constructs described in this paragraph comprise an ABM4 binding CD64.

In some embodiments, the MIAC comprises an ABM1, ABM2, and ABM3, wherein ABM1 binds CD20, ABM2 agonizes NKG2D, and ABM3 antagonizes an inhibitory form of KIR. In some embodiments the MIAC comprises an ABM1 and ABM2, wherein ABM1 binds CD20 and ABM2 agonizes NKG2D. In some embodiments the MIAC comprises an ABM1 and ABM3, wherein ABM1 binds CD20 and ABM3 antagonizes an inhibitory form of KIR. In some embodiments, any of the constructs described in this paragraph comprise an ABM4 binding CD64.

In some embodiments, the MIAC comprises an ABM1, ABM2, and ABM3, wherein ABM1 binds CD30, ABM2 agonizes NKG2D, and ABM3 antagonizes NKG2A. In some embodiments the MIAC comprises an ABM1 and ABM2, wherein ABM1 binds CD30 and ABM2 agonizes NKG2D. In some embodiments the MIAC comprises an ABM1 and ABM3, wherein ABM1 binds CD30 and ABM3 antagonizes NKG2A. In some embodiments, any of the constructs described in this paragraph comprise an ABM4 binding CD64.

3.6. Antigen-Binding Module 4 (ABM4): Fc Receptor-Binding Module

In some embodiments, the MIACs provided herein comprise a binding module that binds to an Fc receptor on an effector cell. Fc receptors include, for example, CD16 (CD16a, CD16b), CD32a, CD64, and CD89.

In some embodiments, ABM4 is an immunoglobulin, antibody, antibody fragment, or alternative scaffold that specifically binds an Fc receptor. In some embodiments, ABM4 is an Fc domain of an immunoglobulin.

Although an Fc domain of an immunoglobulin is not generally described in the art as “antigen-binding,” for purposes of ABM4 of the MIACs of this disclosure, the receptors for the Fc domain are considered “antigens” bound by the Fc domain. In other words, the Fc domain of an immunoglobulin is one type of ABM4, among the other types of ABM4s described in the preceding paragraph and throughout this disclosure.

For example, where ABM1, ABM2, and/or ABM3 comprise an immunoglobulin protein with an Fc domain, then an Fc domain can be present. More particularly, and by way of illustration, where any of ABM1, ABM2, or ABM3 comprises an IgG, then ABM4 can be an Fc domain of the IgG. In these embodiments, a single IgG can form ABM4 and at least one of ABM1, ABM2, and ABM3, as will be readily recognized by one of skill in the art. FcR expression on effector cells is summarized in Ravetch and Kinet, Ann. Rev. Immunol., 1991, 9:457-492, which is incorporated by reference in its entirety.

In certain embodiments, modifications can be introduced into the Fc region to generate an Fc region variant. In certain embodiments, the Fc region variant possesses an enhanced or otherwise altered effector function. Numerous substitutions or substitutions or deletions with altered effector function are known in the art. Examples of Fc region variants include those described in U.S. Pat. No. 8,815,237; Lazar et al., Proc. Natl. Acad. Sci. USA, 2006, 103:4005-4010; and Strohl, Current Opinion in Biotechnology, 2009, 20:685-691; each of which is incorporated by reference in its entirety.

An alteration in complement-dependent cytotoxicity (CDC) and/or antibody-dependent cell-mediated cytotoxicity (ADCC) activity can be confirmed using in vitro and/or in vivo assays. For example, Fc receptor binding assays can be conducted to measure FcγR binding. Non-limiting examples of in vitro assays to assess ADCC activity of a molecule of interest are provided in U.S. Pat. Nos. 5,500,362 and 5,821,337; Hellstrom et al., Proc. Natl. Acad. Sci. USA., 1986, 83:7059-7063; Hellstrom et al., Proc. Natl. Acad. Sci. USA., 1985, 82:1499-1502; and Bruggemann et al., J. Exp. Med., 1987, 166:1351-1361, each of which is incorporated by reference in its entirety. Alternatively, or additionally, ADCC activity of a molecule of interest can be assessed in vivo, using an animal model such as that disclosed in Clynes et al. Proc. Natl. Acad. Sci. USA., 1998, 95:652-656, which is incorporated by reference in its entirety.

In some embodiments, the MIACs do not comprise an ABM4.

In the context of ABM4, Fc can be modified, e.g., to include one or more modifications in a CH2 domain and/or a CH3 domain. Such modifications can impact Fc function and binding characteristics such as Fc receptor (FcR) binding. Fc can be modified to block binding to FcR, for example Fc can be modified to include a mutation to block FcR binding such as a mutation at amino acid N297. Fc can be modified to prevent N-linked glycosylation and/or reduce ADCC.

3.7. Assembly of the Antigen-Binding Modules

The MIACs provided herein are characterized in that the ABMs that comprise the MIAC are associated (or “bound”), covalently or non-covalently, with each other to form the MIAC. A skilled person is capable of selecting the type of association based on the nature of the ABMs and the application.

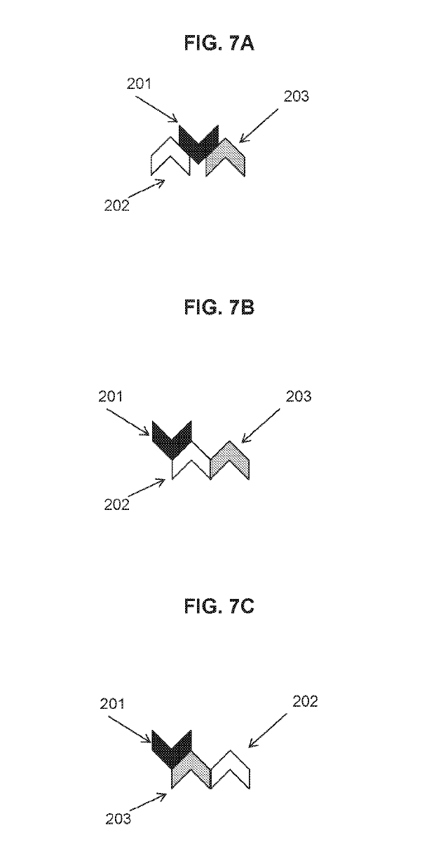

In some embodiments, one ABM is bound to both of the other two ABMs, but the other two ABMs are not bound directly to each other. For example, as shown in FIG. 7A, in some aspects ABM1 is bound to both ABM2 and ABM3, while ABM2 and ABM3 are not bound to each other. In some aspects, as shown in FIG. 7B, ABM2 is bound to both ABM1 and ABM3, while ABM1 and ABM3 are not bound directly to each other. In some aspects, as shown in FIG. 7C, ABM3 is bound to both ABM1 and ABM2, while ABM1 and ABM2 are not bound directly to each other.

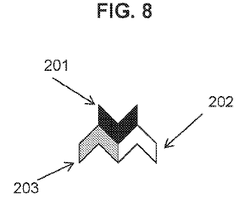

In some embodiments, each ABM is bound to two other ABMs. For example, in some aspects, as shown in FIG. 8, ABM1 is bound to ABM2 and ABM3; ABM2 is bound to ABM1 and ABM3; and ABM3 is bound to ABM1 and ABM2.

In embodiments where MIACs comprise only two ABMs, the ABMs will generally be covalently or non-covalently associated with each other. However, in some embodiments, each ABM can be associated with a third molecule that is not an ABM.

3.7.1. Covalently-Associated ABMs

In some embodiments, the ABMs are covalently associated with each other. The covalent association can be any suitable covalent linkage.

In some embodiments, the covalent association is in the form of a fusion protein comprising two or more ABMs, or portions thereof. Illustrative embodiments of such fusion proteins include fusion proteins comprising an scFv and the heavy- or light-chain of an IgG, as illustrated in FIGS. 2A-5B and 11A-15B. A further illustrative embodiment of a fusion protein is the MIAC depicted in FIGS. 6 and 16A-18B.

The MIACs provided herein can comprise any suitable fusion protein structure, and the selection of the appropriate fusion protein can be carried out by one of skill in the art depending, for example, upon the desired valency and molecular weight of each ABM of the MIAC. Examples of suitable fusion protein structures are provided throughout this disclosure. Methods of producing fusion proteins are described elsewhere in this disclosure.

In some embodiments, the fusion proteins comprise a polypeptide linker. The polypeptide linker can be any suitable polypeptide linker that attaches at least two proteins (e.g., ABMs) of the fusion protein to each other. A skilled artisan is capable of selecting appropriate polypeptide linkers based on the components of the fusion protein (e.g., the ABMs) and its applications. Examples of suitable linkers include (GGGGS)ₙ, (SEQ ID NO: 20), the Fc interlinker from human IgG1 C_{H2} residues 297-322: NSTYRVVSVLTVLHQDWLNGKEYKCK (SEQ ID NO: 21), and the HAS interlinker from the D3 domain of human serum albumin: FQNALLVRYTKKVPQVSTPTLVEVS (SEQ ID NO: 22). See Fang et al., Chines. Sci. Bull., 2003, 48:1912-1918, incorporated by reference in its entirety. In some embodiments, the linker is (GGGGS)₃, (SEQ ID NO: 23). Other linkers are provided, for example, in U.S. Pat. No. 5,525,491; Alfthan et al., Protein Eng., 1995, 8:725-731; Shan et al., J. Immunol., 1999, 162:6589-6595; Newton et al., Biochemistry, 1996, 35:545-553; Megeed et al.; Biomacromolecules, 2006, 7:999-1004; and Perisic et al., Structure, 1994, 12:1217-1226; each of which is incorporated by reference in its entirety.

In some embodiments, the ABMs are covalently associated by a chemical coupling. Any suitable chemical linker can be used to covalently associate the ABMs provided herein. Chemical coupling of antibodies to each other is described, for example, in Wong et al., Scand. J. Rheumatol., 2000, 29:282-287; Jung et al., Eur. J. Immunol., 1991, 21:2431-2435; Tutt et al., J. Immunol., 1991, 147:60-69; French, Methods Mol. Biol., 1998, 80:121-134; and Gavrilyuk et al., Bioorg. Med. Chem. Lett., 2009, 19:3716-3720; each of which is incorporated by reference in its entirety.

In some embodiments, the chemical coupling is via a spacer. In some embodiments, the spacer is a molecule selected from a polymer, a polypeptide, a carbohydrate (e.g., dextran), or the like. In particular embodiments, the spacer is a poly(ethylene) glycol (PEG) polymer. In some embodiments, the PEG has a molecular weight in the range of about 2.5 kDa to about 50 kDa. PEG reagents for chemical coupling, and methods of their use, are described, for example, in Hermanson, Bioconjugate Techniques, 2013, 3d ed., chapter 18, Academic Press, London, UK, Waltham Mass., and San Diego, Calif., which is incorporated by reference in its entirety.

3.7.2. Non-Covalently Associated ABMs

In some embodiments, the ABMs are non-covalently associated with each other. The non-covalent association can be any suitable covalent linkage.

In some embodiments, the non-covalent association is in the form of a specific interaction between two molecules. For example, in some embodiments, the non-covalent association is an interaction between avidin and biotin. In some embodiments, the avidin is selected from a streptavidin and a neutravidin. In these embodiments, an avidin molecule is attached to one ABM and a biotin molecule is attached to another ABM. The ABMs then associate as a result of the specific, high affinity interaction between the avidin and the biotin. Avidin-biotin systems, and methods of their use, are described, for example, in Hermanson, Bioconjugate Techniques, 2013, 3d ed., chapter 11, Academic Press, London, UK, Waltham Mass., and San Diego, Calif., which is incorporated by reference in its entirety.

3.8. Illustrative Embodiments of Multispecific Immunomodulatory Constructs (MIACs)

FIGS. 2A-6 and 11A-18B provide illustrative, non-limiting, examples of the MIACs provided herein.

In FIG. 2A, ABM1 (201) is an IgG, ABM2 (202) is an scFv, and ABM3 (203) is an scFv. ABM2 (202) and ABM3 (203) are attached to the C-termini of the heavy chains of the IgG, using a polypeptide linker. However, it is also possible to attach one or both of ABM2 (202) and ABM3 (203) to the N-termini of the heavy chains of the IgG.

In FIG. 2B, ABM1 (201) is an IgG, ABM2 (202) is an scFv, and ABM3 (203) is an scFv. ABM2 (202) and ABM3 (203) are attached to the C-termini of the light chains of the IgG, using a polypeptide linker. However, it is also possible to attach one or both of ABM2 (202) and ABM3 (203) to the N-termini of the light chains of the IgG.

In FIG. 2C, ABM1 (201) is an IgG, ABM2 (202) is an scFv, and ABM3 (203) is an scFv. ABM2 (202) is attached to the C-terminus of one light chain of the IgG, using a polypeptide linker. ABM3 (203) is attached to the C-terminus of one heavy chain of the IgG, using a polypeptide linker. However, it is also possible to attach one or both of ABM2 (202) and ABM3 (203) to the N-terminus of the light chain, or the N-terminus of the heavy chain, respectively.

In FIG. 2D, ABM1 (201) is an IgG, ABM2 (202) is an scFv, and ABM3 (203) is an scFv. ABM2 (202) is attached to the C-terminus of one heavy chain of the IgG, using a polypeptide linker. ABM3 (203) is attached to the C-terminus of one light chain of the IgG, using a polypeptide linker. However, it is also possible to attach one or both of ABM2 (202) and ABM3 (203) to the N-terminus of the heavy chain, or the N-terminus of the light chain, respectively.

In FIG. 3A, ABM1 (201) is an IgG, ABM2 (202) is an scFv, and ABM3 (203) is an scFv. ABM2 (202) is attached to the C-terminus of one heavy chain of the IgG, using a polypeptide linker. ABM3 (203) is attached to the C-terminus of the other heavy chain of the IgG, and one light chain of the IgG, using a polypeptide linker. However, it is also possible to attach one or both of ABM2 (202) and ABM3 (203) to the N-termini of the heavy chains, or the N-terminus of the light chain.

In FIG. 3B, ABM1 (201) is an IgG, ABM2 (202) is an scFv, and ABM3 (203) is an scFv. ABM2 (202) is attached to the C-terminus of one light chain of the IgG, using a polypeptide linker. ABM3 (203) is attached to the C-terminus of the other light chain of the IgG, and one heavy chain of the IgG, using a polypeptide linker. However, it is also possible to attach one or both of ABM2 (202) and ABM3 (203) to the N-termini of the light chains, or the N-terminus of the heavy chain.

In FIG. 3C, ABM1 (201) is an IgG, ABM2 (202) is an scFv, and ABM3 (203) is an scFv. ABM2 (202) is attached to the C-terminus of one heavy chain of the IgG, and one light chain of the IgG, using a polypeptide linker. ABM3 (203) is attached to the C-terminus of the other heavy chain of the IgG, using a polypeptide linker. However, it is also possible to attach one or both of ABM2 (202) and ABM3 (203) to the N-termini of the heavy chains, or the N-terminus of the light chain.

In FIG. 3D, ABM1 (201) is an IgG, ABM2 (202) is an scFv, and ABM3 (203) is an scFv. ABM2 (202) is attached to the C-terminus of one heavy chain of the IgG, and one light chain of the IgG, using a polypeptide linker. ABM3 (203) is attached to the C-terminus of the other light chain of the IgG, using a polypeptide linker. However, it is also possible to attach one or both of ABM2 (202) and ABM3 (203) to the N-termini of the light chains, or the N-terminus of the heavy chain.

In FIG. 4A, ABM1 (201) is an IgG, ABM2 (202) is an scFv, and ABM3 (203) is an scFv. ABM2 (202) is attached to the C-terminus of the light chain on one half of the IgG and the C-terminus of the heavy chain on the same half of the IgG. ABM3 (203) is attached to the C-terminus of the light chain on the other half of the IgG and the C-terminus of the heavy chain on the other half of the IgG. However, it is also possible to attach one or more of ABM2 (202) and ABM3 (203) to the N-termini of the light chains, or the N-terminus of the heavy chains.

In FIG. 4B, ABM1 (201) is an IgG, ABM2 (202) is an scFv, and ABM3 (203) is an scFv. ABM2 (202) is attached to the C-terminus of the light chain on one half of the IgG and the C-terminus of the heavy chain on the other half of the IgG. ABM3 (203) is attached to the C-terminus of the light chain on one half of the IgG and the C-terminus of the heavy chain on the other half of the IgG. However, it is also possible to attach one or more of ABM2 (202) and ABM3 (203) to the N-termini of the light chains, or the N-terminus of the heavy chains.

In FIG. 5A, ABM1 (201) is an scFv, ABM2 (202) is an IgG, and ABM3 (203) is an scFv. ABM1 (201) and ABM3 (203) are attached to the C-termini of the heavy chains of the IgG, using a polypeptide linker. However, it is possible to attach one or both of ABM1 (201) and ABM3 (203) to the N-termini of the heavy chains of the IgG. It is also possible to attach ABM1 (201) and ABM3 (203) to any other suitable site of the IgG, including the C- or N-termini of the light chains.

In FIG. 5B, ABM1 (201) is an scFv, ABM2 (202) is an scFv, and ABM3 (203) is an IgG. ABM1 (201) and ABM2 (202) are attached to the C-termini of the heavy chains of the IgG, using a polypeptide linker. However, it is possible to attach one or both of ABM1 (201) and ABM2 (203) to the N-termini of the heavy chains of the IgG. It is also possible to attach ABM1 (201) and ABM2 (203) to any other suitable site of the IgG, including the C- or N-termini of the light chains.

In FIG. 6, ABM1 (201) is an scFv, ABM2 (202) is an scFv, and ABM3 (203) is an scFv. In this illustrative embodiment, the scFvs are assembled, using polypeptide linkers, in the order ABM1-ABM2-ABM3, from N-terminal to C-terminal. However, it is possible to assemble the scFvs in any suitable order, including, for example ABM1-ABM3-ABM2, ABM2-ABM1-ABM3, ABM2-ABM3-ABM1, ABM3-ABM1-ABM2, and ABM3-ABM2-ABM1, from N-terminal to C-terminal.

In FIG. 11A, ABM1 (201) is an IgG and ABM2 (202) is an scFv. One ABM2 (202) is attached to each of the C-termini of the heavy chains of the IgG, using a polypeptide linker. However, it is also possible to attach one or both ABM2s (202) to the N-termini of the heavy chains of the IgG.

In FIG. 11B, ABM1 (201) is an IgG and ABM2 (202) is an scFv. One ABM2 (202) is attached to each of the C-termini of the light chains of the IgG, using a polypeptide linker. However, it is also possible to attach one or both ABM2s (202) to the N-termini of the light chains of the IgG.

In FIG. 11C, ABM1 (201) is an IgG and ABM3 (203) is an scFv. One ABM3 (203) is attached to each of the C-termini of the heavy chains of the IgG, using a polypeptide linker. However, it is also possible to attach one or both ABM3s (203) to the N-termini of the heavy chains of the IgG.

In FIG. 11D, ABM1 (201) is an IgG and ABM3 (203) is an scFv. One ABM3 (203) is attached to each of the C-termini of the light chains of the IgG, using a polypeptide linker. However, it is also possible to attach one or both ABM3s (203) to the N-termini of the light chains of the IgG.

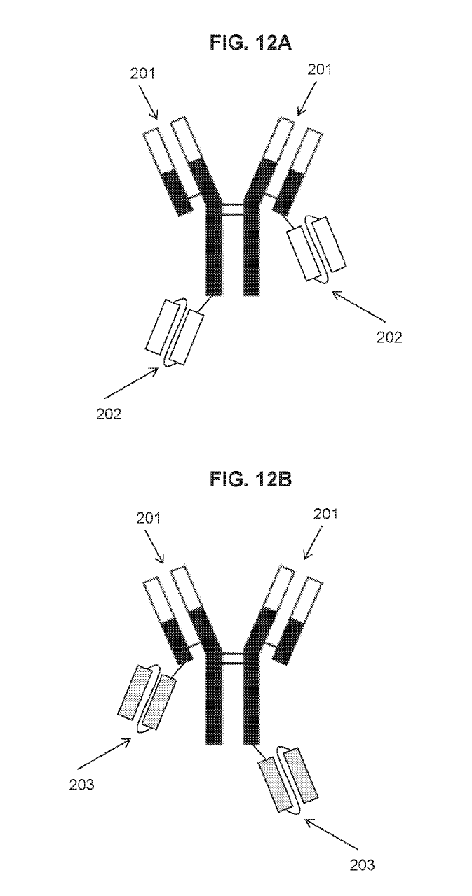

In FIG. 12A, ABM1 (201) is an IgG and ABM2 (202) is an scFv. One ABM2 (202) is attached to the C-terminus of one light chain of the IgG, using a polypeptide linker. Another ABM2 (202) is attached to the C-terminus of one heavy chain of the IgG, using a polypeptide linker. However, it is also possible to attach one or both ABM2s (202) to the N-terminus of the light chain, or the N-terminus of the heavy chain, respectively.

In FIG. 12B, ABM1 (201) is an IgG and ABM3 (203) is an scFv. One ABM3 (203) is attached to the C-terminus of one light chain of the IgG, using a polypeptide linker. Another ABM3 (203) is attached to the C-terminus of one heavy chain of the IgG, using a polypeptide linker. However, it is also possible to attach one or both ABM3s (203) to the N-terminus of the light chain, or the N-terminus of the heavy chain, respectively.

In FIG. 13A, ABM1 (201) is an IgG and ABM2 (202) is an scFv. ABM2 (202) is attached to the C-termini of both heavy chains and one light chain of the IgG, using a polypeptide linker. However, it is also possible to attach any of the ABM2s (202) to the N-termini of the heavy chains, or the N-terminus of the light chain.

In FIG. 13B, ABM1 (201) is an IgG and ABM2 (202) is an scFv. ABM2 (202) is attached to the C-termini of both light chains and one heavy chain of the IgG, using a polypeptide linker. However, it is also possible to attach any of the ABM2s (202) to the N-termini of the light chains, or the N-terminus of the heavy chain.

In FIG. 13C, ABM1 (201) is an IgG and ABM3 (203) is an scFv. ABM3 (203) is attached to the C-termini of both heavy chains and one light chain of the IgG, using a polypeptide linker. However, it is also possible to attach any of the ABM3s (203) to the N-termini of the heavy chains, or the N-terminus of the light chain.

In FIG. 13D, ABM1 (201) is an IgG and ABM3 (203) is an scFv. ABM3 (203) is attached to the C-termini of both light chains and one heavy chain of the IgG, using a polypeptide linker. However, it is also possible to attach any of the ABM3s (203) to the N-termini of the light chains, or the N-terminus of the heavy chain.

In FIG. 14A, ABM1 (201) is an IgG and ABM2 (202) is an scFv. ABM2 (202) is attached to the C-termini of both heavy chains and both light chains. However, it is also possible to attach one or more of the ABM2s (202) to the N-termini of the light chains, or the N-termini of the heavy chains.

In FIG. 14B, ABM1 (201) is an IgG and ABM3 (203) is an scFv. ABM3 (203) is attached to the C-termini of both heavy chains and both light chains. However, it is also possible to attach one or more of the ABM3s (203) to the N-termini of the light chains, or the N-termini of the heavy chains.

In FIG. 15A, ABM1 (201) is an scFv, and ABM2 (202) is an IgG and an scFv. ABM1 (201) and scFv ABM2 (202) are attached to the C-termini of the heavy chains of the IgG, using a polypeptide linker. However, it is possible to attach one or both of ABM1 (201) and scFv ABM2 (202) to the N-termini of the heavy chains of the IgG. It is also possible to attach ABM1 (201) and scFv ABM2 (202) to any other suitable site of the IgG, including the C- or N-termini of the light chains.

In FIG. 15B, ABM1 (201) is an scFv, and ABM3 (203) is an IgG and an scFv. ABM1 (201) and scFv ABM3 (203) are attached to the C-termini of the heavy chains of the IgG, using a polypeptide linker. However, it is possible to attach one or both of ABM1 (201) and scFv ABM3 (203) to the N-termini of the heavy chains of the IgG. It is also possible to attach ABM1 (201) and scFv ABM3 (203) to any other suitable site of the IgG, including the C- or N-termini of the light chains.

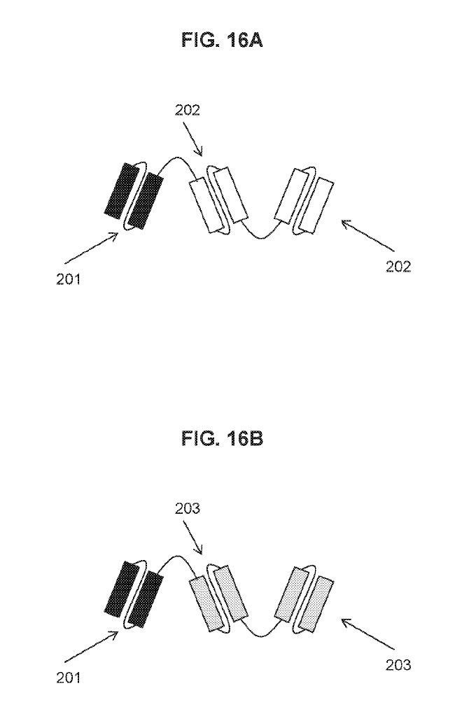

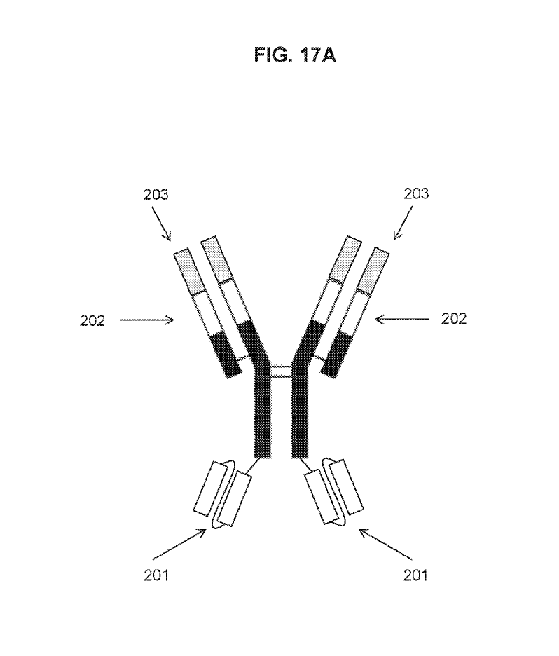

In FIG. 16A, ABM1 (201) is an scFv and both ABM2s (202) are scFvs. In this illustrative embodiment, the scFvs are assembled, using polypeptide linkers, in the order ABM1-ABM2-ABM2, from N-terminal to C-terminal. However, it is possible to assemble the scFvs in any suitable order, including, for example ABM2-ABM1-ABM2, and ABM2-ABM2-ABM1, from N-terminal to C-terminal.

In FIG. 16B, ABM1 (201) is an scFv and both ABM3s (203) are scFvs. In this illustrative embodiment, the scFvs are assembled, using polypeptide linkers, in the order ABM1-ABM3-ABM3, from N-terminal to C-terminal. However, it is possible to assemble the scFvs in any suitable order, including, for example ABM3-ABM1-ABM3, and ABM3-ABM3-ABM1, from N-terminal to C-terminal.

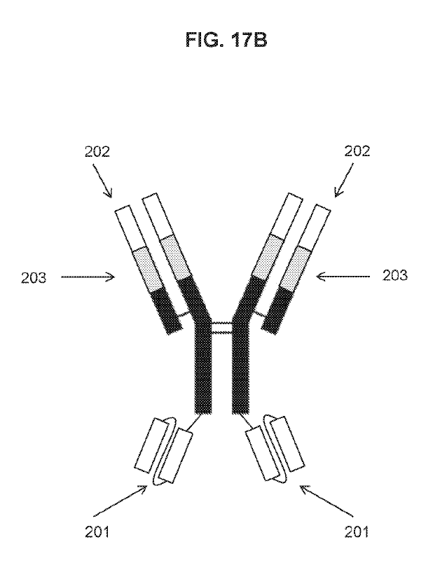

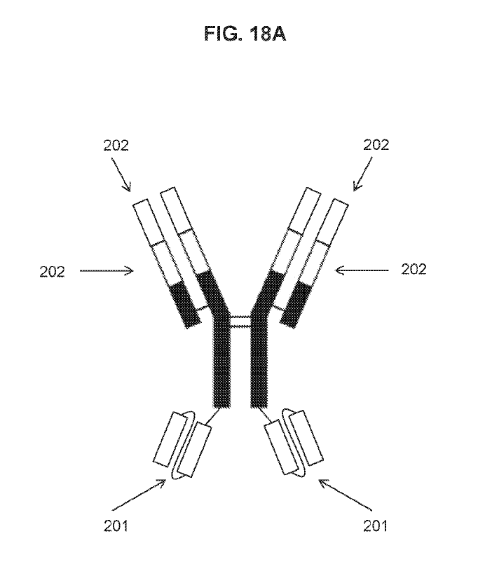

In FIG. 17A-17B, two ABM1 (201) scFvs are attached to the C-terminus of the heavy chains of IgG-like molecule. The N-terminal region of the IgG-like molecule comprises V_{H}-V_{L} regions forming two ABM2 binding sites (202), and V_{H}-V_{L} regions forming two ABM3 binding sites (203). In the embodiment depicted in FIG. 17A, the ABM3 binding sites are the most N-terminal ABM binding sites formed by the IgG-like molecule. In the embodiment depicted in FIG. 17B, the ABM2 binding sites are the most N-terminal ABM binding sites formed by the IgG-like molecule. It is also possible to attach one or more scFv ABM1 (201) to a C- or N-terminus of a light chain of the IgG-like molecule, or to an N-terminus of the heavy chains.

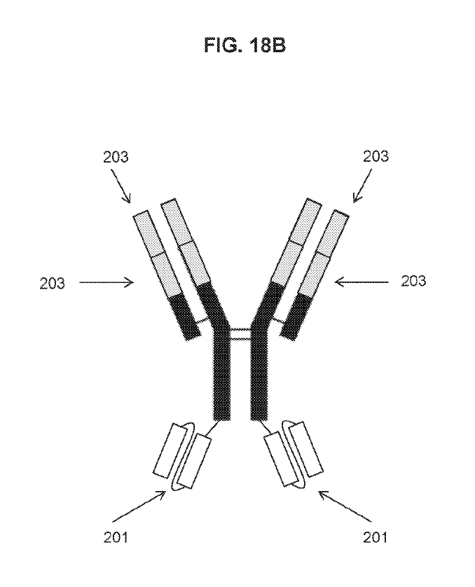

In FIG. 18A-18B, two ABM1 (201) scFvs are attached to the C-terminus of the heavy chains of an IgG-like molecule. The N-terminal region of the IgG-like molecule comprises V_{H}—V_{L} regions forming four ABM2 binding sites (202; FIG. 18A) or four ABM3 binding sites (203; FIG. 18B). It is also possible to attach one or more scFv ABM1 (201) to a C- or N-terminus of a light chain of the IgG-like molecule, or to an N-terminus of the heavy chains.

### 4. Preparation of Multispecific Immunomodulatory Antigen-Binding Constructs (MIACs)

MIACs can be prepared using nucleic acid cloning, protein expression, and protein assembly techniques known in the art, and described in more detail below. As described elsewhere in this disclosure, the ABMs that form the MIACs can be assembled from proteins (and fusion proteins) with multiple subunits. Where the MIAC is formed from multiple subunits, final assembly of the MIAC can be performed inside a recombinant cell or outside of a recombinant cell.

4.1. Antigen Preparation

The antigens used for production of the ABMs can be intact molecules expressed by cells (e.g., cancer-cell specific antigens, activating receptors, and/or inhibitory receptors), or fragments of these molecules. The antigens can be in form of isolated proteins, or in the form of cells expressing the proteins. Other forms of antigens useful for generating ABMs will be apparent to those skilled in the art.

4.2. Antibodies

Antibodies can be obtained, for example, using the hybridoma method first described by Kohler et al., Nature, 1975, 256:495-497, incorporated by reference in its entirety, and/or by recombinant DNA methods (see e.g., U.S. Pat. No. 4,816,567, incorporated by reference in its entirety). Monoclonal antibodies can also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing antibodies that will specifically bind to the protein used for immunization. Alternatively, lymphocytes can be immunized in vitro. Lymphocytes are then fused with myeloma cells using a suitable fusing agent, such as polyethylene glycol, to form a hybridoma cell. See Goding J. W., Monoclonal Antibodies: Principles and Practice, 3rd ed. (1986) Academic Press, San Diego, Calif., which is incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, Calif.), and SP-2 or X63-Ag8-653 cells (available from the American Type Culture Collection, Rockville, Md.). Human myeloma and mouse-human heteromyeloma cell lines also have been described for the production of human monoclonal antibodies. See e.g., Kozbor, J. Immunol., 1984, 133:3001, which is incorporated by reference in its entirety.

After the identification of hybridoma cells that produce antibodies of the desired specificity, affinity, and/or biological activity, selected clones can be subcloned by limiting dilution procedures and grown by standard methods. See Goding, supra. Suitable culture media for this purpose include, for example, D-MEM or RPMI-1640 medium. In addition, the hybridoma cells can be grown in vivo as ascites tumors in an animal.

DNA encoding the monoclonal antibodies can be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA can be placed into expression vectors, which are then transfected into host cells such as bacteria (e.g., E. coli), yeast (e.g., Saccharomyces or Komagataella (Pichia) sp.), COS cells, Chinese hamster ovary (CHO) cells, or myeloma cells that do not otherwise produce antibody, to produce the monoclonal antibodies.

4.2.1. Humanized Antibodies

Humanized antibodies can be generated by replacing most, or all, of the structural portions of a monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. USA., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089; 5,693,761; 5,693,762; and 6,180,370; each of which is incorporated by reference in its entirety.

4.2.2. Human Antibodies

Human antibodies can be generated by a variety of techniques known in the art, for example by using transgenic animals (e.g., humanized mice). See, e.g., Jakobovits et al., Proc. Natl. Acad. Sci. USA., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905); each of which is incorporated by reference in its entirety. Human antibodies can also be generated by in vitro activated B cells (see e.g., U.S. Pat. Nos. 5,567,610 and 5,229,275, each of which is incorporated by reference in its entirety). Human antibodies can also be derived from yeast-based libraries (see e.g., U.S. Pat. No. 8,691,730, which is incorporated by reference in its entirety.).

4.3. Alternative Scaffolds

Alternative scaffolds can be prepared by any method known in the art.

For example, methods of preparing Adnectins™ are described in Emanuel et al., mAbs, 2011, 3:38-48, incorporated by reference in its entirety. Methods of preparing iMabs are described in U.S. Pat. Pub. No. 2003/0215914, incorporated by reference in its entirety. Methods of preparing Anticalins® are described in Vogt and Skerra, Chem. Biochem., 2004, 5:191-199, incorporated by reference in its entirety. Methods of preparing Kunitz domains are described in Wagner et al., Biochem. & Biophys. Res. Comm., 1992, 186:118-1145, incorporated by reference in its entirety. Methods of preparing thioredoxin peptide aptamers are provided in Geyer and Brent, Meth. Enzymol., 2000, 328:171-208, incorporated by reference in its entirety. Methods of preparing Affibodies are provided in Fernandez, Curr. Opinion in Biotech., 2004, 15:364-373, incorporated by reference in its entirety. Methods of preparing DARPins are provided in Zahnd et al., J. Mol. Biol., 2007, 369:1015-1028, incorporated by reference in its entirety. Methods of preparing Affilins are provided in Ebersbach et al., J. Mol. Biol., 2007, 372:172-185, incorporated by reference in its entirety. Methods of preparing Tetranectins are provided in Graversen et al., J. Biol. Chem., 2000, 275:37390-37396, incorporated by reference in its entirety. Methods of preparing Avimers are provided in Silverman et al., Nature Biotech., 2005, 23:1556-1561, incorporated by reference in its entirety.

Further information on alternative scaffolds is provided in Binz et al., Nat. Biotechnol., 2005 23:1257-1268; and Skerra, Current Opin. in Biotech., 2007 18:295-304, each of which is incorporated by reference in its entirety.

4.4. Fusion Proteins

Fusion proteins can be produced using standard molecular biology methods known in the art. Generally, a polynucleotide sequence is synthesized that encodes two or more proteins that are to be fused, for example, two or more ABMs. The polynucleotide sequence is designed so that transcription and translation of the polynucleotide sequence results in expression of a polypeptide chain comprising the two or more proteins (e.g., ABMs). This polypeptide chain is referred to as the “fusion protein.” One illustrative example of a fusion protein is an IgG heavy chain fused to an scFv.

The polynucleotide sequence encoding the fusion protein can be designed so that the proteins to be fused are directly attached to each other, or attached to each other via an amino acid or polypeptide linker. In some embodiments, the N-terminus of one protein in the fusion protein directly follows the C-terminus of another protein in the fusion protein. In some embodiments, there is a single amino acid between the at least two proteins that comprise the fusion protein. In some embodiments, there is a polypeptide linker between the at least two proteins that comprise the fusion protein.

Methods for producing immunoglobulins with scFvs fused to their heavy chains are provided, for example, in Coloma and Morrison, Nature Biotechnol., 1997, 15:159-163, incorporated by reference in its entirety. Methods for producing immunoglobulins with scFvs fused to their light chains are provided, for example, in Orcutt et al., Protein Eng., 2010, 23:221-228, incorporated by reference in its entirety.

Methods for producing antibody fusion proteins in yeast and filamentous fungi are provided, for example, in Joosten et al., Microbial Cell. Factories, 2003, 2:1; and Powers et al., J. Immunol. Meth., 2001, 251:123-136; each of which is incorporated by reference in its entirety. Methods for producing scFv-Fc fusion proteins are described, for example, in Ono et al., J. Biosci. & Bioeng., 2003, 95:231-238; and Kamihara et al., J. Virology, 2005, 79:10864-10874; each of which is incorporated by reference in its entirety. Cell-free methods for the production of scFv fusion proteins are described, for example, in Kanter et al., Blood, 2007, 109:3393-3399, incorporated by reference in its entirety.

4.5. Polypeptide Linkers

Any suitable polypeptide linkers can be used to attach the ABMs of a MIAC to each other, and a skilled artisan is capable of selecting appropriate polypeptide linkers based on the nature of the ABMs and the applications of the MIAC. Suitable linker compositions are known in the art and described elsewhere in this disclosure. Selection of suitable linker lengths can be performed by evaluating the effects of different linker lengths on the affinity of the ABMs and the biological activity of the MIACs. Determination of suitable linker lengths is well within the capabilities of one of skill in the art.

As one illustrative example, in some embodiments, a suitable linker length can be determined by testing five linkers, with the following compositions and lengths: (2) (GGGGS) (SEQ ID NO:25); (2) (GGGGS)₂ (SEQ ID NO:26); (3) (GGGGS)₃ (SEQ ID NO:23); (4) (GGGGS)₄ (SEQ ID NO:24); and (5) (GGGGS)₅ (SEQ ID NO:27). Each of the five linkers can be evaluated as linkages between each of the ABMs of a MIAC, and the linkages that provide the best ABM affinity, MIAC bioactivity, yield, or the like can be selected for inclusion in the MIAC. The foregoing linkers are provided only for purposes of illustration and, as one of skill in the art will recognize, the composition and the length of the evaluated linkers can vary in any suitable manner.

In some embodiments, the polypeptide linkers are encoded by a polynucleotide that also encodes two or more ABMs linked by the polypeptide linker (e.g., a fusion protein). Such polynucleotides can be produced by assembling or synthesizing a polynucleotide encoding a first ABM, a first polypeptide linker, and a second ABM. In some embodiments, the polynucleotide can further encode a second polypeptide linker and a third ABM. The polynucleotide can then be expressed, according to the methods provided herein and known in the art, to produce a fusion protein comprising two or more ABMs connected by the linker.

In some embodiments, the ABMs are expressed separately and the polypeptide linker is used to attach two or more ABMs to each other after expression. In such embodiments, a first ABM is contacted with a first polypeptide linker under conditions suitable for the formation of a chemical bond between the first ABM and the first polypeptide linker. A second ABM is then contacted with the conjugate formed by the first ABM and the first polypeptide linker, under conditions suitable for the formation of a chemical bond between the first polypeptide linker and the second ABM. Additional ABMs can be conjugated to the first and/or second ABMs, or to the first linker, by utilizing similar techniques. Conditions suitable for the formation of chemical bonds between polypeptide linkers and ABMs are provided, for example, in Hermanson, Bioconjugate Techniques, 2013, 3d ed., Academic Press, London, UK, Waltham Mass., and San Diego, Calif., which is incorporated by reference in its entirety.

4.6. Chemical Coupling

In some embodiments, the ABMs are expressed separately and a chemical coupling reagent other than a polypeptide linker is used to attach two or more ABMs to each other after expression. In such embodiments, a first ABM is contacted with a first chemical coupling reagent under conditions suitable for the formation of a chemical bond between the first ABM and the first chemical coupling reagent. A second ABM is then contacted with the conjugate formed by the first ABM and the first chemical coupling reagent, under conditions suitable for the formation of a chemical bond between the first chemical coupling reagent and the second ABM. Additional ABMs can be conjugated to the first and/or second ABMs, or to the first chemical coupling reagent, by utilizing similar techniques. Conditions suitable for the formation of chemical bonds between chemical coupling reagents and ABMs are provided, for example, in Hermanson, Bioconjugate Techniques, 2013, 3d ed., Academic Press, London, UK, Waltham Mass., and San Diego, Calif., which is incorporated by reference in its entirety.

Any suitable coupling reagent can be used when chemically coupling MIACs. Coupling reagents include zero-length crosslinkers, homobifunctional crosslinkers, heterobifunctional crosslinkers, trifunctional crosslinkers, dendrimers and dendrons, chemoselective and bioorthogonal reagents, and the like. Illustrative suitable coupling reagents include, for example, m-maleimidobenzoic acid, N-hydroxysuccinimide ester, glutaraldehyde, and carbodiimides. Other suitable reagents for chemical coupling, and methods of their use, are described, for example, in Hermanson, Bioconjugate Techniques, 2013, 3d ed., Academic Press, London, UK, Waltham Mass., and San Diego, Calif., which is incorporated by reference in its entirety. Chemical coupling of antibodies to each other is described, for example, in Wong et al., Scand. J. Rheumatol., 2000, 29:282-287; Jung et al., Eur. J. Immunol., 1991, 21:2431-2435; Tuft et al., J. Immunol., 1991, 147:60-69; French, Methods Mol. Biol., 1998, 80:121-134; and Gavrilyuk et al., Bioorg. Med. Chem. Lett., 2009, 19:3716-3720; each of which is incorporated by reference in its entirety.

### 5. Vectors, Host Cells, and Methods for the Production of MIACs

5.1. Vectors

The invention also provides isolated nucleic acids encoding MIACs, vectors and host cells comprising the nucleic acids, and recombinant techniques for the production of the MIACs.

For recombinant production of the MIACs provided herein, the nucleic acid(s) encoding the MIAC can be isolated and inserted into a replicable vector for further cloning (i.e., amplification of the DNA) or expression. In some aspects, the nucleic acid can be produced by homologous recombination, for example as described in U.S. Pat. No. 5,204,244, which is incorporated by reference in its entirety.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615, which is incorporated by reference in its entirety.

5.2. Host Cells

Any suitable host cell can be used to produce the MIACs provided herein. Illustrative examples of suitable host cells are provided below. These host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for MIAC-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Komagataella (Pichia) pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the MIACs provided herein can be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 can be used. Each of the references cited in this paragraph is incorporated by reference in its entirety.

Any of these media can be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements can also be included at appropriate concentrations that would be known to those skilled in the art.

The culture conditions, such as temperature, pH, and the like, are those previously used with the host cell selected for expression, and will be apparent to the ordinarily skilled artisan.

5.3. Production of MIACs

General methods of recombinant protein production, and in particular, methods of antibody production can be applied to the production of the MIACs provided herein. Further details on methods of antibody production are provided in Al-Rubeai, ed., Antibody Expression and Production, 2011, Springer, Heidelberg, London, New York.

For example, in some embodiments, the MIAC is produced intracellularly, in the periplasmic space, or directly secreted into the medium. If the MIAC is produced intracellularly, as a first step, the particulate debris, either host cells or lysed fragments, is removed, for example, by centrifugation or ultrafiltration. For example, Carter et al. (Bio/Technology, 1992, 10:163-167; incorporated by reference in its entirety) describes a procedure for isolating ABMs which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the MIAC is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the MIAC can be useful, for example, where the MIAC accumulates in a cell as an insoluble aggregate, or where yields from periplasmic expression are low.

In some embodiments, the MIACs provided herein are expressed assembled by the cell. In some embodiments, the cell is a yeast cell that is capable of secreting a fully-formed, properly-assembled MIAC. In some embodiments, the MIACs provided herein require further treatment (e.g., the formation of disulfide linkages between heavy and/or light chains) in order to complete assembly. Assembly of functional ABMs from recombinantly-expressed and purified immunoglobulin heavy- and light-chains is described, for example, in Boss et al., Nucleic Acids Res., 1984, 12:3791-3806, incorporated by reference in its entirety. A skilled artisan will recognize that such techniques, among others described in this disclosure, could be readily adapted to assemble fusion proteins comprising an immunoglobulin heavy chain and/or fusion proteins comprising an immunoglobulin light chain.

Where the MIAC is secreted into the medium, supernatants from such expression systems are generally first concentrated using a commercially available protein concentration filter, for example, an Amicon® or Millipore® Pellcon® ultrafiltration unit. A protease inhibitor such as PMSF can be included in any of the foregoing steps to inhibit proteolysis and antibiotics can be included to prevent the growth of adventitious contaminants.

The MIAC composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the MIAC. Protein A can be used to purify MIACs that are based on human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the MIAC comprises a CH3 domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the MIAC of interest and contaminants can be subjected to low pH hydrophobic interaction chromatography using an elution buffer at a pH between about 2.5 to about 4.5, generally performed at low salt concentrations (e.g., from about 0 to about 0.25 M salt).

The individual components of the MIACs described herein can, in some cases, assemble to form a plurality of molecular species. If this occurs, the desired molecular species can be purified from the plurality using standard techniques, such as chromatography.

Furthermore, techniques such as the “knobs and holes” approach can be used to favor the production of properly assembled MIACs, which will reduce or eliminate the need for the removal of undesired molecular species. See U.S. Pat. Nos. 5,731,168; 7,695,936; 8,642,745; and 8,679,785; each of which is incorporated by reference in its entirety.

In some embodiments, it can be useful to generate ABMs that recognize more than one antigen. Any suitable technique can be used to generate such ABMs, including the knobs and holes approach described above. In some embodiments, ABMs that recognize more than one antigen are developed by isolating single domain antibodies that recognize different antigens and then combining them into a molecule that recognizes each of the different antigens recognized the by the single domain antibodies. For example, in some embodiments a single light chain antibody binding one antigen and a single heavy chain antibody binding another antigen are combined to form an intact immunoglobulin, or fragment thereof, that binds both antigens.

Multispecific ABMS can also be produced using the Azymetric™ platform, from Zymeworks Inc. Such multispecific ABMs, and methods of making the same, are described, for example, in U.S. Pat. Pub. Nos. 2012/0149876; 2012/0244577; 2013/0195849; 2013/0336973; 2014/0051835; 2014/0066378; 2014/0072581; and 2014/0200331; each of which is incorporated by reference in its entirety.

### 6. Conjugates

In some embodiments, the MIACs can be conjugated to an agent. Useful agents include, for example, therapeutic and diagnostic agents. In some embodiments, the agent is conjugated to the MIAC with a linker. In some aspects, the linker is a biodegradable linker.

Suitable agents and linkers can be selected by one of skill in the art. More information on linkers and agents can be found, for example, in Gerber et al., Nat. Prod. Rep., 2013, 30:625-639; Alley et al., Current Opinion in Chemical Biology, 2010, 14:529-537; and U.S. Pat. No. 5,010,176; each of which is incorporated by reference in its entirety. Additional therapeutic agents are discussed below, and can also be conjugated to the MIACs provided herein.

In some embodiments, an ABM of a MIAC provided herein comprises a masking moiety, and a cleavable moiety. The masking moiety inhibits the binding of the ABM to its target. The inhibitory activity of the masking moiety is reduced or eliminated after cleavage of the cleavable moiety. Such ABMs exhibit an “activatable” conformation such that the ABMs are less accessible to a target when the cleavable moiety is uncleaved, and become more accessible to a target after cleavage of the cleavable moiety in the presence of a cleaving agent. Examples of activatable binding polypeptides utilizing a masking moiety and cleavable moiety, and methods of obtaining them, are provided in U.S. Pat. Pub. Nos. 2010/0189651; 2010/0221212; 2013/0101555; 2013/0309230; 2013/0315906; 2014/0023664; 2014/0045195; 2014/0024810; 2014; 0235467; 2014/0255313; and U.S. Pat. Nos. 8,399,219; 8,513,390; 8,518,404; 8,541,203; 8,529,898; 8,563,269; each of which is incorporated by reference in its entirety.

### 7. Pharmaceutical Compositions and Methods of Administration

The MIACs provided herein can be provided in any appropriate pharmaceutical composition and be administered by any suitable route of administration. Suitable routes of administration include, but are not limited to, the parenteral, inhalation, intraarterial, intradermal, intramuscular, intraperitoneal, intravenous, nasal, pulmonary, and subcutaneous routes.

The pharmaceutical composition can comprise one or more pharmaceutical excipients. Any suitable pharmaceutical excipient can be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.), 6th Ed. (2009), incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical composition comprises an anti-foaming agent. Any suitable anti-foaming agent can be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a cosolvent. Illustrative examples of cosolvents include ethanol, poly(ethylene) glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene(glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that can be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical compositions are in a particulate form, such as a microparticle or a nanoparticle. Microparticles and nanoparticles can be formed from any suitable material, such as a polymer or a lipid. In some aspects, the microparticles or nanoparticles are micelles, liposomes, or polymersomes. In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic MIACs.

Further encompassed herein are anhydrous pharmaceutical compositions and dosage forms comprising a MIAC, since water can facilitate the degradation of some MIACs.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

7.1. Parenteral Dosage Forms

In certain embodiments, provided are parenteral dosage forms comprising MIACs. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the MIACs disclosed herein can also be incorporated into the parenteral dosage forms.

### 8. Assays

The activity of the MIACs provided herein can be evaluated using any suitable in vitro or in vivo assay for activity. In some embodiments, these assays can be adapted to high throughput approaches, to enable the efficient interrogation of large numbers of MIAC constructs.

In some embodiments, an assay measures proliferation of an effector cell. Any suitable assay can be used to measure proliferation of an effector cell. Suitable assays for measuring the proliferation of an effector cell include, for example, 3H-thymidine incorporation, the CFSE (carboxyfluorescein succinimidyl ester) dilution assay, and antibody detection of Ki67 antigen expression. See Gong and Klingemann, Leukemia, 1994, 8:652-658; Parish, Immunol. Cell Biol., 1999, 77:499-508; and Lyons, J. Immunol. Methods, 2000, 243:147-154, and Soares, J. Immunol. Methods, 2010, 362(1-2):43-50; each of which is incorporated by reference in its entirety.

In some embodiments, an assay measures stimulation and/or inhibition of effector cells. In some embodiments, activation and/or inhibition of effector cells can be assessed by measuring cytokine and/or chemokine production. The production of any suitable cytokine and/or chemokine can be measured. In some embodiments, the production of one or more cytokines and/or chemokines selected from IFN-gamma, TNF-alpha, IL-2, IL-4, IL-6, IL-8, IL-10, IL-12, IL-12p70, IL-15, MIP-alpha/beta, RANTES is measured. Any suitable assay can be used to measure the production of cytokines and/or chemokines. Suitable assays include, for example, the BD™ Cytometric Bead Array (CBA) assays (BD Biosciences), Luminex® xMAP® technology (Luminex Corporation), and ELISpot assays (e.g., Mabtech, ProImmune).

In some embodiments, the assay measures cytotoxic activity of an effector cell. Any suitable assay can be used to measure the cytotoxic activity of an effector cell. Suitable assays for measuring the cytotoxic activity of an effector cell include, for example, a Chromium-51 release assay, a granzyme B ELISpot or Luminex-based assay, measurement of CD107a cell surface mobilization, a caspase-3 assay, a flow cytometric assay with fluorophores PKH-26 and TO-PRO-3 iodide, and ADCC assays, such as the ADCC Reporter Bioassay (Promega). See Shafer-Weaver et al., J. Transl. Med., 2003, 1:14; Rininsland et al., J. Immunol. Methods, 2000, 240:143-155; Betts et al., J. Immunol. Methods, 2003, 28:65-78; Jerome et al., Apoptosis, 2003, 8:563-571; He et al., J. Immunol. Methods, 2005, 304:43-59; Lee-MacAry et al., J. Immunol. Methods, 2001, 252:83-92; and Aktas et al., Cell. Immunol., 2009, 254:149-154.

In some embodiments, the assay measures the fitness of an effector cell. Any suitable assay can be used to measure the fitness of an effector cell. Suitable assays for measuring the fitness of an effector cell include, for example, assays for apoptosis. Any suitable assay can be used to measure apoptosis, including an annexin V assay, and an annexin V FITC assay (e.g., BD Biosciences). See Vermes et al., J. Immunol. Methods, 1995, 84:39-51; and Poggi et al., J. Immunol., 2005, 17:2653-2660.

### 9. Dosage and Unit Dosage Forms

In human therapeutics, the doctor will determine the posology which he considers most appropriate according to a preventive or curative treatment and according to the age, weight, condition and other factors specific to the subject to be treated.

The amount of the MIAC which will be effective in the prevention or treatment of a disorder or one or more symptoms thereof will vary with the nature and severity of the disease or condition, and the route by which the MIAC is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses can be extrapolated from dose-response curves derived from in vitro or animal model test systems.

In certain embodiments, exemplary doses of a composition include milligram or microgram amounts of the MIAC per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiment, the dosage of the MIAC provided herein, based on weight of the MIAC, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the MIAC provided herein administered to prevent, treat, manage, or ameliorate a disorder, or one or more symptoms thereof in a subject is 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

The dose can be administered according to a suitable schedule, for example, once, two times, three times, or four times weekly; once, two times, three times, or four times monthly; or once, twice, three times, four times, five times, six times, seven times, eight times, nine times, ten times, eleven times, or twelve times yearly. It can be necessary to use dosages of the MIAC outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts can be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the MIACs provided herein are also encompassed by the herein described dosage amounts and dose frequency schedules. Further, when a subject is administered multiple dosages of a composition provided herein, not all of the dosages need be the same. For example, the dosage administered to the subject can be increased to improve the prophylactic or therapeutic effect of the composition or it can be decreased to reduce one or more side effects that a particular subject is experiencing.

In certain embodiments, treatment or prevention can be initiated with one or more loading doses of a MIAC or composition provided herein followed by one or more maintenance doses.

In certain embodiments, a dose of a MIAC or composition provided herein can be administered to achieve a steady-state concentration of the MIAC in blood or serum of the subject. The steady-state concentration can be determined by measurement according to techniques available to those of skill or can be based on the physical characteristics of the subject such as height, weight and age.

In certain embodiments, administration of the same composition can be repeated and the administrations can be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent can be repeated and the administration can be separated by at least 1 day, 2 days, 3 days, 5 days, 10 days, 15 days, 30 days, 45 days, 2 months, 75 days, 3 months, or 6 months.

In certain embodiments, the MIACs provided herein can be administered concurrently with a further therapeutic agent. Illustrative further therapeutic agents include, for example, alkylating agents (e.g., bendamustine, busulfan, carmustine, chlorambucil, cyclophosphamide, dacarbazine, ifosfamide, melphalan, procarbazine, streptozocin, temozolomide); anti-metabolites (e.g., asparaginase, capecitabine, cytarabine, 5-fluoro uracil, fludarabine, gemcitabine, methotrexate, pemetrexed, raltitrexed); anti-tumor antibiotics (e.g., actinomycin D, bleomycin, daunorubicin, doxorubicin, epirubicin, idarubicin, mitomycin, mitoxantrone); microtubule inhibitors (e.g., etoposide, docetaxel, irinotecan, paclitaxel, topotecan, vinblastine, vincristine, vinorelbine); DNA linking agents (e.g., carboplatin, cisplatin, oxaliplatin); biologic agents (e.g., alemtuzumab, bevacizumab, brentuximab, cetuximab, denosumab, ibritumomab, interferons, ipilimumab, obinutuzumab, ofatumumab, panitumumab, ramucirumab, rituximab, ruxolitinib, siltuximab, tositumomab, trastuzumab); kinase inhibitors (e.g., afatinib, axitinib, bosutinib, crizotinib, dabrafenib, erlotinib, gefitinib, ibrutinib, imatinib, lapatinib, nilotinib, pazopanib, ponatinib, regorafenib, ruxolitinib, sorafenib, sunitinib, trametinib, vandetanib, vemurafenib); rapamycin derivatives (e.g., rapamycin, sirolimus, temsirolimus, everolimus, deforolimus); bisphosphonates (e.g., clodronate, ibandronic acid, pamidronate, zoledronic acid); and hormones and other drugs (e.g., anastrozole, abiraterone, amifostine, bexarotene, bicalutamide, buserelin, cyproterone, degarelix, exemestane, flutamide, folinic acid, fulvestrant, goserelin, lanreotide, lenalidomide, letrozole, leuprorelin, medroxyprogesterone, megestrol, mesna, octreotide, stilboestrol, tamoxifen, thalidomide, triptorelin).

Additional examples of further therapeutic agents useful with the MIACs provided herein include abiraterone, aldesleukin, aminolevulinic acid, aprepitant, anastrozole, bendamustine, bexarotene, bicalutamide, bleomycin, bortezomib, busulfan, cabazitaxel, cabozantinib-S-malate, capecitabine, carboplatin, carfilzomib, chlorambucil, clofarabine, cisplatin, cytarabine, dactinomycin, dabrafenib, dacarbazine, dasatinib, decitabine, degarelix, denileukin diftitox, dexrazoxane, docetaxel, eltrombopag, enzalutamide, epirubicin, eribulin, erlotinib, exemestane, filgrastim, fludarabine, fluorouracil, fulvestrant, gemcitabine, glucarpidase, goserelin, ifosfamide, imiquimod, interferon alfa-2b, irinotecan, ixabepilone, lenalidomide, letrozole, leucovorin, leuprolide, lomustine, mechlorethamine, megestrol, mercaptopurine, mesylate, mitomycin C, navelbine, nelarabine, omacetaxine, oxaliplatin, paclitaxel, palifermin, palonosetron, pamidronate, pemetrexed, plerixafor, pomalidomide, ponatinib, pralatrexate, procarbazine, raloxifene, rasburicase, romidepsin, romiplostim, temozolomide, topotecan, toremifene, vinblastine, vincristine, vinorelbine, vorinostat, vismodegib, and zoledronic acid.

These agents can be co-administered with the MIACs provided herein, or administered according to any suitable schedule. In some embodiments, the agents are formulated in a unit dosage form with a MIAC provided herein. In some embodiments, the agents are conjugated to a MIAC provided herein.

### 10. Therapeutic Applications

For therapeutic applications, the MIACs of the invention are administered to a mammal, generally a human, in a pharmaceutically acceptable dosage form such as those known in the art and those discussed above. For example, the MIACs of the invention can be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The MIACs also are suitably administered by peritumoral, intralesional, or perilesional routes, to exert local as well as systemic therapeutic effects. The intraperitoneal route can be particularly useful, for example, in the treatment of ovarian tumors.

The MIACs provided herein can be useful for the treatment of any disease or condition in which targeted effector cell activation is of therapeutic benefit, such as cancer.

Any suitable cancer can be treated with the MIACs provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In some embodiments, provided herein is a method of treating a subject with a disease, comprising administering an effective amount of a MIAC to the subject. In some embodiments, provided herein is a method of treating a subject with a disease, comprising administering an effective amount of a pharmaceutical composition comprising a MIAC to the subject. In some aspects, the disease is cancer.

In some embodiments, provided herein is a method of killing a cancer cell, comprising contacting the cancer cell with a MIAC, wherein the MIAC activates an effector cell that kills the cancer cell. In some embodiments, the method is an in vitro method. In some embodiments, the method is an in vivo method.

Also provided are methods of activating an effector cell comprising contacting the effector cell with a MIAC, wherein the MIAC activates the effector cell. In some embodiments, the method is an in vitro method. In some embodiments, the method is an in vivo method. In some aspects, the method is carried out in proximity to a cancer cell. In some aspects, the proximity is close enough for the effector cell to exert cytotoxic activity against the cancer cell.

### 11. Diagnostic Applications

In some embodiments, the MIACs provided herein are used in diagnostic applications. For example, a MIAC can be labeled with a detectable moiety. Suitable detectable moieties include, but are not limited to radioisotopes, fluorescent labels, and enzyme-substrate labels.

### 12. Kits

In some embodiments, a MIAC provided herein is provided in the form of a kit. In some embodiments, the kit comprises a packaged combination of reagents in predetermined amounts with instructions for performing a procedure. In some embodiments, the procedure is a therapeutic procedure. In other embodiments, the procedure is a diagnostic assay. In still other embodiments, the procedure is a research assay.

In some embodiments, the kit further comprises a solvent for the reconstitution of the MIAC. In some embodiments, the MIAC is provided in the form of a pharmaceutical composition. In some embodiments, the pharmaceutical composition is a lyophilized pharmaceutical composition.

### EXAMPLES

### Example 1: MIAC1: An IgG-scFv-scFv MIAC Targeting CD30, CD137, and Inhibitory KIR

A MIAC as described herein is made with the following antigen-binding modules. ABM1 binds CD30, a protein expressed in classical Hodgkin lymphoma and systemic anaplastic large cell lymphoma. ABM2 binds and agonizes CD137, an activating receptor expressed by effector cells. ABM3 binds and antagonizes inhibitory KIR receptors expressed by effector cells.

In this example, ABM1 is the IgG from brentuximab (Adcetris®). ABM2 is an scFv produced from urelumab (Bristol-Myers Squibb). ABM3 is an scFv produced from lirilumab (Innate Pharma). The scFvs are assembled by standard techniques, using a (GGGGS)₄ (SEQ ID NO: 24) linker between the heavy chain and the light chain. The scFvs are assembled in both orders (i.e., V_{H}-linker-V_{L} and V_{L}-linker-V_{H}). See Plückthun A. (1994). Antibodies from Escherichia coli. In Rosenberg M. & Moore G. P. (Eds.), The Pharmacology of Monoclonal Antibodies vol. 113 (pp. 269-315).

Standard molecular biology techniques are used to assemble nucleic acids encoding the three ABMs, and appropriate fusion proteins. The fusion proteins used in this example include (1) V_{H}-ABM2 fusions, (2) V_{H}-ABM3 fusions, (3) V_{L}-ABM2 fusions, and (4) V_{L}-ABM3 fusions.

In this example, there are two ABM1 sites, which are formed by the four variable domains (two V_{H} and two V_{L}) of the IgG. The number of ABM2 and ABM3 sites varies as described below. The MIAC of this example also contains an ABM4, formed by the Fc region of the IgG. As described elsewhere in this disclosure, this domain is capable of engaging effector cells, such as NK cells, that express an Fc receptor.

Nucleic acids encoding ten different types of MIACs (designated 1.1 to 1.10) are synthesized and transfected into CHO cells. The MIACs are expressed and purified using a protein A column. MIAC 1.1 is shown in FIG. 2A. MIAC 1.2 is shown in FIG. 2B. MIAC 1.3 is shown in FIG. 2C. MIAC 1.4 is shown in FIG. 2D. MIAC 1.5 is shown in FIG. 3A. MIAC 1.6 is shown in FIG. 3B. MIAC 1.7 is shown in FIG. 3C. MIAC 1.8 is shown in FIG. 3D. MIAC 1.9 is shown in FIG. 4A. MIAC 1.10 is shown in FIG. 4B.

The MIACs are tested in vitro and in vivo for their ability to activate NK cells and destroy cancer cells and tumors. In vitro assays include monitoring the activation of NK cells by measuring downregulation of CD16, upregulation of CD69, percentage of LAMP1+ cells, interferon gamma release, percentage of 7-AAD-positive cells, and proliferation after exposure to the MIACs, and killing of Raji cells exposed to effector cells in the presence of the MIACs. In vivo assays are performed by treating animals with induced tumors or tumor xenografts with the MIACs provided herein.

Proliferation of the effector cells is evaluated by 3H-thymidine. Stimulation of effector cells is measured by measuring cytokine and chemokine secretion, using the BD™Cytometric Bead Array (CBA) assay to measure the concentrations of IFN-gamma, TNF-alpha, IL-2, IL-4, IL-6, IL-8, IL-10, IL-12, IL-12p70, IL-15, MIP-alpha/beta, and RANTES. Cytotoxic activity is measured by a Chromium-51 release assay, a granzyme B ELISpot assay, measuring CD107a cell surface mobilization, a caspase-3 assay, and a flow cytometric assay with fluorophores PKH-26 and TO-PRO-3 iodide. Fitness of the effector cells is measured using an annexin V FITC assay.

### Example 2: MIAC2: An IgG-scFv-scFv MIAC Targeting CD20, NKG2D, and Inhibitory KIR

A MIAC as described herein is made with the following antigen-binding modules. ABM1 binds CD20, a protein expressed on the surface of B cells, and relevant to the treatment of B cell lymphomas and leukemias. An example of an ABM for CD20 is rituximab. ABM2 binds and agonizes NKG2D, an activating receptor expressed by effector cells. ABM3 binds and antagonizes inhibitory KIR receptors expressed by effector cells.

MIACs 2.1 to 2.10 (corresponding to the MIACs provided in FIGS. 2A to 4B) are synthesized and characterized as described in Example 1.

### Example 3: MIAC3: scFv-IgG-scFv and scFv-scFv-IgG MIACs Targeting CD20, NKG2D, and Inhibitory KIR

A MIAC as described herein is made with the following antigen-binding modules. ABM1 binds CD20, a protein expressed on the surface of B cells, and relevant to the treatment of B cell lymphomas and leukemias. ABM2 binds and agonizes NKG2D, an activating receptor expressed by effector cells. ABM3 binds and antagonizes inhibitory KIR receptors expressed by effector cells.

Two configurations of this MIAC are produced. In the first configuration, ABM1 is an scFv, ABM2 is an IgG, and ABM3 is an scFv (MIAC 3.1). An example of this MIAC is depicted in FIG. 5A. In the second configuration, ABM1 is an scFv, ABM2 is an scFv, and ABM3 is an IgG (MIAC 3.2). An example of this MIAC is depicted in FIG. 5B.

### Example 4: MIAC4: An scFv-scFv-scFv MIAC Targeting CD20, NKG2D, and Inhibitory KIR

A MIAC as described herein is made with the following antigen-binding modules. ABM1 binds CD20, a protein expressed on the surface of B cells, and relevant to the treatment of B cell lymphomas and leukemias. ABM2 binds and agonizes NKG2D, an activating receptor expressed by effector cells. ABM3 binds and antagonizes inhibitory KIR receptors expressed by effector cells.

In this MIAC, each of ABM1, ABM2, and ABM3 are scFvs. The MIAC is expressed as a fusion protein with ABM1 at the N-terminus, ABM2 in the middle, and ABM3 at the C-terminus. An example of this MIAC is depicted in FIG. 6.

### Example 5: MIAC5: An IgG-scFv MIAC Targeting CD20 and NKG2D

A MIAC as described herein is made with the following antigen-binding modules. ABM1 binds CD20, a protein expressed on the surface of B cells, and relevant to the treatment of B cell lymphomas and leukemias. ABM2 binds and agonizes NKG2D, an activating receptor expressed by effector cells.

MIACs 5.1 to 5.6 (corresponding to the MIACs provided in FIGS. 11A-B, 12A, 13A-B, and 14A) are synthesized and characterized as described in Example 1.

### Example 6: MIAC6: An IgG-scFv MIAC Targeting CD20 and Inhibitory KIR

A MIAC as described herein is made with the following antigen-binding modules. ABM1 binds CD20, a protein expressed on the surface of B cells, and relevant to the treatment of B cell lymphomas and leukemias. ABM3 binds and antagonizes an inhibitory KIR, an inhibitory receptor expressed by effector cells.

MIACs 6.1 to 6.6 (corresponding to the MIACs provided in FIGS. 11C-D, 12B, 13C-D, and 14B) are synthesized and characterized as described in Example 1.

### Example 7: MIAC7: scFv-IgG MIACs Targeting CD20 and NKG2D

A MIAC as described herein is made with the following antigen-binding modules. ABM1 binds CD20, a protein expressed on the surface of B cells, and relevant to the treatment of B cell lymphomas and leukemias. ABM2 binds and agonizes NKG2D, an activating receptor expressed by effector cells.

ABM1 is an scFv, one ABM2 is an IgG, and a second ABM2 is an scFv (MIAC 7.1). An example of this MIAC is depicted in FIG. 15A. The MIACs are synthesized and characterized as described in Example 1.

### Example 8: MIAC8: scFv-IgG MIACs Targeting CD20 and Inhibitory KIR

A MIAC as described herein is made with the following antigen-binding modules. ABM1 binds CD20, a protein expressed on the surface of B cells, and relevant to the treatment of B cell lymphomas and leukemias. ABM3 binds and antagonizes an inhibitory KIR, an inhibitory receptor expressed by effector cells.

ABM1 is an scFv, one ABM3 is an IgG, and a second ABM3 is an scFv (MIAC 7.1). An example of this MIAC is depicted in FIG. 15B. The MIAC is synthesized and characterized as described in Example 1.

### Example 9: MIAC9: An scFv-scFv-scFv MIAC Targeting CD20 and NKG2D

A MIAC as described herein is made with the following antigen-binding modules. ABM1 binds CD20, a protein expressed on the surface of B cells, and relevant to the treatment of B cell lymphomas and leukemias. ABM2 binds and agonizes NKG2D, an activating receptor expressed by effector cells.

In this MIAC, each of ABM1 and ABM2 is an scFv. The MIAC is expressed as a fusion protein with ABM1 at the N-terminus, ABM2 in the middle, and ABM2 at the C-terminus. An example of this MIAC (MIAC 9.1) is depicted in FIG. 16A. The MIAC is synthesized and characterized as described in Example 1.

### Example 10: MIAC10: An scFv-scFv-scFv MIAC Targeting CD20 and Inhibitory KIR

A MIAC as described herein is made with the following antigen-binding modules. ABM1 binds CD20, a protein expressed on the surface of B cells, and relevant to the treatment of B cell lymphomas and leukemias. ABM3 binds and antagonizes an inhibitory KIR, an inhibitory receptor expressed by effector cells.

In this MIAC, each of ABM1 and ABM3 is an scFv. The MIAC is expressed as a fusion protein with ABM1 at the N-terminus, ABM3 in the middle, and ABM3 at the C-terminus. An example of this MIAC (MIAC 10.1) is depicted in FIG. 16B. The MIAC is synthesized and characterized as described in Example 1.

### Example 11: MIAC11: An scFv-IgG-Based MIAC Targeting CD20, NKG2D, and Inhibitory KIR

A MIAC as described herein is made with the following antigen-binding modules. ABM1 binds CD20, a protein expressed on the surface of B cells, and relevant to the treatment of B cell lymphomas and leukemias. ABM2 binds and agonizes NKG2D, an activating receptor expressed by effector cells. ABM3 binds and antagonizes an inhibitory KIR, an inhibitory receptor expressed by effector cells.

In this MIAC, ABM1 is an scFv. ABMs 2 and 3 are the variable domains of an IgG-based molecule. In one embodiment (FIG. 17A; MIAC 11.1), the ABM3 binding site is the most N-terminal binding site on the IgG-based molecule. In another embodiment (FIG. 17B; MIAC 11.2), the ABM2 binding site is the most N-terminal binding site on the IgG-based molecule. The MIACs are synthesized and characterized as described in Example 1.

### Example 12: MIAC12: An scFv-IgG-Based MIAC Targeting CD20 and NKG2D or Inhibitory KIR

A MIAC as described herein is made with the following antigen-binding modules. ABM1 binds CD20, a protein expressed on the surface of B cells, and relevant to the treatment of B cell lymphomas and leukemias. ABM2 binds and agonizes NKG2D, an activating receptor expressed by effector cells. ABM3 binds and antagonizes an inhibitory KIR, an inhibitory receptor expressed by effector cells. The MIAC contains either ABM2 or ABM3, but not both.

In this MIAC, ABM1 is an scFv. ABMs 2 and 3 are the variable domains of an IgG-based molecule. In one embodiment (FIG. 18A; MIAC 12.1), the ABM2 binding sites are formed by the IgG-based molecule. In another embodiment (FIG. 18B; MIAC 12.2), the ABM3 binding sites are formed by the IgG-based molecule. The MIACs are synthesized and characterized as described in Example 1.

### Example 13: MIAC13: An scFv-IgG MIAC Targeting CD30, CD137, and PD-1

A MIAC as described herein is made with the following antigen-binding modules. ABM1 binds CD30, a protein expressed in classical Hodgkin lymphoma and systemic anaplastic large cell lymphoma. ABM2 binds and agonizes CD137, an activating receptor expressed by effector cells. ABM3 binds and antagonizes PD-1 an inhibitory KIR, an inhibitory receptor expressed by effector cells.

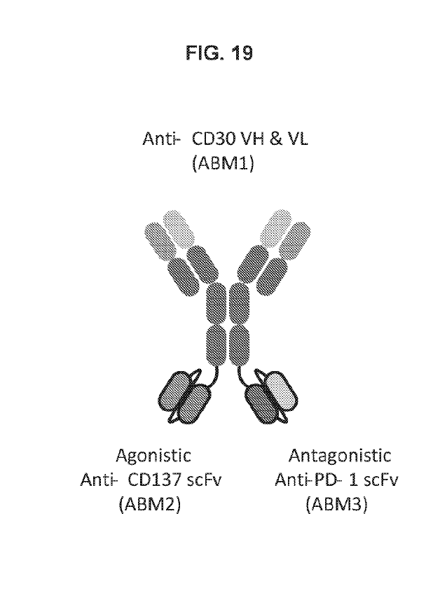

In this MIAC, ABM1 is an IgG. ABMs 2 and 3 are scFvs. The ABM 2 and 3 scFvs are attached to the C-termini of the heavy chains of the IgG forming ABM1, as illustrated in FIG. 19. The sequences for the two heavy chains and the light chain are provided in SEQ ID NOs: 1-3.

The MIACs are synthesized and characterized as described in Example 1.

### Example 14: MIAC14: An scFv-IgG-Based MIAC Targeting CD30, CD137, and PD-1

A MIAC as described herein is made with the following antigen-binding modules. ABM1 binds CD30, a protein expressed in classical Hodgkin lymphoma and systemic anaplastic large cell lymphoma. ABM2 binds and agonizes CD137, an activating receptor expressed by effector cells. ABM3 binds and antagonizes PD-1 an inhibitory KIR, an inhibitory receptor expressed by effector cells.

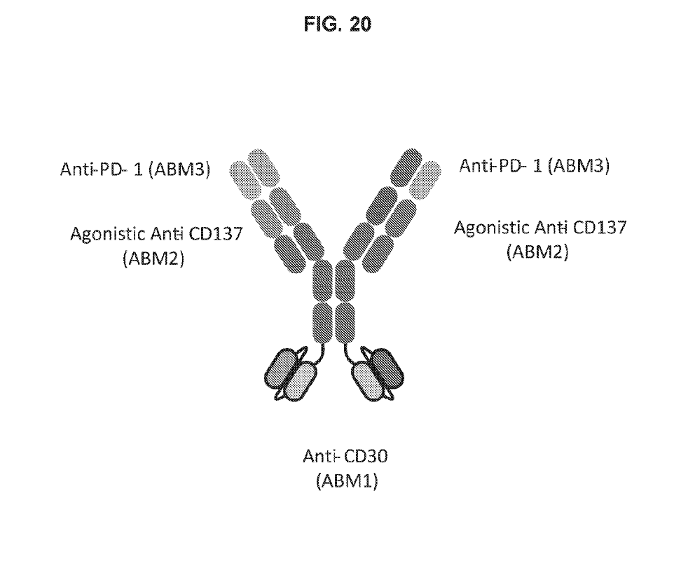

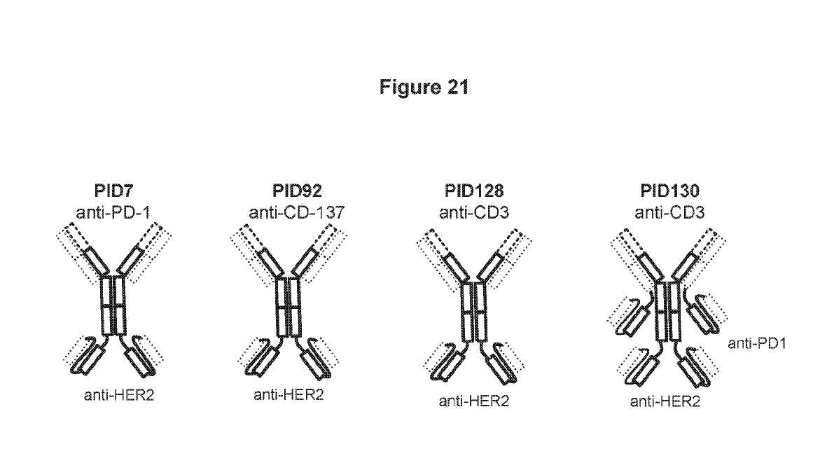

As depicted in FIG. 20, in this MIAC, two ABM1 scFvs are attached to the C-terminus of the heavy chains of an IgG-like molecule. The N-terminal region of the IgG-like molecule comprises V_{H}-V_{L} regions forming two ABM2 binding sites, and V_{H}-V_{L} regions forming two ABM3 binding sites. In this MIAC, the ABM3 binding sites are the most N-terminal ABMs formed by the IgG-like molecule. The sequences for the heavy chain and the light chain are provided in SEQ ID NOs: 5-6.

The MIACs are synthesized and characterized as described in Example 1.

### Example 15: Preparation of Exemplary Monospecific, Bispecific, and Trispecific MIAC Constructs

A number of exemplary monospecific, bispecific, and trispecific MIAC constructs targeting individual or combinations of tumor associated antigens, checkpoint receptors, and activating receptors were prepared as described below. Representations of exemplary antibody formats are shown in FIG. 21. For bispecific constructs, an scFv against target 1 was genetically fused to the C-terminus of the heavy chain of a full-length IgG1 antibody against target 2 through a (G₄S)₃ linker (SEQ ID NO: 23). For trispecific constructs, scFv's against target 1 and 2 were genetically fused to the C-termini of the heavy and light chains respectively of a full length IgG1 antibody against target 3 through (G₄S)₃ linkers (SEQ ID NO: 23). For constructs containing an anti-CD3 binding domain, amino acid N297 in the Fc domain was mutated to an alanine to prevent N-linked glycosylation and reduce ADCC.

### Exemplary Monospecific, Bispecific, and Trispecific Constructs

Exemplary monospecific, bispecific, and trispecific antibody constructs were prepared as shown in Tables A-C.

TABLE A
Exemplary monospecific proteins

| Protein | Specificity | HC plasmid | LC plasmid | Fab domain | Constant domain |
|---|---|---|---|---|---|
| PID-2 | PD-1 | DID-15 (SEQ | DID-16 (SEQ | Pembrolizumab | Human IgG1 |
| | | ID NO: 13) | ID NO: 14) | | |
| PID-3 | Her2 | DID-1 (SEQ | DID-2 (SEQ | Trastuzumab | Human IgG1 |
| | | ID NO: 7) | ID NO: 8) | | |
| PID-90 | CD137 | DID-9 (SEQ | DID-309 (SEQ | Urelumab | Human IgG1 |
| | | ID NO: 11) | ID NO: 12) | | |
| PID-127 | CD3 | DID-365 (SEQ | DID-8 (SEQ | Blinatumomab_CD3 | Aglycosylated |
| | | ID NO: 9) | ID NO: 10) | | human IgG1 |
| | | | | | (N297A mutation) |

TABLE B
Exemplary bispecific proteins

| | | | | N-terminal | Constant | C-terminal |
|---|---|---|---|---|---|---|
| Protein | Specificity | HC plasmid | LC plasmid | Fab domain | domain | scFv |
| PID-7 | Her2/PD-1 | DID-27 (SEQ | DID-16 (SEQ | Pembrolizumab | Human IgG1 | Trastuzumab |
| | | ID NO: 17) | ID NO: 14) | | | |
| PID-92 | Her2/CD137 | DID-26 (SEQ | DID-309 | Urelumab | Human IgG1 | Trastuzumab |
| | | ID NO: 16) | (SEQ ID NO: | | | |
| | | | 12) | | | |
| PID- | Her2/CD3 | DID-366 | DID-8 (SEQ | Blinatumomab_CD3 | Aglycosylated | Trastuzumab |
| 128 | | (SEQ ID | ID NO: 10) | | human IgG1 | |
| | | NO: 15) | | | (N297A | |
| | | | | | mutation) | |

TABLE C
Exemplary trispecific proteins

| | | | | | | HC C- | LC C- |
|---|---|---|---|---|---|---|---|
| | | | | N-terminal | Constant | terminal | terminal |
| Protein | Specificity | HC plasmid | LC plasmid | Fab domain | domain | scFv | scFv |
| PID- | Her2/CD137/PD-1 | DID-26 | DID-310 | Urelumab | Human IgG1 | Trastuzumab | Pembrolizumab |
| 98 | | (SEQ ID | (SEQ ID NO: | | | | |
| | | NO: 16) | 19) | | | | |
| PID- | Her2/CD3/PD-1 | DID-366 | DID-50 | Blinatumomab_CD3 | Aglycosylated | Trastuzumab | Pembrolizumab |
| 130 | | (SEQ ID | (SEQ ID NO: | | human | | |
| | | NO: 15) | 18) | | IgG1 | | |

Genes encoding the light and heavy chains for each monospecific, bispecific, and trispecific construct were synthesized by DNA2.0 (Menlo Park, Calif.) and cloned into their pD2610.v10 expression vector. Plasmids were scaled up and purified using the Qiagen Maxiprep Plus kit according to manufacturer's instructions.

### Example 16: Bench-Scale Expression and Purification of Exemplary Monospecific, Bispecific, and Trispecific MIAC Constructs

The final gene products were sub-cloned by DNA2.0 (Menlo Park, Calif.) into either the commercial mammalian expression vector pD2610-v1 or pD2610-v10 (DNA2.0, Menlo Park, Calif.) and expressed by transient transfection in a human embryonic kidney 293 (HEK-293) cell line.

The HEK-293 cells used for protein expression were those provided in the commercially available “Expi293 Expression System” from Thermo-Fisher Scientific. Transfections and expressions were performed as per the manufacturer's recommendations. Briefly, Expi293 cells were transfected at 2 to 3 million cells/ml during exponential growth with a lipid-DNA complex mix. The lipid transfection reagent used was provided in the “Expi293 Expression System Kit”, and known under the brand name “ExpiFectamine 293 Reagent”. The DNA used for transfection was prepared using an endotoxin-free DNA preparation kit, such as the Qiagen Endo-free Maxi kit. The final amount of DNA used for transfection was 1 ug DNA per ml of transfection culture volume, i.e. 30 ug of total DNA was used for a 30 ml expression volume. Since final proteins were composed of at least two protein sequences (namely, minimally, heavy and light chains), which were expressed individually from separate plasmids, the optimal ratio for plasmid combinations was determined. For example, to determine the optimal concentration range for forming heterodimers, the DNA was transfected in optimal DNA ratios of the heavy chain A (HC-A), light chain (LC), and heavy chain B (HC-B) that allow for heterodimer formation (e.g. HC-A/HC-B/LC ratios=40:40:20 (as was the case for PID94). Transfected cells were harvested after 4-5 days with the culture medium collected after centrifugation at 4000 rpm and clarified using a 0.45 μm filter.

The clarified culture medium was loaded onto a Pierce protein-A agarose packed column (Thermo-Fisher) and washed with 10 column volumes of PBS buffer at pH 7.2. The antibody was eluted with 10 column volumes of citrate buffer at pH 3.6 with the pooled fractions containing the antibody neutralized with TRIS at pH 8.0.

To characterize the quality of the protein-A eluted antibody reagents, samples were analyzed by analytical scale gel filtration (SEC). For this, 5 to 20 μg of protein was run on a YMC-Pack Diol-200 column (YMC Ltd, Kyoto, Japan) via an Agilent Technologies series 1200 HPLC using 20 mM sodium phosphate, 400 mM sodium chloride, pH 7.0 as the mobile phase. The chromatogram obtained was used to calculate the percentage of the peak of interest (POI) of assembled mono-, bi- and tri-specific antibody reagents.

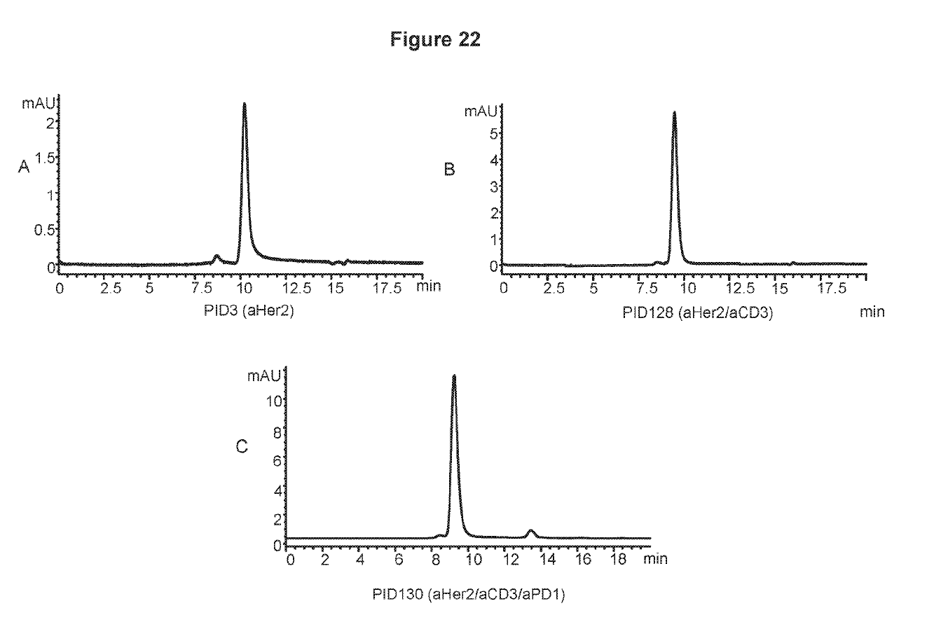

The protein-A antibody eluate was further purified as required by gel filtration (SEC). For gel filtration, approximately 1 mg of the antibody mixture was concentrated to 1 mL and loaded onto a Superose 6 Increase 10/300 GL column (GE Healthcare) via an AKTA Pure FPLC at a flow-rate of 1 mL/min. PBS buffer at pH 7.4 was used at a flow-rate of 1 mL/min. Fractions corresponding to the purified antibody were collected, concentrated to ˜1 mg/mL and filter sterilized through a 0.2 μm filter. The resultant material was characterized by analytical gel filtration, as described in the previous paragraph. Table D below exemplifies the percentage of POI for a number of the final antibody reagents generated. FIG. 22 depicts an analytical gel filtration chromatograph for a mono- (PID3), bi- (PID128) and tri-specific (PID130) antibody reagents.

TABLE D
Analytical gel filtration characterization

| Reagent | % POI |
|---|---|
| PID2 (PD-1) | 99.1 |
| PID3 (Her2) | 94.6 |
| PID90¹ (CD137) | 97.0 |
| PID127¹⁶ (CD3^{aglyco}) | 100.0 |
| PID92⁶ (Her2/CD137) | 95.0 |
| PID128²³ (Her2/CD3^{aglyco}) | 94.2 |
| PID130⁷⁵ (Her2/CD3^{aglyco}/PD-1) | 92.2 |

### Example 17: Antigen Binding Analysis of Exemplary Monospecific, Bispecific, and Trispecific MIAC Constructs by ForteBio Octet®

### Individual Antigen Binding

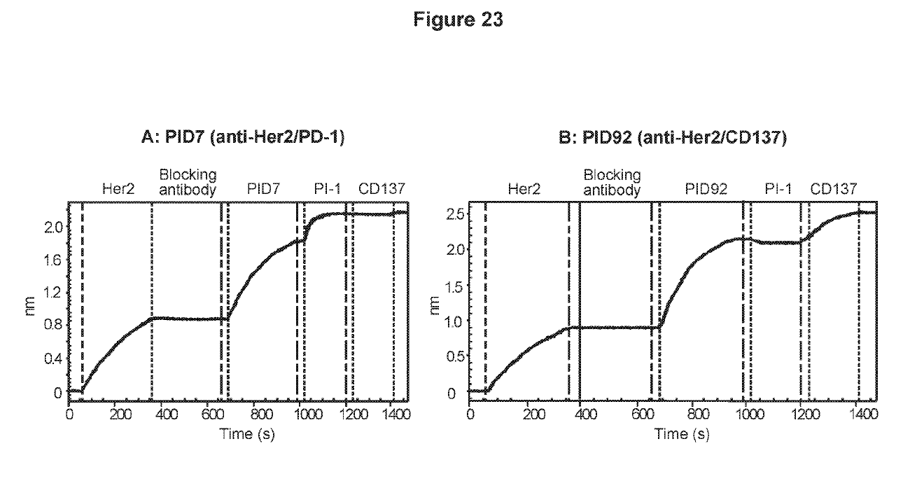

Antigen binding of MIAC constructs were analyzed using ForteBio Octet. Recombinant human PD-1 Fc, Her2 Fc, CD137 Fc fusion proteins were purchased from Sino Biological and recombinant Human CD3 epsilon His tagged was from AcroBiosystems. All antigens were reconstituted in PBS at the final concentration of 0.25 ug/ul. For the analysis of PD-1, Her2, and CD137 binding, antigens (100 nM in Fortebio Kinetics buffer) were immobilized on Protein A sensors for 3 minutes, potential free binding sites on the sensors were subsequently blocked by dipping the sensors in 300 nM rituximab for 10 minutes. Association proceeded by dipping sensors in 100 nM MIAC constructs for 5 minutes and disassociation occurred in blank kinetics buffer for 5 minutes. For CD3 epsilon binding, antigen was immobilized on Ni-NTA sensors; association was performed in 50 nM MIAC constructs. Binding data were analyzed using ForteBio Data Analysis 9.0 software. Representative binding sensograms are shown in FIG. 23. The measured affinities are summarized in Table E. This data shows that each of the four tested MIACs that bind more than one target can simultaneously bind each target. This data also shows that the MIAC binding affinities range from 0.12 to 1.66 nM under simultaneous binding conditions.

TABLE E
Binding affinities

| | | Affinity (nM) | | |
|---|---|---|---|---|
| PIDs | Targeted antigen | PD1 | Her2 | CD137 |
| PID2 | PD1 | 0.52 | NB | NB |
| PID3 | Her2 | NB | 0.88 | NB |
| PID90 | CD137 | NB | NB | 0.7 |
| PID7 | Her2/PD1 | 0.12 | 1.34 | NB |
| PID92 | A-Her2/α-CD137 | NB | 1.12 | 0.23 |
| PID128 | Her2/CD3 | NB | 4.2 | 5.5 |
| PID130 | Her2/CD3/PD1 | 0.37 | 1.66 | 9.6 |

NB: no binding detected

### Example 18: Analysis of Her2 and PD-L1 Expression in Human Breast Cancer Cell Lines by Flow Cytometry

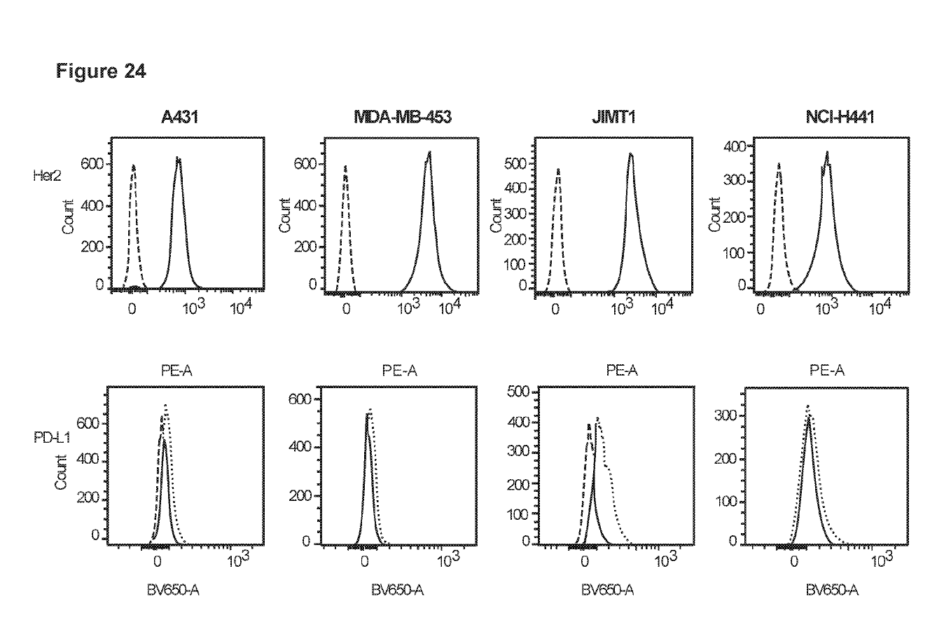

To identify relevant cell lines in which to assess the biological activity of exemplary Her2 targeting MIACs, we screened a panel of cancer cell lines for Her2 and PD-L1 (programmed death ligand-1) surface expression by flow cytometry. The results in FIG. 24 show that the JIMT1 cell line has high Her2 expression and expressed the immunoinhibitory molecule, programmed death ligand-1 (PD-L1; CD274) on its surface. Based on these findings, JIMT1 cells were selected for in vitro functional assessment of the exemplary Her2-targeting MIACs.

### Example 19: Binding Analysis of Exemplary Her2-Targeting MIACs to Tumor Cells by Flow Cytometry

The binding of exemplary MIAC constructs to the surface of Her2+ JIMT1 cells was analyzed by flow cytometry. The experiment was carried out as follows.

JIMT1 cells in suspension were incubated in the presence of increasing concentrations (0, 0.01, 0.1, 1.0 nM) of bispecific and trispecific Her2-targeting MIACs (PID92 [Her2/CD137], PID128 [Her2/CD3], PID130 [Her2/CD3/PD-1]) or control antibodies against Her2 (positive control) or CD3 (negative control) for 30 minutes. Cells were washed and then incubated with a secondary fluorophore-labeled detection antibody (anti-human IgG1-AF488) for 20 minutes. After a final wash, binding of the proteins, by mean fluorescence intensity (MFI) was analyzed using a BD Fortessa flow cytometer.

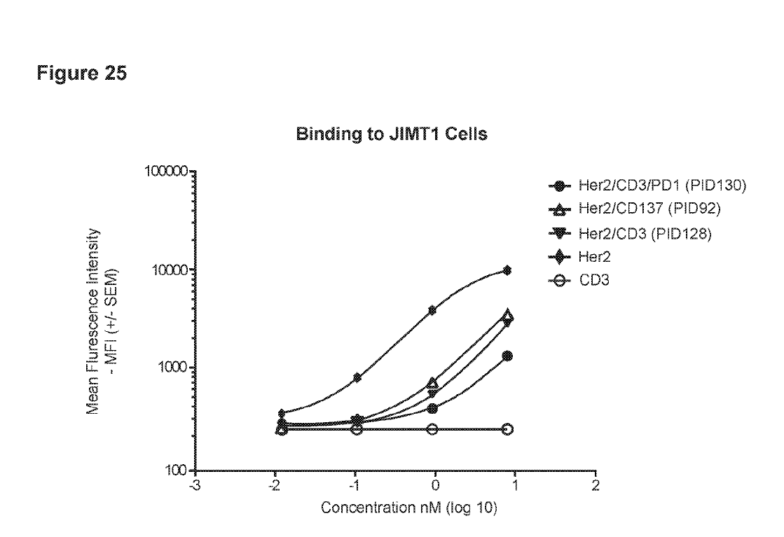

As shown in FIG. 25, all three of the exemplary MIACs displayed detectable, concentration-dependent binding to JIMT1 cells. The level of MIAC binding was lower than that observed with the anti-Her2 positive control antibody, suggesting that the linkage of the Her2 binding scFvs to the C-termini of the antibody heavy chains can have led to a reduction in Her2 antigen binding affinity/avidity. The level of cell binding activity exhibited by the MIACs is considered sufficient to assess biological activity in vitro.

### Example 20: Effects of an Exemplary Bispecific Anti-Her2/Anti-CD137 MIAC (PID92) on Proliferation and CD25 Activation Marker Expression in Human T Cell/JIMT1 Tumor Cell Co-Cultures

This experiment was performed to determine the ability of an exemplary bispecific MIAC1, targeting Her2 and CD137, to enhance the proliferation and activation of human primary T cells co-cultured with JIMT1 tumor cells. The response of the α-Her2/α-CD137 MIAC (PID92) was compared against equimolar concentrations of combined monospecific α-Her2 and α-CD137 antibodies possessing identical Fab sequences as the MIAC. The experiment was carried out as follows.

CD3+ T cells were isolated by Ficoll gradient centrifugation followed by magnetic bead separation (Miltenyi Biotec). Prior to being placed in co-culture with JIMT1 breast carcinoma cells at a ratio of 10:1, T cells were labeled with carboxyfluorescein succinimidyl ester (CFSE) dye. Co-cultures were seeded in 96-well microplates pre-coated with 1 μg/ml of anti-CD3 antibody (clone OKT3) followed by incubation for 72 hours in the presence of increasing concentrations (0.02 to 20.0 nM) of bispecific α-Her2/α-CD137 MIAC (PID92) or an equimolar concentration of combined α-Her2 and α-CD137 monoclonal antibodies. At the end of the incubation period, CD4+ and CD8+ T cells were analyzed for proliferation and CD25 expression by flow cytometry following staining with fluorophore-labeled antibodies against CD4, CD8, and CD25. Percent proliferation was defined as the proportion of cells experiencing at least one round of cell division as determined by CFSE signal dilution.

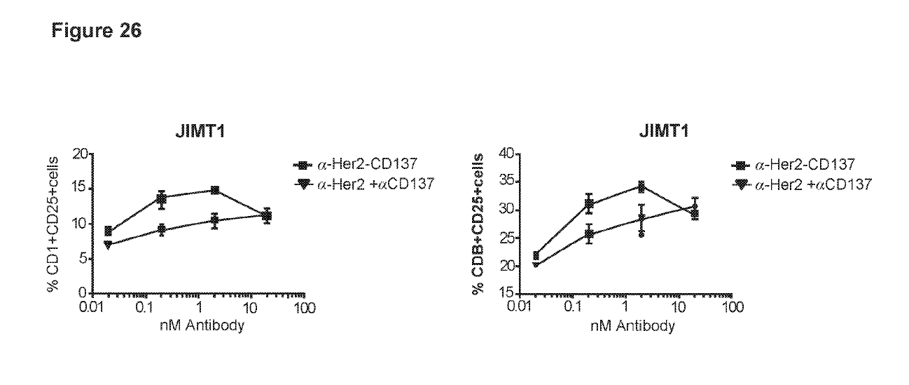

Results of CD25 expression analysis are shown in FIG. 26. Compared to the combination of monospecific α-Her2 and α-CD137 antibodies, the bispecific MIAC stimulated higher surface expression of CD25 in both CD4+ and CD8+ T cell subsets. The enhanced effect of the α-Her2/α-CD137 MIAC (PID92) on CD25 expression was evident across the tested concentration range, with the exception of the lowest and highest concentrations tested, which showed similar levels of expression for both the MIAC and the combination treatment.

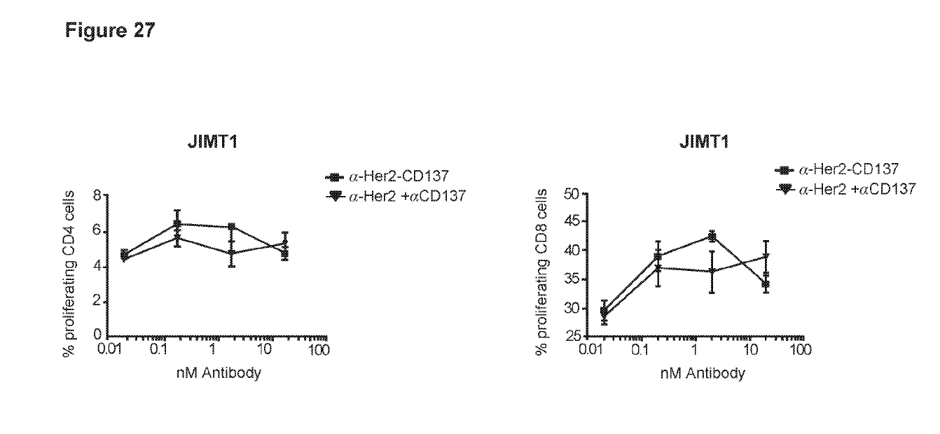

Results of the proliferation analysis are shown in FIG. 27. Concentration-dependent increases in proliferation were more pronounced in CD8+ vs. CD4+ T cells. Although the overall magnitude of proliferation induced by the α-Her2/α-CD137 MIAC (PID92) was similar to the antibody combination, there was a pronounced separation of the response curves at 2 nM, suggesting enhanced potency of the MIAC.

### Example 21: Effects of an Exemplary Bispecific Anti-Her2/Anti-CD137 MIAC (PID92) on Cytokine Production in Human T Cell/JIMT1 Tumor Cell Co-Cultures

This experiment was performed to determine the ability of an exemplary bispecific MIAC, targeting Her2 and CD137, to enhance cytokine production from human primary T cells co-cultured with JIMT1 tumor cells. As a control, the effects of the α-Her2/α-CD137 MIAC (PID92) MIAC were compared against equimolar concentrations of combined mono-specific α-Her2 and α-CD137 antibodies possessing the same Fab sequences as the MIAC.

Cytokine production was determined in cell culture supernatants collected from the same co-culture experiment described in Example 6. At the end of the 72 hour incubation period, supernatants were collected and levels of IFN-γ and TNF-α were measured using a Luminex kit (EMD Millipore).

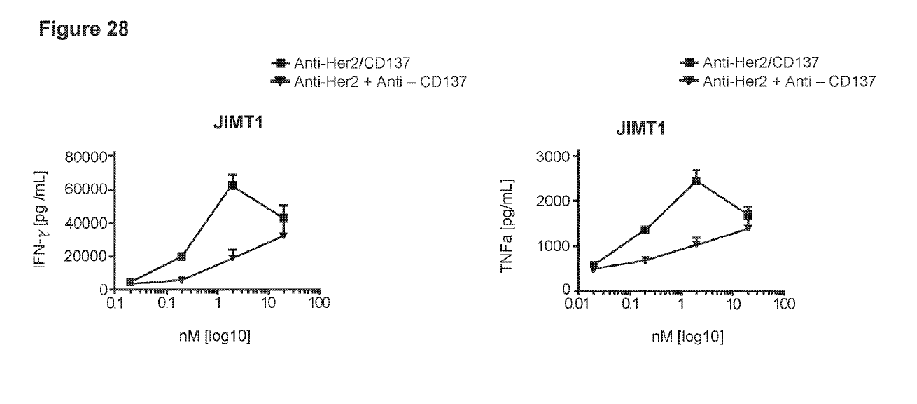

Results are shown in FIG. 28. Robust, concentration-dependent increases in both IFN-γ and TNF-α were observed, with the bispecific α-Her2/α-CD137 MIAC (PID92) demonstrating dramatically enhanced activity compared to the monospecific antibody combination.

In addition, a similar experiment was run to measure IL-2 production from T cells in co-culture with Her2+ SKBR3 cells. It was found that PID92 at 100 nM increased IL-2 production by approximately 4-fold relative to an equimolar concentration of PID90.

### Example 22: Effects of an Exemplary Bispecific Anti-Her2/Anti-CD3 MIAC (PID128) on Proliferation and CD25 Activation Marker Expression in Human T Cell/JIMT1 Tumor Cell Co-Cultures

This experiment was performed to determine the ability of an exemplary bispecific MIAC, targeting Her2 and CD3, to enhance the proliferation and activation of human primary T cells co-cultured with JIMT1 tumor cells. As a control, the effects of the a-Her2/α-CD3 MIAC (PID128) MIAC were compared against equimolar concentrations of a CD3 targeted antibody possessing the same Fab sequence contained in the MIAC. The experiment was carried out as follows.

CD3+ T cells were isolated by Ficoll gradient centrifugation followed by magnetic bead separation (Miltenyi Biotec) and placed in co-culture with JIMT1 breast carcinoma cells at a ratio of 10:1. Co-cultures were seeded in 96-well microplates followed by incubation for 72 hours in the presence of increasing concentrations (0.02 to 20.0 nM) of the bispecific α-Her2/α-CD3 MIAC (PID128) MIAC or equimolar concentrations of anti-CD3 monoclonal antibody. At the end of the incubation period, T cells were analyzed by flow cytometry for proliferation and CD25 surface expression following staining with fluorophore-labeled antibodies against Ki67 (an intracellular proliferation marker), CD8, and CD25.

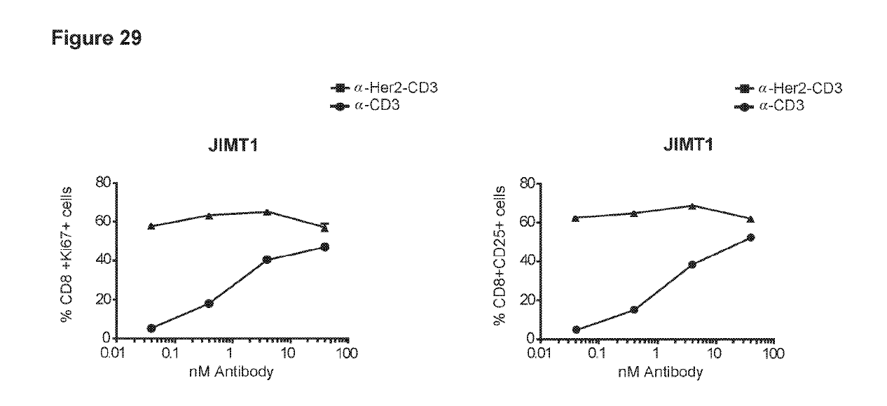

Results of the proliferation and CD25 expression analysis are shown in FIG. 29. The bispecific α-Her2/α-CD3 MIAC (PID128) MIAC dramatically upregulated both T cell proliferation and surface expression of CD25, with maximal induction observed at all tested concentrations. In comparison, the effects of the anti-CD3 antibody were much less potent and concentration-dependent.

### Example 23: Effects of an Exemplary Bispecific Anti-Her2/Anti-CD3 MIAC (PID128) on Cytokine and Granzyme B Production in Human T Cell/JIMT1 Tumor Cell Co-Cultures

This experiment was performed to determine the ability of an exemplary bispecific MIAC, targeting Her2 and CD3, to enhance cytokine production from human primary T cells co-cultured with JIMT1 tumor cells. As a control, the α-Her2/α-CD3 MIAC (PID128) MIAC was compared against equimolar concentrations of a mono-specific anti-CD3 antibody possessing the same Fab sequences as the MIAC.

Cytokine production was determined in cell culture supernatants collected from the same co-culture experiment described in Example 8. At the end of the 72 hour incubation period, supernatants were collected and levels of IFN-γ, TNF-α, and granzyme B were measured using a Luminex kit (EMD Millipore).

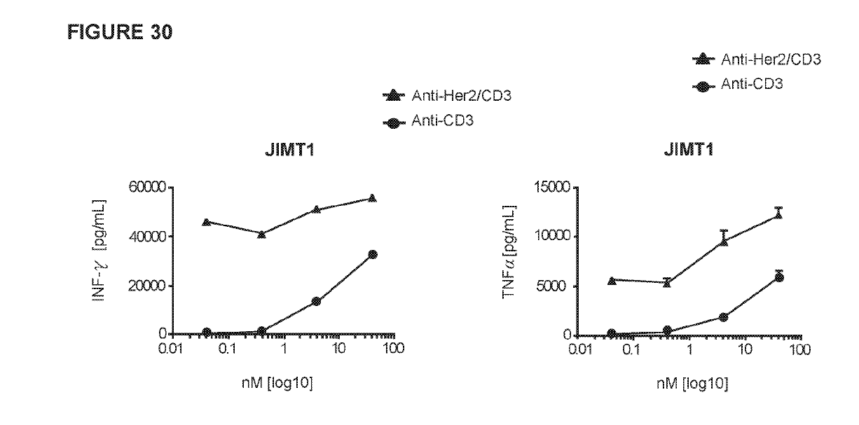

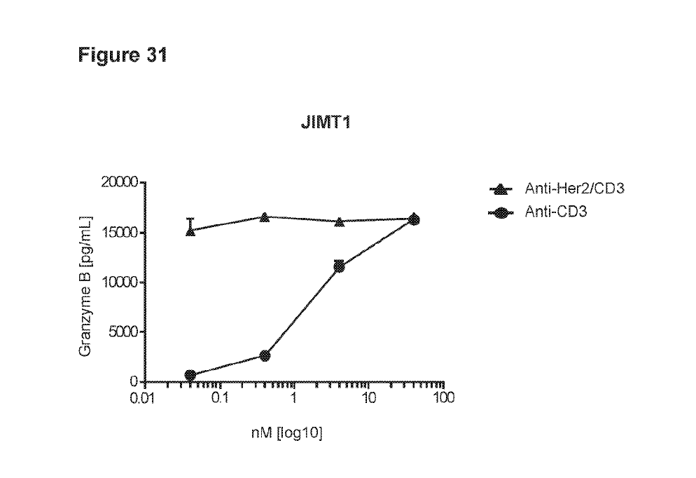

Robust, concentration-dependent increases in both IFN-γ and TNF-α were observed (FIG. 30), with the bispecific α-Her2/α-CD3 MIAC (PID128) MIAC demonstrating dramatically enhanced potency compared to the monospecific CD3 antibody.

Granzyme B, a protease found in the cytotoxic granules of CD8+ T cells that mediates killing of target cells, was also strongly induced by treatment with the α-Her2/α-CD3 MIAC (PID128) MIAC (FIG. 31) and showed dramatically increased potency compared to monospecific anti-CD3. The MIAC achieved maximal production of granzyme B across the entire concentration range tested, including the lowest concentration of 0.02 nM.

### Example 24: Effects of an Exemplary Trispecific Anti-Her2/Anti-CD3/Anti-PD-1 MIAC (PID130) on Proliferation and CD25 Activation Marker Expression in Human T Cell/JIMT1 Tumor Cell Co-Cultures

This experiment was performed to determine the ability of an exemplary trispecific MIAC, targeting Her2, CD3, and PD-1 (programmed death receptor-1) to enhance the proliferation and activation of human primary T cells co-cultured with JIMT1 tumor cells. As a control, the effects of the MIAC were compared against equimolar concentrations of combined monospecific anti-Her2, anti-CD3, and anti-PD-1 antibodies possessing identical Fab sequences as the trispecific α-Her2/α-CD3/α-PD-1 MIAC (PID130). The experiment was carried out as follows.

CD3+ T cells were isolated by Ficoll gradient centrifugation followed by magnetic bead separation (Miltenyi Biotec) and placed in co-culture with JIMT1 breast carcinoma cells at a ratio of 10:1. Co-cultures were seeded in 96-well microplates followed by incubation for 72 hours in the presence of increasing concentrations (0.02 to 20.0 nM) of the α-Her2/α-CD3/α-PD-1 MIAC (PID130) or equimolar concentrations of combined monospecific anti-Her2, anti-CD3, and anti-PD-1 monoclonal antibodies. At the end of the incubation period, T cells were analyzed by flow cytometry for proliferation and CD25 surface expression following staining with fluorophore-labeled antibodies against Ki67 (an intracellular proliferation marker), CD8, and CD25.

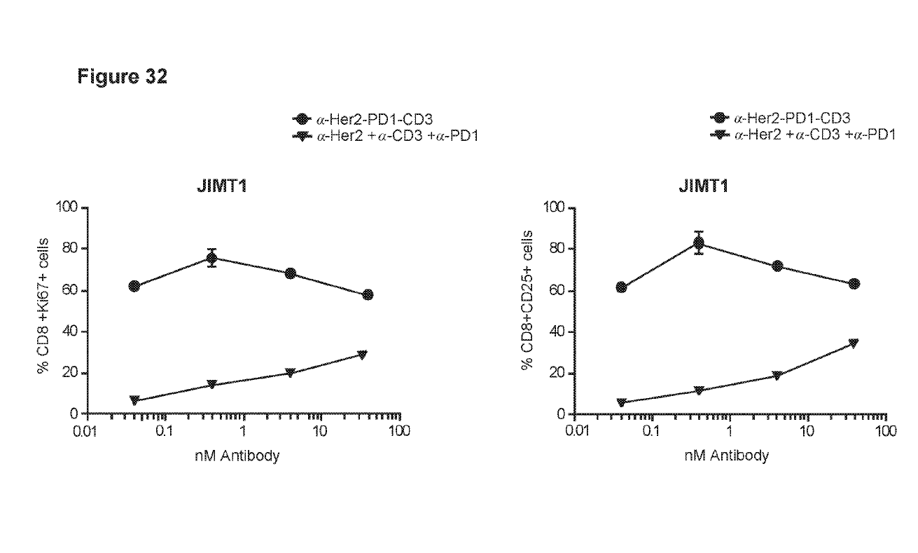

Results of the proliferation and CD25 expression analysis are shown in FIG. 32. The trispecific α-Her2/α-CD3/α-PD-1 MIAC (PID130) dramatically upregulated both T cell proliferation and surface expression of CD25, with near maximal induction observed across all tested concentrations. In comparison, the triple combination of monospecific antibodies showed concentration-dependent responses that were far less potent than the MIAC.

### Example 25: Effects of an Exemplary Trispecific Anti-Her2/Anti-CD3/Anti-PD-1 MIAC (PID130) on Cytokine and Granzyme B Production in Human T Cell/JIMT1 Tumor Cell Co-Cultures

This experiment was performed to determine the ability of an exemplary trispecific MIAC, targeting Her2, CD3, and PD-1, to enhance cytokine production from human primary T cells co-cultured with JIMT1 tumor cells. As a control, the α-Her2/α-CD3/α-PD-1 MIAC (PID130) was compared against equimolar concentrations of combined monospecific antibodies against Her2, CD3 and PD-1 that possessed the identical Fab sequences as the MIAC.

Cytokine production was determined in cell culture supernatants collected from the same co-culture experiment described in Example 10. At the end of the 72 hour incubation period, supernatants were collected and levels of IFN-γ, TNF-α, and granzyme B were measured using a Luminex kit (EMD Millipore).

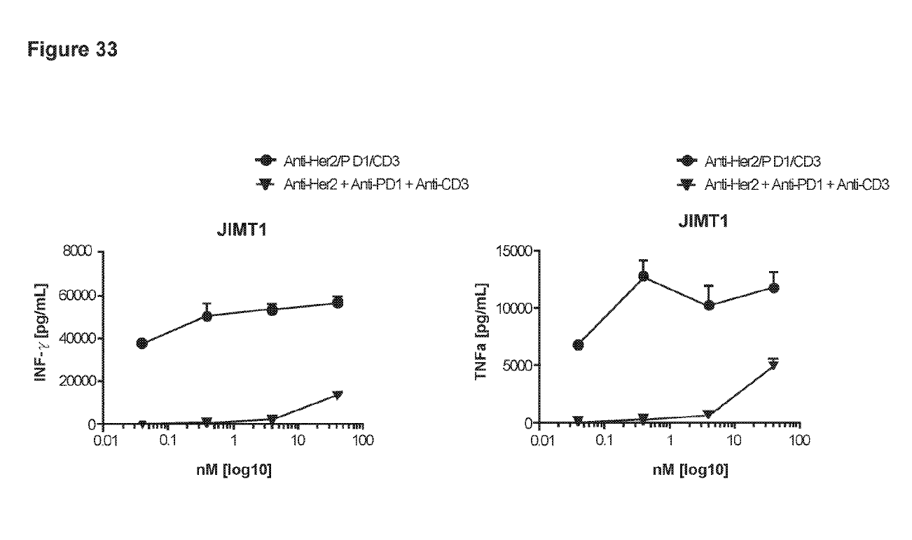

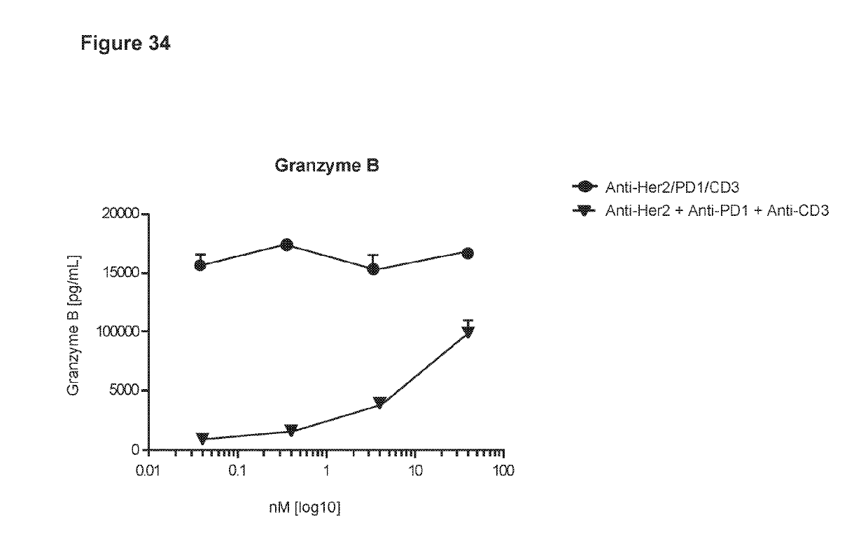

As shown in FIG. 33, the trispecific α-Her2/α-CD3/α-PD-1 MIAC (PID130) dramatically increased both IFN-γ and TNF-α production with a near maximal effect observed across the entire concentration range tested. In contrast, the combined effect of monospecific antibodies was far less potent and concentration-dependent, with increases in cytokine production only being observed at the high end of the concentration response curve. Granzyme B, a protease found in the cytotoxic granules of CD8+ T cells that mediates killing of target cells, was also induced at high levels by treatment with the α-Her2/α-CD3/α-PD-1 MIAC (PID130) (FIG. 34) and, as with cytokine production, maximal effects were seen at even the lowest tested concentration. In comparison, the combination of monospecific antibodies did increase granzyme B in a concentration-dependent manner, but demonstrated much lower potency than the trispecific MIAC.

### Example 26: Effects of an Exemplary Bispecific Anti-Her2/Anti-CD3 MIAC (PID128) on T Cell-Mediated Tumor Killing, Granzyme B Induction, and IFN-γ Production in Human T Cell/BT474 Tumor Cell Co-Cultures

This experiment was performed to determine the ability of an exemplary bispecific MIAC, targeting Her2 and CD3, to enhance the production of gamma interferon and promote the killing of the human BT474 breast cell line. The response of the α-Her2/α-CD3 MIAC (PID128) was compared against equimolar concentrations of combined monospecific α-Her2 and α-CD3 antibodies possessing identical Fab sequences as the MIAC. The experiment was carried out as follows.

CD3+ T cells were isolated by Ficoll gradient centrifugation followed by magnetic bead separation (Miltenyi Biotec) and co-cultured the BT474 breast carcinoma cells at a ratio of 15:1 in a 96-well culture plate. Prior to cell plating, increasing concentrations (0.01 nM to 1.0 nM) of bispecific α-Her2/α-CD3 MIAC (PID128) or combined α-Her2 and α-CD3 monoclonal antibodies were added to appropriate wells in the plate. The plates were incubated for 24 hours and the supernatants were collected and used for the CytoTox 96® Non-Radioactive Cytotoxicity and Luminex cytokine assays according to the manufacturers' protocols.

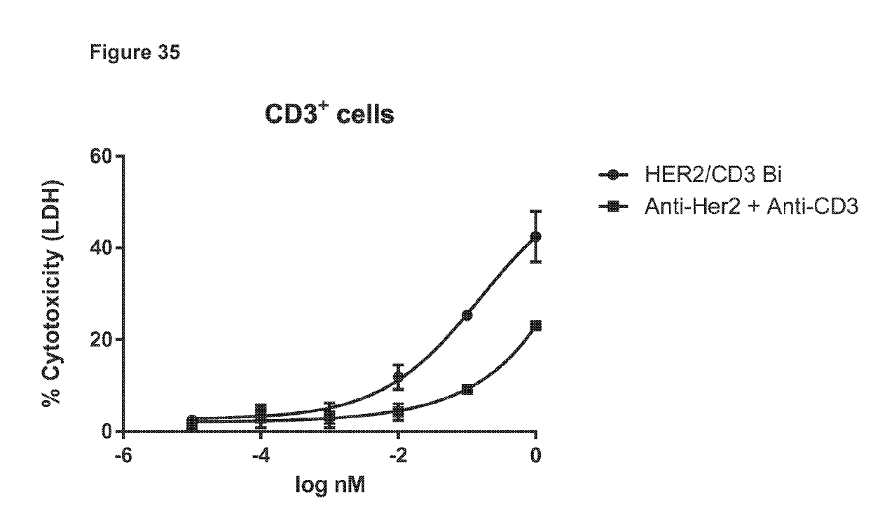

Results of tumor cell killing analysis are shown in FIG. 35. Compared to the combination of monospecific α-Her2 and α-CD3 antibodies, the bispecific MIAC mediated more cell killing than the combination of the two monoclonal antibodies.

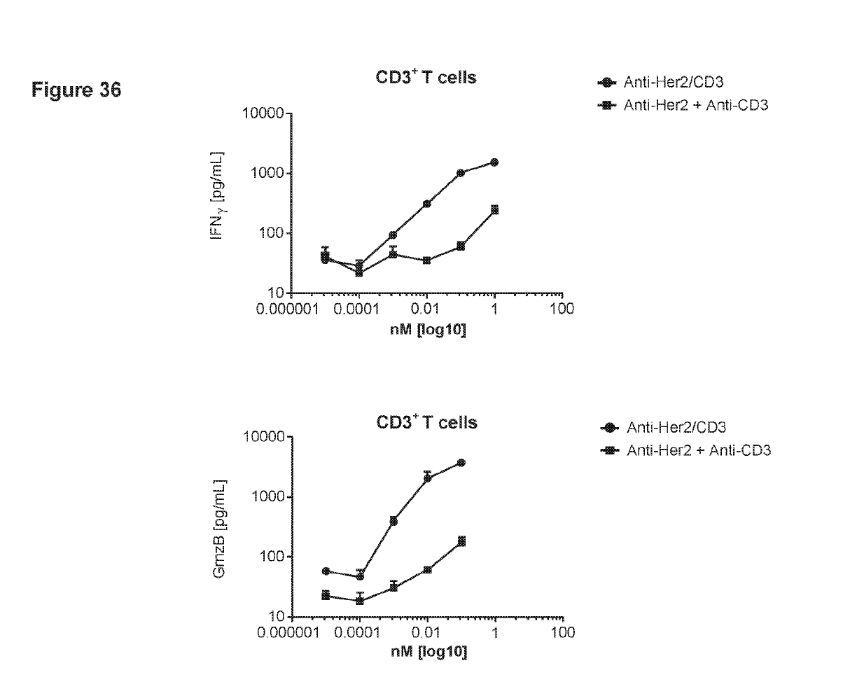

Results of the IFN-γ and granzyme B production analysis are shown in FIG. 36. Concentration-dependent increases in production of IFN-γ and granzyme B were significantly more pronounced in the presence of the α-Her2/α-CD3 MIAC (PID128) compared to the combination of the monoclonal antibodies. Together, these results show that the α-Her2/α-CD3 MIAC (PID218) is more potent at inducing cytokine production and promoting tumor cell killing than the combination of the monoclonal antibodies.

### Other Embodiments, Incorporation by Reference

The disclosure set forth herein can encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties can be claimed in this application, in applications claiming priority from this application, or in related applications. Such claims, whether directed to a different invention or to the same invention, and whether broader, narrower, equal, or different in scope in comparison to the original claims, also are included within the subject matter of the inventions of the present disclosure.

All references, publications, and patents cited in this disclosure are hereby incorporated by reference in their entireties.

SEQUENCES

SEQ ID NO: 1-Heavy Chain #1 from Example 13
Brentuximab VH_human IgG1 HC constant (GGGGS)3_Urelumab
VH_(GGGGS)7_Urelumab_VK (“(GGGGS)3” and “(GGGGS)7” disclosed as SEQ ID NOS
23 and 97, respectively)
QIQLQQSGPEVVKPGASVKISCKASGYTFTDYYITWVKQKPGQGLEWIGWIYPGSGNTKYNEKFKGKATLTVDT
SSSTAFMQLSSLTSEDTAVYFCANYGNYWFAYWGQGTQVTVSAASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDK
THTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEALHNHYTQKSLSLS
PGKGGGGSGGGGSGGGGSQVQLQQWGAGLLKPSETLSLTCAVYGGSFSGYYWSWIRQSPEKGLEWIGEINHGGY
VTYNPSLESRVTISVDTSKNQFSLKLSSVTAADTAVYYCARDYGPGNYDWYFDLWGRGTLVTVSSGGGGSGGGG
SGGGGSGGGGSGGGGSGGGGSGGGGSEIVLTQSPATLSLSPGERATLSCRASQSVSSYLAWYQQKPGQAPRLLI
YDASNRATGIPARFSGSGSGTDFTLTISSLEPEDFAVYYCQQRSNWPPALTFCGGTKVEIK
SEQ ID NO: 2-Heavy Chain #2 from Example 13
Brentuximab VH_human IgG1 constant_(GGGGS)3_Pembrolizumab
VH_(GGGGS)7_Pembrolizumab_VK_(“(GGGGS)3” and “(GGGGS)7” disclosed as
SEQ ID NOS 23 and 97, respectively)
QIQLQQSGPEVVKPGASVKISCKASGYTFTDYYITWVKQKPGQGLEWIGWIYPGSGNTKYNEKFKGKATLTVDT
SSSTAFMQLSSLTSEDTAVYFCANYGNYWFAYWGQGTQVTVSAASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDK
THTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEALHNHYTQKSLSLS
PGKGGGGSGGGGSGGGGSQVQLVQSGVEVKKPGASVKVSCKASGYTFTNYYMYWVRQAPGQGLEWMGGINPSNG
GTNFNEKFKNRVTLTTDSSTTTAYMELKSLQFDDTAVYYCARRDYRFDMGFDYWGQGTTVTVSSGGGGSGGGGS
GGGGSGGGGSGGGGSGGGGSGGGGSEIVLTQSPATLSLSPGERATLSCRASKGVSTSGYSYLHWYQQKPGQAPR
LLIYLASYLESGVPARFSGSGSGTDFTLTISSLEPEDFAVYYCQHSRDLPLTFGGGTKVEIK
SEQ ID NO: 3-Light Chain from Example 13
Brentuximab VL_human IgG1 LC constant
DIVLTQSPASLAVSLGQRATISCKASQSVDFDGDSYMNWYQQKPGQPPKVLIYAASNLESGIPARFSGSGSGTD
FTLNIHPVEEEDAATYYCQQSNEDPWTFGGGTKLEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKSFNRGEC
SEQ ID NO: 5-Heavy Chain from Example 14
Pembrolizumab VH_(GGGGS)2_Urelumab VH_human IgG1 HC
constant_(GGGGS)3_Brentuximab VH_(GGGGS)7_Brentuximab_VK (“(GGGGS)2,”
“(GGGGS)3” and “(GGGGS)7” disclosed as SEQ ID NOS 26, 23 and 97,
respectively)
QVQLVQSGVEVKKPGASVKVSCKASGYTFTNYYMYWVRQAPGQGLEWMGGINPSNGGTNFNEKFKNRVTLTTDS
STTTAYMELKSLQFDDTAVYYCARRDYRFDMGFDYWGQGTTVTVSSGGGGSGGGGSQVQLQQWGAGLLKPSETL
SLTCAVYGGSFSGYYWSWIRQSPEKGLEWIGEINHGGYVTYNPSLESRVTISVDTSKNQFSLKLSSVTAADTAV
YYCARDYGPGNYDWYFDLWGRGTLVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKKVEPKSCDKTHTCPPCPAPELLG
GPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPGKGGGGSGGGGSG
GGGSQIQLQQSGPEVVKPGASVKISCKASGYTFTDYYITWVKQKPGQGLEWIGWIYPGSGNTKYNEKFKGKATL
TVDTSSSTAFMQLSSLTSEDTAVYFCANYGNYWFAYWGQGTQVTVSAGGGGSGGGGSGGGGSGGGGSGGGGSGG
GGSGGGGSDIVLTQSPASLAVSLGQRATISCKASQSVDFDGDSYMNWYQQKPGQPPKVLIYAASNLESGIPARF
SGSGSGTDFTLNIHPVEEEDAATYYCQQSNEDPWTFGGGTKLEIK
SEQ ID NO: 6-Light Chain from Example 14
Pembrolizumab VL_(GGGGS)2_Urelumab VL_human IgG1 LC constant (“(GGGGS)2”
disclosed as SEQ ID NO: 26)
EIVLTQSPATLSLSPGERATLSCRASKGVSTSGYSYLHWYQQKPGQAPRLLIYLASYLESGVPARFSGSGSGTD
FTLTISSLEPEDFAVYYCQHSRDLPLTFGGGTKVEIKGGGGSGGGGSEIVLTQSPATLSLSPGERATLSCRASQ
SVSSYLAWYQQKPGQAPRLLIYDASNRATGIPARFSGSGSGTDFTLTISSLEPEDFAVYYCQQRSNWPPALTFC
GGTKVEIKRTVAAPSVFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
SEQ ID NO: 7-Heavy Chain (DID-I)
Trastuzumab VH_human IgG1 HC constant
EVQLVESGGGLVQPGGSLRLSCAASGFNIKDTYIHWVRQAPGKGLEWVARTYPTNGYTRYADSVKGRFTISADT
SKNTAYLQMNSLRAEDTAVYYCSRWGGDGFYAMDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVMHEALHNHYTQKSL
SLSPGK
SEQ ID NO: 8-Light Chain (DID-2)
Trastuzumab VL_human Kappa LC constant
DIQMTQSPSSLSASVGDRVTITCRASQDVNTAVAWYQQKPGKAPKLLIYSASFLYSGVPSRFSGSRSGTDFTLT
ISSLQPEDFATYYCQQHYTTPPTFGQGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
Trastuzumab heavy chain CDRs
CDR-H1: GFNIKDT (SEQ ID NO: 28)
CDR-H2: YPTNGY (SEQ ID NO: 29)
CDR-H3: WGGDGFYAMDY (SEQ ID NO: 30)
Trastuzumab light chain CDRs
CDR-L1: RASQDVNTAVA (SEQ ID NO: 31)
CDR-L2: SASFLYS (SEQ ID NO: 32)
CDR-L3: QQHYTTPPT (SEQ ID NO: 33)
SEQ ID NO: 9-Heavy Chain (DID-365)
Blinatumomab_CD3_VH_human aglycosylated IgG1 constant
DIKLQQSGAELARPGASVKMSCKTSGYTFTRYTMHWVKQRPGQGLEWIGYINPSRGYTNYNQKFKDKATLITDK
SSSTAYMQLSSLTSEDSAVYYCARYYDDHYCLDYWGQGTTLTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKFNWYVDGVEVHNAKTKPREE
QYASTYRVVSVLTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHEALHNHYTQKSLS
LSPGK
SEQ ID NO: 10-Light Chain (DID-8)
Blinatumomab_CD3 VL_human Kappa LC constant
DIQLTQSPAIMSASPGEKVTMTCRASSSVSYMNWYQQKSGTSPKRWIYDTSKVASGVPYRFSGSGSGTSYSLTI
SSMEAEDAATYYCQQWSSNPLTFGAGTKLELKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC
Blinatumomab heavy chain CDRs
CDR-H1: GYTFTRYTMH (SEQ ID NO: 34)
CDR-H2: YINPSRGYT (SEQ ID NO: 35)
CDR-H3: YYDDHYCLDY (SEQ ID NO: 36)
Blinatumomab light chain CDRs
CDR-L1: RASSSVSYMN (SEQ ID NO: 37)
CDR-L2: DTSKVAS (SEQ ID NO: 38)
CDR-L3: QQWSSNPLT (SEQ ID NO: 39)
SEQ ID NO: 11-Heavy Chain (DID-9)
Urelumab VH_human IgG1 constant
QVQLQQWGAGLLKPSETLSLTCAVYGGSFSGYYWSWIRQSPEKGLEWIGEINHGGYVTYNPSLESRVTISVDTS
KNQFSLKLSSVTAADTAVYYCARDYGPGNYDWYFDLWGRGTLVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQKS
LSLSPGK
SEQ ID NO: 12-Light Chain (DID-309)
Urelumab VL_human Kappa LC constant
EIVLTQSPATLSLSPGERATLSCRASQSVSSYLAWYQQKPGQAPRLLIYDASNRATGIPARFSGSGSGTDFTLT
ISSLEPEDFAVYYCQQRSNWPPALTFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
Urelumab heavy chain CDRs
CDR-H1: GGSFSGYY (SEQ ID NO: 40)
CDR-H2: NHGGY (SEQ ID NO: 41)
CDR-H3: DYGPGNYDWYFDL (SEQ ID NO: 42)
Urelumab light chain CDRs
CDR-L1: RASQSVSSYLA (SEQ ID NO: 43)
CDR-L2: DASNRAT (SEQ ID NO: 44)
CDR-L3: QQRSNWPPALT (SEQ ID NO: 45)
SEQ ID NO: 13-Heavy Chain (DID-15)
Pembrolizumab VH_human IgG1 constant
QVQLVQSGVEVKKPGASVKVSCKASGYTFTNYYMYWVRQAPGQGLEWMGGINPSNGGTNFNEKFKNRVTLTTDS
STTTAYMELKSLQFDDTAVYYCARRDYRFDMGFDYWGQGTTVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVMHEALHNHYTQKSL
SLSPGK
SEQ ID NO: 14-Light Chain (DID-16)
Pembrolizumab VL_human Kappa LC constant
EIVLTQSPATLSLSPGERATLSCRASKGVSTSGYSYLHWYQQKPGQAPRLLIYLASYLESGVPARFSGSGSGTD
FTLTISSLEPEDFAVYYCQHSRDLPLTFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKSFNRGEC
Pembrolizumab heavy chain CDRs
CDR-H1: GYTFTNYYMY (SEQ ID NO: 46)
CDR-H2: NPSNGG (SEQ ID NO: 47)
CDR-H3: RDYRFDMGFDY (SEQ ID NO: 48)
Pembrolizumab light chain CDRs
CDR-L1: RASKGVSTSGYSYLH (SEQ ID NO: 49)
CDR-L2: LASYLES (SEQ ID NO: 50)
CDR-L3: QHSRDLPLT (SEQ ID NO: 51)
SEQ ID NO: 15-Heavy Chain (DID-366)
Blinatumomab_CD3 VH_human aglycosylated IgG1 HC constant_(GGGGS)3
Trastuzumab VH_(GGGGS)4_Trastuzumab VL(“(GGGGS)3” and “(GGGGS)4”
disclosed as SEQ ID NOS 23 and 24, respectively)
DIKLQQSGAELARPGASVKMSCKTSGYTFTRYTMEIWVKQRPGQGLEWIGYINPSRGYTNYNQKFKDKATLITD
KSSSTAYMQLSSLTSEDSAVYYCARYYDDHYCLDYWGQGTTLTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVMHEALHNHYTQKSL
SLSPGKGGGGSGGGGSGGGGSEVQLVESGGGLVQPGGSLRLSCAASGFNIKDTYIHWVRQAPGKGLEWVARIYP
TNGYTRYADSVKGRFTISADTSKNTAYLQMNSLRAEDTAVYYCSRWGGDGFYAMDYWGQGTLVTVSSGGGGSGG
GGSGGGGSGGGGSDIQMTQSPSSLSASVGDRVTITCRASQDVNTAVAWYQQKPGKAPKLLIYSASFLYSGVPSR
FSGSRSGTDFTLTISSLQPEDFATYYCQQHYTTPPTFGQGTKVEIK
Blinatumomab heavy chain CDRs
CDR-H1: GYTFTRYTMH (SEQ ID NO: 52)
CDR-H2: YINPSRGYT (SEQ ID NO: 53)
CDR-H3: YYDDHYCLDY (SEQ ID NO: 54)
Trastuzumab heavy chain CDRs
CDR-H1: GFNIKDT (SEQ ID NO: 55)
CDR-H2: YPTNGY (SEQ ID NO: 56)
CDR-H3: WGGDGFYAMD (SEQ ID NO: 57)
Trastuzumab light chain CDRs
CDR-L1: RASQDVNTAVA (SEQ ID NO: 58)
CDR-L2: SASFLYS (SEQ ID NO: 59)
CDR-L3: QQHYTTPPT (SEQ ID NO: 60)
SEQ ID NO: 16-Heavy Chain (DID-26)
Urelumab VH_human IgG1 HC constant_(GGGGS)3_Trastuzumab
VH_(GGGGS)4_Trastuzumab VL(“(GGGGS)3” and “(GGGGS)4” disclosed as
SEQ ID NOS 23 and 24, respectively)
QVQLQQWGAGLLKPSETLSLTCAVYGGSFSGYYWSWIRQSPEKGLEWIGEINHGGYVTYNPSLESRVTISVDTS
KNQFSLKLSSVTAADTAVYYCARDYGPGNYDWYFDLWGRGTLVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQKS
LSLSPGKGGGGSGGGGSGGGGSEVQLVESGGGLVQPGGSLRLSCAASGFNIKDTYIHWVRQAPGKGLEWVARTY
PTNGYTRYADSVKGRFTISADTSKNTAYLQMNSLRAEDTAVYYCSRWGGDGFYAMDYWGQGTLVTVSSGGGGSG
GGGSGGGGSGGGGSDIQMTQSPSSLSASVGDRVTITCRASQDVNTAVAWYQQKPGKAPKWYSASFLYSGVPSRF
SGSRSGTDFTLTISSLQPEDFATYYCQQHYTTPPTFGQGTKVEIK
Urelumab heavy chain CDRs
CDR-H1: GGSFSGYY (SEQ ID NO: 61)
CDR-H2: NHGGY (SEQ ID NO: 62)
CDR-H3: DYGPGNYDWYFDL (SEQ ID NO: 63)
Trastuzumab heavy chain CDRs
CDR-H1: GFNIKDT (SEQ ID NO: 64)
CDR-H2: YPTNGY (SEQ ID NO: 65)
CDR-H3: WGGDGFYAMDY (SEQ ID NO: 66)
Trastuzumab light chain CDRs
CDR-L1: RASQDVNTAVA (SEQ ID NO: 67)
CDR-L2: SASFLYS (SEQ ID NO: 68)
CDR-L3: QQHYTTPPT (SEQ ID NO: 69)
SEQ ID NO: 17-Heavy Chain (DID-27)
Pembrolizumab VH_human IgG1 HC constant_(GGGGS)3_Trastuzumab
VH_(GGGGS)4_Trastuzumab VL(“(GGGGS)3” and “(GGGGS)4” disclosed as
SEQ ID NOS 23 and 24, respectively)
QVQLVQSGVEVKKPGASVKVSCKASGYTFTNYYMYWVRQAPGQGLEWMGGINPSNGGTNFNEKFKNRVTLTTDS
STTTAYMELKSLQFDDTAVYYCARRDYRFDMGFDYWGQGTTVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVMHEALHNHYTQKSL
SLSPGKGGGGSGGGGSGGGGSEVQLVESGGGLVQPGGSLRLSCAASGFNIKDTYIHWVRQAPGKGLEWVARTYP
TNGYTRYADSVKGRFTISADTSKNTAYLQMNSLRAEDTAVYYCSRWGGDGFYAMDYWGQGTLVTVSSGGGGSGG
GGSGGGGSGGGGSDIQMTQSPSSLSASVGDRVTITCRASQDVNTAVAWYQQKPGKAPKWYSASFLYSGVPSRFS
GSRSGTDFTLTISSLQPEDFATYYCQQHYTTPPTFGQGTKVEIK
Pembrolizumab heavy chain CDRs
CDR-H1: GYTFTNYYMY (SEQ ID NO: 70)
CDR-H2: NPSNGG (SEQ ID NO: 71)
CDR-H3: RDYRFDMGFDY (SEQ ID NO: 72)
Trastuzumab heavy chain CDRs
CDR-H1: GFNIKDT (SEQ ID NO: 73)
CDR-H2: YPTNGY (SEQ ID NO: 74)
CDR-H3: WGGDGFYAMDY (SEQ ID NO: 75)
Trastuzumab light chain CDRs
CDR-L1: RASQDVNTAVA (SEQ ID NO: 76)
CDR-L2: SASFLYS (SEQ ID NO: 77)
CDR-L3: QQHYTTPPT (SEQ ID NO: 78)
SEQ ID NO: 18-Light Chain (DID-50)
Blinatumomab_CD3 VL_human Kappa LC constant_(GGGGS)3_Pembrolizumab
VH_(GGGGS)4_Pembrolizumab VL(“(GGGGS)3” and “(GGGGS)4” disclosed as
SEQ ID NOS 23 and 24, respectively)
DIQLTQSPAIMSASPGEKVTMTCRASSSVSYMNWYQQKSGTSPKRWIYDTSKVASGVPYRFSGSGSGTSYSLTI
SSMEAEDAATYYCQQWSSNPLTFGAGTKLELKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGECGGGGSGGGG
SGGGGSQVQLVQSGVEVKKPGASVKVSCKASGYTFTNYYMYWVRQAPGQGLEWMGGINPSNGGTNFNEKFKNRV
TLITDSSTITAYMELKSLQFDDTAVYYCARRDYRFDMGFDYWGQGTTVTVSSGGGGSGGGGSGGGGSGGGGSEI
VLTQSPATLSLSPGERATLSCRASKGVSTSGYSYLHWYQQKPGQAPRLLIYLASYLESGVPARFSGSGSGTDFT
LTISSLEPEDFAVYYCQHSRDLPLTFGGGTKVEIK
Blinatumomab light chain CDRs
CDR-L1: RASSSVSYMN (SEQ ID NO: 79)
CDR-L2: DTSKVAS (SEQ ID NO: 80)
CDR-L3: QQWSSNPLT (SEQ ID NO: 81)
Pembrolizumab heavy chain CDRs
CDR-H1: GYTFTNYYMY (SEQ ID NO: 82)
CDR-H2: NPSNGG (SEQ ID NO: 83)
CDR-H3: RDYRFDMGFDY (SEQ ID NO: 84)
Pembrolizumab light chain CDRs
CDR-L1: RASKGVSTSGYSYLH (SEQ ID NO: 85)
CDR-L2: LASYLES (SEQ ID NO: 86)
CDR-L3: QHSRDLPLT (SEQ ID NO: 87)
SEQ ID NO: 19-Light Chain (DID-310)
Urelumab VL_human Kappa LC constant_(GGGGS)3_Pembrolizumab
VH_(GGGGS)4_Pembrolizumab VL(“(GGGGS)3” and “(GGGGS)4” disclosed as
SEQ ID NOS 23 and 24, respectively)
EIVLTQSPATLSLSPGERATLSCRASQSVSSYLAWYQQKPGQAPRLLIYDASNRATGIPARFSGSGSGTDFTLT
ISSLEPEDFAVYYCQQRSNWPPALTFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGECGGGGSG
GGGSGGGGSQVQLVQSGVEVKKPGASVKVSCKASGYTFTNYYMYWVRQAPGQGLEWMGGINPSNGGTNFNEKFK
NRVTLITDSSTITAYMELKSLQFDDTAVYYCARRDYRFDMGFDYWGQGTTVTVSSGGGGSGGGGSGGGGSGGGG
SEIVLTQSPATLSLSPGERATLSCRASKGVSTSGYSYLHWYQQKPGQAPRLLIYLASYLESGVPARFSGSGSGT
DFTLTISSLEPEDFAVYYCQHSRDLPLTFGGGTKVEIK
Urelumab light chain CDRs
CDR-L1: RASQSVSSYLA (SEQ ID NO: 88)
CDR-L2: DASNRAT (SEQ ID NO: 89)
CDR-L3: QQRSNWPPALT (SEQ ID NO: 90)
Pembrolizumab heavy chain CDRs
CDR-H1: GYTFTNYYMY (SEQ ID NO: 91)
CDR-H2: NPSNGG (SEQ ID NO: 92)
CDR-H3: RDYRFDMGFDY (SEQ ID NO: 93)
Pembrolizumab light chain CDRs
CDR-L1: RASKGVSTSGYSYLH (SEQ ID NO: 94)
CDR-L2: LASYLES (SEQ ID NO: 95)
CDR-L3: QHSRDLPLT (SEQ ID NO: 96)

1. A multispecific immunomodulatory antigen-binding construct (MIAC) polypeptide, comprising:
a. an antigen-binding module 1 (ABM1) that binds specifically to a HER2 antigen expressed by a cancer cell;
b. an antigen-binding module 2 (ABM2) that binds specifically to an activating receptor expressed by an effector immune cell, wherein binding of ABM2 to the activating receptor agonizes the activating receptor, and wherein the activating receptor is CD3 or CD137; and
c. optionally, an antigen-binding module 3 (ABM3) that binds specifically to an inhibitory receptor expressed by the effector immune cell, wherein the binding of ABM3 to the inhibitory receptor antagonizes the inhibitory receptor,
wherein ABM1, ABM2, and ABM3 are operably linked to each other, and
wherein each antigen binding module is capable of binding its respective antigen or receptor at the same time as each of the other antigen binding modules is bound to its respective antigen or receptor.

2. The MIAC of claim 1, wherein the MIAC further comprises Fc, wherein ABM1 is an scFv fragment, ABM2 is a Fab fragment, ABM2 is linked to the N terminus of Fc, and ABM1 is linked to the C terminus of Fc.

3. The MIAC of claim 1, wherein the MIAC further comprises Fc, wherein ABM1 is an scFv fragment, ABM2 is a Fab fragment, ABM3 is an scFv fragment, ABM2 is linked to the N terminus of Fc, ABM1 is linked to the C terminus of Fc, and ABM3 is linked to the C terminus of ABM2.

4. A multispecific immunomodulatory antigen-binding construct (MIAC) polypeptide, comprising:
a. an antigen-binding module 1 (ABM1) that binds specifically to a HER2 antigen expressed by a cancer cell;
b. optionally, an antigen-binding module 2 (ABM2) that binds specifically to an activating receptor expressed by an effector immune cell, wherein binding of ABM2 to the activating receptor agonizes the activating receptor; and
c. an antigen-binding module 3 (ABM3) that binds specifically to an inhibitory receptor expressed by the effector immune cell, wherein the binding of ABM3 to the inhibitory receptor antagonizes the inhibitory receptor, and wherein the inhibitory receptor is PD1,
wherein ABM1, ABM2, and ABM3 are operably linked to each other, and
wherein each antigen binding module is capable of binding its respective antigen or receptor at the same time as each of the other antigen binding modules is bound to its respective antigen or receptor.

5. The MIAC of claim 4, wherein the MIAC further comprises Fc, wherein ABM1 is an scFv fragment, ABM3 is a Fab fragment, ABM3 is linked to the N terminus of Fc, and ABM1 is linked to the C terminus of Fc.

6. The MIAC of claim 4, wherein the MIAC further comprises Fc, wherein ABM1 is an scFv fragment, ABM2 is a Fab fragment, ABM3 is an scFv fragment, ABM2 is linked to the N terminus of Fc, ABM1 is linked to the C terminus of Fc, and ABM3 is linked to the C terminus of ABM2.

7. The MIAC of claim 1 or 4, wherein the MIAC further comprises Fc, wherein ABM1 is an scFv fragment, ABM2 is a Fab fragment, and ABM3 is an scFv fragment, wherein ABM2 is linked to Fc, ABM3 is linked to ABM2, and ABM1 is linked to Fc, wherein the MIAC induces a greater amount of at least one of IFN-γ, TNF-α, IL-2, and granzyme B secretion by an effector immune cell upon binding to at least one effector immune cell and at least one cancer cell relative to a control set of antibodies, wherein the control set of antibodies consists of separate monospecific antibodies present at equimolar concentrations that collectively bind specifically to the same targets as the MIAC, wherein the MIAC induces a greater level of effector immune cell proliferation upon binding to at least one effector immune cell and at least one cancer cell relative to the control set of antibodies, and wherein the MIAC induces a greater level of effector immune cell CD25 cell surface expression upon binding to at least one effector immune cell and at least one cancer cell relative to the control set of antibodies.

8. The MIAC of claim 1 or 4, wherein the MIAC consists of ABM1, ABM2, ABM3, and Fc linked together, wherein ABM1 is an scFv fragment, ABM2 is a Fab fragment, and ABM3 is an scFv fragment, wherein the C terminus of the heavy chain of ABM2 is linked to the N terminus of Fc, ABM1 is linked to the C terminus of Fc, and ABM3 is linked to the C terminus of the light chain of ABM2, wherein the MIAC induces a greater amount of at least one of IFN-γ, TNF-α, IL-2, and granzyme B secretion by an effector immune cell upon binding to at least one effector immune cell and at least one cancer cell relative to a control set of antibodies, wherein the control set of antibodies consists of separate monospecific antibodies present at equimolar concentrations that collectively bind specifically to the same targets as the MIAC, wherein the MIAC induces a greater level of effector immune cell proliferation upon binding to at least one effector immune cell and at least one cancer cell relative to the control set of antibodies, and wherein the MIAC induces a greater level of effector immune cell CD25 cell surface expression upon binding to at least one effector immune cell and at least one cancer cell relative to the control set of antibodies.

9. The MIAC of claim 1 or 4, wherein the MIAC consists of ABM1, ABM2, ABM3, and Fc linked together, wherein ABM1 is an scFv fragment, ABM2 is a Fab fragment, and ABM3 is an scFv fragment, wherein the C terminus of the heavy chain of ABM2 is linked to the N terminus of Fc, ABM1 is linked to the C terminus of Fc, and ABM3 is linked to the C terminus of the light chain of ABM2.

10. The MIAC of claim 1 or 4, wherein the MIAC further comprises a scaffold, optionally wherein the scaffold is Fc, optionally wherein the Fc is human Fc, optionally wherein the Fc is human IgG Fc, optionally wherein each of ABM1, ABM2, and ABM3 is linked to the scaffold directly or indirectly with or without a linker, optionally wherein the linker is a polypeptide linker.

11. The MIAC of claim 10, wherein the scaffold comprises Fc.

12. The MIAC of claim 11, wherein Fc is an IgG (IgG1, IgG2, IgG3, IgG4), an IgA (IgA1, IgA2), an IgD, an IgE, or an IgM, optionally wherein Fc is modified, optionally wherein the modification reduces glycosylation, optionally wherein the modification reduces ADCC, optionally wherein the modification is an N297 mutation in human IgG1 Fc, optionally wherein the N297 mutation is a N297A mutation.

13. The MIAC of claim 11, wherein Fc is human IgG1 Fc.

14. The MIAC of claim 11, wherein ABM1 and ABM2 are linked to a position distinct from the C terminus of Fc; and ABM3 is linked to the C terminus of Fc.

15. The MIAC of claim 11, wherein ABM1 and ABM3 are linked to a position distinct from the C terminus of Fc; and ABM2 is linked to the C terminus of Fc.

16. The MIAC of claim 11, wherein ABM3 is linked to the C terminus of Fc.

17. The MIAC of claim 11, wherein ABM2 is linked to the C terminus of Fc.

18. The MIAC of claim 11, wherein ABM1 is linked to the N terminus of Fc.

19. The MIAC of claim 11, wherein ABM1 is a Fab fragment linked to the N terminus of Fc.

20. The MIAC of claim 11, wherein the ABMs and Fc are linked in a format that does not substantially interfere with ADCC directed against the cancer cell.

21. The MIAC of claim 11, wherein ABM3 and ABM2 are linked to a position distinct from the C terminus of Fc; and ABM1 is linked to the C terminus of Fc.

22. The MIAC of claim 11, wherein ABM3 is linked to the N terminus of Fc.

23. The MIAC of claim 11, wherein ABM2 is linked to the N terminus of Fc.

24. The MIAC of claim 11, wherein ABM1 is linked to the C terminus of Fc.

25. The MIAC of claim 11, wherein the ABMs and Fc are linked in a format that substantially interferes with ADCC directed against the cancer cell.

26. The MIAC of any above claim, wherein each of ABM1, ABM2, and ABM3 is an antibody or an antigen-binding fragment thereof.

27. The MIAC of claim 26, wherein the antibody or antigen-binding fragment thereof is an IgG (IgG1, IgG2, IgG3, IgG4), an IgA (IgA1, IgA2), an IgD, an IgE, an IgM, a DVD-Ig, and/or a heavy chain antibody.

28. The MIAC of claim 26, wherein the antibody or antigen-binding fragment thereof is an Fv fragment, a Fab fragment, a F(ab′)₂ fragment, a Fab′ fragment, an scFv fragment, an scFv-Fc fragment, and/or a single-domain antibody or antigen binding fragment thereof.

29. The MIAC of claim 26, wherein the antibody or antigen-binding fragment thereof is monoclonal, human, humanized, and/or chimeric.

30. The MIAC of any of the preceding claims, wherein at least one of ABM1, ABM2, and ABM3 further comprises an alternative scaffold, or wherein the MIAC further comprises an alternative scaffold.

31. The MIAC of any of the preceding claims, wherein the effector immune cell is a T cell or a natural killer (NK) cell, optionally wherein the T cell is a CD4+ helper T cell or a CD8+ cytotoxic T cell.

32. The MIAC of any of the preceding claims, wherein the cancer cell is a cell from HER2+ cancer, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

33. The MIAC of any of the preceding claims, wherein ABM2 comprises four immunoglobulin variable domains.

34. The MIAC of claim 33, wherein ABM1 comprises two immunoglobulin variable domains.

35. The MIAC of claim 34, wherein ABM3 comprises two immunoglobulin variable domains.

36. The MIAC of claim 35, wherein ABM2 is a Fab fragment, ABM1 is an scFv fragment, and ABM3 is an scFv fragment.

37. The MIAC of any above claim, wherein the MIAC further comprises Fc, and wherein ABM2 is linked to Fc, ABM3 is linked to ABM2, and ABM1 is linked to Fc.

38. The MIAC of claim 37, wherein the C terminus of the heavy chain of ABM2 is linked to the N terminus of Fc, ABM1 is linked to the C terminus of Fc, and ABM3 is linked to the C terminus of the light chain of ABM2.

39. The MIAC of any above claim, wherein each linkage is direct or via a linker, optionally wherein the linker is a polypeptide linker, optionally wherein the polypeptide linker is a gly-ser linker or an immunoglobulin hinge region or portion thereof, optionally wherein the linker is a (G₄S)₃ linker.

40. The MIAC of any above claim, wherein the MIAC is a dimer, optionally wherein the dimer is a homodimer.

41. The MIAC of any of the preceding claims, further comprising an antigen-binding module 4 (ABM4) that binds specifically to a further molecule expressed by the effector immune cell.

42. The MIAC of claim 41, wherein the further molecule expressed by the effector immune cell is selected from CD16 (CD16a, CD16b), CD32a, CD64, and CD89.

43. The MIAC of claim 41, wherein ABM4 is an Fc.

44. The MIAC of any of claims 1 to 43, wherein ABM2 is anti-CD137.

45. The MIAC of any of claims 1 to 43, wherein ABM2 is anti-CD3.

46. The MIAC of any of claims 1 to 43, wherein ABM3 is anti-PD1.

47. The MIAC of any of claims 1 to 43, wherein ABM1 is anti-HER2 and ABM2 is anti-CD3.

48. The MIAC of any of claims 1 to 43, wherein ABM1 is anti-HER2 and ABM2 is anti-CD137.

49. The MIAC of any of claims 1 to 43, wherein ABM1 is anti-HER2 and ABM3 is anti-PD1.

50. The MIAC of any of claims 1 to 43, wherein ABM1 is anti-HER2, ABM2 is anti-CD3, and ABM3 is anti-PD-1.

51. The MIAC of any of claims 1 to 43, wherein ABM1 is anti-HER2, ABM2 is anti-CD137, and ABM3 is anti-PD-1.

52. The MIAC of any of the preceding claims, wherein at least two of ABM1, ABM2, and ABM3 are covalently associated with each other.

53. The MIAC of claim 52, wherein the covalent association is in the form of a fusion protein.

54. The MIAC of any of the preceding claims, wherein at least two of ABM1, ABM2, and ABM3 are non-covalently associated with each other.

55. The MIAC of any preceding claim, wherein the MIAC induces a greater amount of at least one of IFN-γ, TNF-α, IL-2, and granzyme B secretion by an effector immune cell upon binding to at least one effector immune cell and at least one cancer cell relative to a control set of antibodies, wherein the control set of antibodies consists of separate monospecific antibodies present at equimolar concentrations that collectively bind specifically to the same targets as the MIAC.

56. The MIAC of claim 55, wherein the amount of IFN-γ, TNF-α, IL-2, and/or granzyme B secretion induced by the MIAC is about 2, 3, 4, 5, 6, 7, or 8-fold greater than that induced by the control set of antibodies.

57. The MIAC of any preceding claim, wherein the MIAC induces a greater level of effector immune cell proliferation upon binding to at least one effector immune cell and at least one cancer cell relative to a control set of antibodies, wherein the control set of antibodies consists of separate monospecific antibodies present at equimolar concentrations that collectively bind specifically to the same targets as the MIAC.

58. The MIAC of claim 57, wherein the level of proliferation induced by the MIAC is about 2, 3, 4, 5, 6, 7, or 8-fold greater than that induced by the control set of antibodies.

59. The MIAC of any preceding claim, wherein the MIAC induces a greater level of effector immune cell CD25 cell surface expression upon binding to at least one effector immune cell and at least one cancer cell relative to a control set of antibodies, wherein the control set of antibodies consists of separate monospecific antibodies present at equimolar concentrations that collectively bind specifically to the same targets as the MIAC.

60. The MIAC of claim 59, wherein the CD25 expression induced by the MIAC is about 2, 3, 4, 5, 6, 7, or 8-fold greater than that induced by the control set of antibodies.

61. The MIAC of any preceding claim, wherein the MIAC induces a greater level of cancer cell death upon binding to at least one effector immune cell and at least one cancer cell relative to a control set of antibodies, wherein the control set of antibodies consists of separate monospecific antibodies present at equimolar concentrations that collectively bind specifically to the same targets as the MIAC.

62. The MIAC of any preceding claim, wherein each of ABM binds its respective antigen or receptor at the same time as each of the other antigen binding modules is bound to its respective antigen or receptor, and optionally wherein the affinity of each binding module to its respective antigen or receptor is about 0.3 nM to about 1.7 nM, 0.37 to 1.66 nM, 0.3, 0.4, 0.5, 0.6, 0.7, 0.8, 0.9, 1.0, 1.1, 1.2, 1.3, 1.4, 1.5, 1.6, or 1.7 nM when each of ABM is simultaneously bound to its respective antigen or receptor.

63. A conjugate comprising the MIAC of any preceding claim and an agent.

64. The conjugate of claim 63, wherein the agent is selected from a therapeutic agent, a diagnostic agent, a masking moiety, a cleavable moiety, and combinations thereof.

65. The conjugate of claim 63, wherein the agent is attached to the MIAC with a linker.

66. A pharmaceutical composition comprising the MIAC or conjugate of any preceding claim and an excipient.

67. A method of treating a subject with cancer comprising administering an effective amount of the MIAC or conjugate of any preceding claim or the pharmaceutical composition of claim 66 to the subject.

68. A method of inhibiting or reducing cancer growth comprising contacting the cancer with an effective amount of the MIAC or conjugate of any preceding claim or the pharmaceutical composition of claim 66 to the subject.

69. The method of claim 67 or claim 68, wherein the MIAC binds a cancer cell and an effector cell.

70. The method of claim 69, wherein the MIAC binds two or more effector cells.

71. The method of any of claims 67-69, wherein the MIAC agonizes an activating receptor on the effector cell and antagonizes an inhibitory receptor on the effector cell.

72. The method of any of claims 67-71, wherein the MIAC activates the effector cell.

73. The method of any of claims 67-72, wherein the activated effector cell exhibits a phenotype selected from cytotoxicity toward cancer cells, proliferation, secretion of IL-2, secretion of interferon gamma, upregulation of LAMP-1, downregulation of CD16, upregulation of CD69, and upregulation of KLRG1.

74. The method of claim 73, wherein the proliferation induced by the MIAC is greater than proliferation induced by a MIAC without ABM3.

75. The method of any of claims 67-74, wherein the cancer is selected from HER2+ cancer, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

76. The method of any of claims 67-75, further comprising administering at least one further agent to the subject.

77. A composition comprising at least one polynucleotide or a set of polynucleotides encoding the MIAC of any of claims 1-62.

78. A cell comprising the composition of claim 77.

79. A method of making a MIAC, comprising expressing the MIAC in the cell of claim 78.

80. A method of making a MIAC, comprising expressing the ABMs of a MIAC of any of claims 1-62, and assembling the ABMs to form a MIAC.

81. A vector or set of vectors comprising at least one polynucleotide or a set of polynucleotides encoding the MIAC of any of claims 1-62.

82. A kit comprising the MIAC of any of claims 1-62 and instructions for use.


SEQUENCE LISTING (97 sequences)

SEQ ID NO: 1 - protein, 727 aa, Artificial Sequence
  Description of Artificial Sequence: Synthetic polypeptide
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSGGGGSGGGGSGGGGSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIK

SEQ ID NO: 2 - protein, 728 aa, Artificial Sequence
  Description of Artificial Sequence: Synthetic polypeptide
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 3 - protein, 218 aa, Artificial Sequence
  Description of Artificial Sequence: Synthetic polypeptide
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - skipped

SEQ ID NO: 5 - protein, 859 aa, Artificial Sequence
  Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAGGGGSGGGGSGGGGSGGGGSGGGGSGGGGSGGGGSDIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 6 - protein, 337 aa, Artificial Sequence
  Description of Artificial Sequence: Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 450 aa, Artificial Sequence
  Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 214 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 449 aa, Artificial Sequence
  Description of Artificial Sequence: Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, Artificial Sequence
  Description of Artificial Sequence: Synthetic polypeptide
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 451 aa, Artificial Sequence
  Description of Artificial Sequence: Synthetic polypeptide
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 216 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 450 aa, Artificial Sequence
  Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 218 aa, Artificial Sequence
  Description of Artificial Sequence: Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 711 aa, Artificial Sequence
  Description of Artificial Sequence: Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 16 - protein, 713 aa, Artificial Sequence
  Description of Artificial Sequence: Synthetic polypeptide
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 17 - protein, 712 aa, Artificial Sequence
  Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 18 - protein, 479 aa, Artificial Sequence
  Description of Artificial Sequence: Synthetic polypeptide
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 19 - protein, 482 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 20 - protein, 5 aa, Artificial Sequence
  Description of Artificial Sequence: Synthetic peptide
GGGGS

SEQ ID NO: 21 - protein, 26 aa, Homo sapiens
NSTYRVVSVLTVLHQDWLNGKEYKCK

SEQ ID NO: 22 - protein, 25 aa, Homo sapiens
FQNALLVRYTKKVPQVSTPTLVEVS

SEQ ID NO: 23 - protein, 15 aa, Artificial Sequence
  Description of Artificial Sequence: Synthetic peptide
GGGGSGGGGSGGGGS

SEQ ID NO: 24 - protein, 20 aa, Artificial Sequence
  Description of Artificial Sequence: Synthetic peptide
GGGGSGGGGSGGGGSGGGGS

SEQ ID NO: 25 - protein, 5 aa, Artificial Sequence
  Description of Artificial Sequence: Synthetic peptide
GGGGS

SEQ ID NO: 26 - protein, 10 aa, Artificial Sequence
  Description of Artificial Sequence: Synthetic peptide
GGGGSGGGGS

SEQ ID NO: 27 - protein, 25 aa, Artificial Sequence
  Description of Artificial Sequence: Synthetic peptide
GGGGSGGGGSGGGGSGGGGSGGGGS

SEQ ID NO: 28 - protein, 7 aa, Artificial Sequence
  Description of Artificial Sequence: Synthetic peptide
GFNIKDT

SEQ ID NO: 29 - protein, 6 aa, Artificial Sequence
  Description of Artificial Sequence: Synthetic peptide
YPTNGY

SEQ ID NO: 30 - protein, 11 aa, Artificial Sequence
  Description of Artificial Sequence: Synthetic peptide
WGGDGFYAMDY

SEQ ID NO: 31 - protein, 11 aa, Artificial Sequence
  Description of Artificial Sequence: Synthetic peptide
RASQDVNTAVA

SEQ ID NO: 32 - protein, 7 aa, Artificial Sequence
  Description of Artificial Sequence: Synthetic peptide
SASFLYS

SEQ ID NO: 33 - protein, 9 aa, Artificial Sequence
  Description of Artificial Sequence: Synthetic peptide
QQHYTTPPT

SEQ ID NO: 34 - protein, 10 aa, Artificial Sequence
  Description of Artificial Sequence: Synthetic peptide
GYTFTRYTMH

SEQ ID NO: 35 - protein, 9 aa, Artificial Sequence
  Description of Artificial Sequence: Synthetic peptide
YINPSRGYT

SEQ ID NO: 36 - protein, 10 aa, Artificial Sequence
  Description of Artificial Sequence: Synthetic peptide
YYDDHYCLDY

SEQ ID NO: 37 - protein, 10 aa, Artificial Sequence
  Description of Artificial Sequence: Synthetic peptide
RASSSVSYMN

SEQ ID NO: 38 - protein, 7 aa, Artificial Sequence
  Description of Artificial Sequence: Synthetic peptide
DTSKVAS

SEQ ID NO: 39 - protein, 9 aa, Artificial Sequence
  Description of Artificial Sequence: Synthetic peptide
QQWSSNPLT

SEQ ID NO: 40 - protein, 8 aa, Artificial Sequence
  Description of Artificial Sequence: Synthetic peptide
GGSFSGYY

SEQ ID NO: 41 - protein, 5 aa, Artificial Sequence
  Description of Artificial Sequence: Synthetic peptide
NHGGY

SEQ ID NO: 42 - protein, 13 aa, Artificial Sequence
  Description of Artificial Sequence: Synthetic peptide
DYGPGNYDWYFDL

SEQ ID NO: 43 - protein, 11 aa, Artificial Sequence
  Description of Artificial Sequence: Synthetic peptide
RASQSVSSYLA

SEQ ID NO: 44 - protein, 7 aa, Artificial Sequence
  Description of Artificial Sequence: Synthetic peptide
DASNRAT

SEQ ID NO: 45 - protein, 11 aa, Artificial Sequence
  Description of Artificial Sequence: Synthetic peptide
QQRSNWPPALT

SEQ ID NO: 46 - protein, 10 aa, Artificial Sequence
  Description of Artificial Sequence: Synthetic peptide
GYTFTNYYMY

SEQ ID NO: 47 - protein, 6 aa, Artificial Sequence
  Description of Artificial Sequence: Synthetic peptide
NPSNGG

SEQ ID NO: 48 - protein, 11 aa, Artificial Sequence
  Description of Artificial Sequence: Synthetic peptide
RDYRFDMGFDY

SEQ ID NO: 49 - protein, 15 aa, Artificial Sequence
  Description of Artificial Sequence: Synthetic peptide
RASKGVSTSGYSYLH

SEQ ID NO: 50 - protein, 7 aa, Artificial Sequence
  Description of Artificial Sequence: Synthetic peptide
LASYLES

SEQ ID NO: 51 - protein, 9 aa, Artificial Sequence
  Description of Artificial Sequence: Synthetic peptide
QHSRDLPLT

SEQ ID NO: 52 - protein, 10 aa, Artificial Sequence
  Description of Artificial Sequence: Synthetic peptide
GYTFTRYTMH

SEQ ID NO: 53 - protein, 9 aa, Artificial Sequence
  Description of Artificial Sequence: Synthetic peptide
YINPSRGYT

SEQ ID NO: 54 - protein, 10 aa, Artificial Sequence
  Description of Artificial Sequence: Synthetic peptide
YYDDHYCLDY

SEQ ID NO: 55 - protein, 7 aa, Artificial Sequence
  Description of Artificial Sequence: Synthetic peptide
GFNIKDT

SEQ ID NO: 56 - protein, 6 aa, Artificial Sequence
  Description of Artificial Sequence: Synthetic peptide
YPTNGY

SEQ ID NO: 57 - protein, 11 aa, Artificial Sequence
  Description of Artificial Sequence: Synthetic peptide
WGGDGFYAMDY

SEQ ID NO: 58 - protein, 11 aa, Artificial Sequence
  Description of Artificial Sequence: Synthetic peptide
RASQDVNTAVA

SEQ ID NO: 59 - protein, 7 aa, Artificial Sequence
  Description of Artificial Sequence: Synthetic peptide
SASFLYS

SEQ ID NO: 60 - protein, 9 aa, Artificial Sequence
  Description of Artificial Sequence: Synthetic peptide
QQHYTTPPT

SEQ ID NO: 61 - protein, 8 aa, Artificial Sequence
  Description of Artificial Sequence: Synthetic peptide
GGSFSGYY

SEQ ID NO: 62 - protein, 5 aa, Artificial Sequence
  Description of Artificial Sequence: Synthetic peptide
NHGGY

SEQ ID NO: 63 - protein, 13 aa, Artificial Sequence
  Description of Artificial Sequence: Synthetic peptide
DYGPGNYDWYFDL

SEQ ID NO: 64 - protein, 7 aa, Artificial Sequence
  Description of Artificial Sequence: Synthetic peptide
GFNIKDT

SEQ ID NO: 65 - protein, 6 aa, Artificial Sequence
  Description of Artificial Sequence: Synthetic peptide
YPTNGY

SEQ ID NO: 66 - protein, 11 aa, Artificial Sequence
  Description of Artificial Sequence: Synthetic peptide
WGGDGFYAMDY

SEQ ID NO: 67 - protein, 11 aa, Artificial Sequence
  Description of Artificial Sequence: Synthetic peptide
RASQDVNTAVA

SEQ ID NO: 68 - protein, 7 aa, Artificial Sequence
  Description of Artificial Sequence: Synthetic peptide
SASFLYS

SEQ ID NO: 69 - protein, 9 aa, Artificial Sequence
  Description of Artificial Sequence: Synthetic peptide
QQHYTTPPT

SEQ ID NO: 70 - protein, 10 aa, Artificial Sequence
  Description of Artificial Sequence: Synthetic peptide
GYTFTNYYMY

SEQ ID NO: 71 - protein, 6 aa, Artificial Sequence
  Description of Artificial Sequence: Synthetic peptide
NPSNGG

SEQ ID NO: 72 - protein, 11 aa, Artificial Sequence
  Description of Artificial Sequence: Synthetic peptide
RDYRFDMGFDY

SEQ ID NO: 73 - protein, 7 aa, Artificial Sequence
  Description of Artificial Sequence: Synthetic peptide
GFNIKDT

SEQ ID NO: 74 - protein, 6 aa, Artificial Sequence
  Description of Artificial Sequence: Synthetic peptide
YPTNGY

SEQ ID NO: 75 - protein, 11 aa, Artificial Sequence
  Description of Artificial Sequence: Synthetic peptide
WGGDGFYAMDY

SEQ ID NO: 76 - protein, 11 aa, Artificial Sequence
  Description of Artificial Sequence: Synthetic peptide
RASQDVNTAVA

SEQ ID NO: 77 - protein, 7 aa, Artificial Sequence
  Description of Artificial Sequence: Synthetic peptide
SASFLYS

SEQ ID NO: 78 - protein, 9 aa, Artificial Sequence
  Description of Artificial Sequence: Synthetic peptide
QQHYTTPPT

SEQ ID NO: 79 - protein, 10 aa, Artificial Sequence
  Description of Artificial Sequence: Synthetic peptide
RASSSVSYMN

SEQ ID NO: 80 - protein, 7 aa, Artificial Sequence
  Description of Artificial Sequence: Synthetic peptide
DTSKVAS

SEQ ID NO: 81 - protein, 9 aa, Artificial Sequence
  Description of Artificial Sequence: Synthetic peptide
QQWSSNPLT

SEQ ID NO: 82 - protein, 10 aa, Artificial Sequence
  Description of Artificial Sequence: Synthetic peptide
GYTFTNYYMY

SEQ ID NO: 83 - protein, 6 aa, Artificial Sequence
  Description of Artificial Sequence: Synthetic peptide
NPSNGG

SEQ ID NO: 84 - protein, 11 aa, Artificial Sequence
  Description of Artificial Sequence: Synthetic peptide
RDYRFDMGFDY

SEQ ID NO: 85 - protein, 15 aa, Artificial Sequence
  Description of Artificial Sequence: Synthetic peptide
RASKGVSTSGYSYLH

SEQ ID NO: 86 - protein, 7 aa, Artificial Sequence
  Description of Artificial Sequence: Synthetic peptide
LASYLES

SEQ ID NO: 87 - protein, 9 aa, Artificial Sequence
  Description of Artificial Sequence: Synthetic peptide
QHSRDLPLT

SEQ ID NO: 88 - protein, 11 aa, Artificial Sequence
  Description of Artificial Sequence: Synthetic peptide
RASQSVSSYLA

SEQ ID NO: 89 - protein, 7 aa, Artificial Sequence
  Description of Artificial Sequence: Synthetic peptide
DASNRAT

SEQ ID NO: 90 - protein, 11 aa, Artificial Sequence
  Description of Artificial Sequence: Synthetic peptide
QQRSNWPPALT

SEQ ID NO: 91 - protein, 10 aa, Artificial Sequence
  Description of Artificial Sequence: Synthetic peptide
GYTFTNYYMY

SEQ ID NO: 92 - protein, 6 aa, Artificial Sequence
  Description of Artificial Sequence: Synthetic peptide
NPSNGG

SEQ ID NO: 93 - protein, 11 aa, Artificial Sequence
  Description of Artificial Sequence: Synthetic peptide
RDYRFDMGFDY

SEQ ID NO: 94 - protein, 15 aa, Artificial Sequence
  Description of Artificial Sequence: Synthetic peptide
RASKGVSTSGYSYLH

SEQ ID NO: 95 - protein, 7 aa, Artificial Sequence
  Description of Artificial Sequence: Synthetic peptide
LASYLES

SEQ ID NO: 96 - protein, 9 aa, Artificial Sequence
  Description of Artificial Sequence: Synthetic peptide
QHSRDLPLT

SEQ ID NO: 97 - protein, 35 aa, Artificial Sequence
  Description of Artificial Sequence: Synthetic polypeptide
GGGGSGGGGSGGGGSGGGGSGGGGSGGGGSGGGGS